(19) United States
(12) Patent Application Publication
(10) Pub. No.: US 2018/0064679 A1
(43) Pub. Date: Mar. 8, 2018

(54) METHOD FOR TREATING CANCER BASED ON LEVEL OF GLUCOCORTICOID RECEPTOR

(71) Applicant: Abraxis BioScience, LLC, Los Angeles, CA (US)

(72) Inventors: Daniel W. PIERCE, Belmont, CA (US); Marianna ZAVODOVSKAYA, Woodside, CA (US); Jinhong FAN, San Mateo, CA (US)

(73) Assignee: Abraxis BioScience, LLC, Los Angeles, CA (US)

(21) Appl. No.: 15/555,310

(22) PCT Filed: Mar. 4, 2016
(86) PCT No.: PCT/US2016/021077

§ 371 Date: Sep. 1, 2017

Related U.S. Application Data
(60) Provisional application No. 62/129,008, filed on Mar. 5, 2015.

(51) Int. Cl. A61K 31/337; A61K 9/51; A61K 31/567; A61K 31/573

(52) U.S. Cl. CPC A61K 31/337; A61K 31/573; A61K 31/567; A61K 9/5169

(57) ABSTRACT

The present invention provides methods and compositions for treating cancer with taxane-based therapy. The individuals may have a high level of glucocorticoid receptor (GR) and/or a high level of glucocorticoid (GC). The taxane may be combined with another agent that down-regulates GR.

### GROSS REFERENCE TO RELATED APPLICATIONS

This application claims priority benefit of U.S. Provisional Application No. 62/129,008, filed Mar. 5, 2015, the content of which is incorporated herein by reference in its entirety.

### TECHNICAL HELD

The present invention relates to methods and compositions for treating cancer comprising administering compositions comprising taxane (e.g., paclitaxel).

### BACKGROUND

Albumin-based nanoparticle compositions have been developed as a drug delivery system for delivering substantially water insoluble drugs such as taxanes. See, for example, U.S. Pat. Nos. 5,916,596; 6,506,405; 6,749,868, and 6,537,579, 7,820,788, and 7,923,536. ABRAXANE®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as pancreatic cancer and melanoma.

Albumin-based paclitaxel nanoparticle compositions (e.g., ABRAXANE®) in combination with gemcitabine was found to be well tolerated in advanced pancreatic cancer in a Phase I/II study and showed evidence of antitumor activity. See, for example, US Patent App.; No. 2006/0263434; Maitra et al., Mol. Cancer Ther. 8(12 Suppl): C246 (2009); Loehr et al., J. of Clinical Oncology 27 (15S) (May 20 Supplement): 200, Abstract No. 4526 (2009); Von Hoff et al., J. of Clinical Oncology 27(15S) (May 20 Supplement), Abstract No. 4525 (2009); and Kim et al., Proc. Amer. Assoc. Cancer Res., 46, Abstract No. 1440 (2005).

Endogenous glucocorticoids (GCs) are essential steroid hormones that participate in the maintenance of several key developmental and physiological processes in animals. Some glucocorticoids (such as cortisol) function by binding to the glucocorticoid receptor (GR), a ubiquitously expressed nuclear hormone receptor that regulates cellular metabolism, limits inflammatory responses, and promotes cell survival in a cell-type dependent manner. Glucocorticoid receptor agonists, including synthetic glucocorticoids, such as dexamethasone (DEX), are widely prescribed as a premedication to reduce edema, treat nausea and emesis, and stimulate appetite in patients receiving chemotherapy. It is not known whether effects on nausea and appetite are mediated by GR. Among advanced and end-stage cancer patients, many routinely take glucocorticoids to alleviate pain, fatigue and anorexia.

Glucocorticoids (such as DEX) are particularly important components in chemotherapy regimens involving taxanes (such as paclitaxel) that have poor water solubility. Solvent-based paclitaxel compositions (such as TAXOL®) are known to cause serious or fatal hypersensitivity reactions to the organic solvent in the formulations. As a result, patients are required to receive premedication with glucocorticoids (such as dexamethasone) prior to chemotherapy with solvent-based paclitaxel compositions. On the other hand, albumin-based paclitaxel nanoparticle compositions (such as Nab-paclitaxel, including ABRAXANE®) are essentially free of any organic solvent in the formulations, and thus do not require glucocorticoid premedication.

A developing body of evidence suggests that administration of glucocorticoids in conjunction with chemotherapy agents may reduce anti-tumor efficacy of the chemotherapy agents. The inventors of this application discovered that tumors with high glucocorticoid receptor expression or activity may be especially susceptible to the counterproductive effect of glucocorticoid administration in taxane (such as paclitaxel) chemotherapy regimens. Accordingly, the present invention provides methods and compositions for treating cancer in an individual by administering an albumin-based taxane composition (such as Nab-paclitaxel), and optionally a second agent that inhibits glucocorticoid receptor based upon the level of glucocorticoid receptor or glucocorticoid in the individual.

The disclosures of all publications, patents, patent applications and published patent applications referred to herein are hereby incorporated herein by reference in their entirety.

### BRIEF SUMMARY OF THE INVENTION

The present application provides methods and compositions for treating an individual having a cancer with taxane-based therapy, wherein the individual may have a high level of glucocorticoid receptor (GR) and/or a high level of glucocorticoid (GC). The taxane-based therapy may be combined with another agent that down-regulates GR.

One aspect of the present invention provides a method of treating an individual having a cancer, wherein the individual is characterized by a high level of glucocorticoid receptor (GR), comprising administering to the individual an effective amount of a composition comprising a taxane.

One aspect of the present invention provides a method of treating an individual having a cancer, wherein the individual is characterized by a high level of glucocorticoid (GC), comprising administering to the individual an effective amount of a composition comprising a taxane. In some embodiments, the cancer is further characterized by a high level of GR.

One aspect of the present invention provides a method of treating an individual having a cancer, comprising administering to the individual: a) an effective amount of a composition comprising a taxane; and b) an effective amount of another agent that down-regulates GR. In some embodiments, the individual is characterized by a high level of GR. In some embodiments, the individual is characterized by a high level of GC.

In some embodiments according to any one of the methods described above, a high level of GR is used as a basis for selecting the individual for treatment. In some embodiments, the method further comprises determining the level of GR in the individual.

In some embodiments according to any one of the methods described above, a high level of GC is used as a basis for selecting the individual for treatment. In some embodiments, the method further comprises determining the level of GC in the individual.

In some embodiments according to any one of the methods described above, the individual is characterized by a high level of GR expression.

In some embodiments according to any one of the methods described above, the individual is characterized by a high level of GR activity. In some embodiments, the high level of GR activity is determined by measuring the expression or activity of a GR responsive molecule. In some embodiments, the GR responsive molecule is selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG, SERPINE1, RGS2, KRT7, MME, JAK2, CEBPD, IL6, LIF, and TNFRSF11B.

In some embodiments according to any one of the methods described above, the individual is characterized by a high level of GC secretion.

In some embodiments according to any one of the methods described above, the individual is characterized, by high level of GC activity.

In some embodiments according to any one of the methods described above, the other agent is an inhibitor of GR expression.

In some embodiments according to any one of the combination therapy methods described above, the other agent is an inhibitor of GR activity. In some embodiments, the other agent is a GR antagonist. In some embodiments, the other agent is a modulator of a GR responsive molecule. In some embodiments, the GR responsive molecule is selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG,SERPINE1, RGS2, KRT7, MME, JAK2, CEBPD, IL6, LIF, and TNFRSF11B.

In some embodiments according to any one of the methods described above, the cancer is selected from the group consisting of breast cancer, lung cancer, and pancreatic cancer. In some embodiments, the cancer is pancreatic cancer.

In some embodiments according to any one of the methods described above, the cancer is advanced cancer.

In some embodiments according to any one of the combination therapy methods described above, the composition comprising the taxane and the other agent are administered simultaneously.

In some embodiments according to any one of the combination therapy methods described above, the composition comprising the taxane and the other agent are administered sequentially.

In some embodiments according to any one of the methods described above, the composition comprising the taxane is administered intravenously.

In some embodiments according to any one of the methods described above, the taxane is paclitaxel.

In some embodiments according to any one of the methods described above, the composition comprises nanoparticles comprising the taxane. In some embodiments, the composition comprises nanoparticles comprising the taxane and an albumin. In some embodiments, the nanoparticles in the composition comprise the taxane coated with the albumin. In some embodiments, the nanoparticles in the composition have an average diameter of no greater than about 200 nm. In some embodiments, the albumin is human albumin.

In some embodiments according to any one of the methods described above, the individual is human.

Also provided are compositions (such as pharmaceutical compositions), medicine, kits, and unit dosages useful for methods described herein.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE FIGURES

FIGS. 1A-1C show levels of apoptosis in MDA-MB-231 cells following treatment with DEX and/or PTX. FIG. 1A illustrates an array of MDA-MB-231 cell line samples measured for caspase-3/7 activation at time points following administration with at a specified paclitaxel (PTX) concentration. Each box represents measurements of a single sample over time. The PTX concentration decreases from left to right. FIG. 1B shows an array of MDA-MB-231 cell line samples measured for caspase-3/7 activation at time points following administration with 100 nM dexamethasone and a specified paclitaxel (PTX) concentration. Each box represents measurements of a single sample over time. The PTX concentration decrease from left to right. FIG. 1C shows a plot of caspase-3/7 activation (as reported by object count per image) versus time for MDA-MB-231 cell samples treated with either 333.33 nM PTX or 333.33 nM PTX and 100 nM DEX.

FIGS. 2A-2C show levels of apoptosis in H1755 cells following administration of DEX and/or PTX. FIG. 2A shows a plot of caspase-3/7 activation versus time for H1755 cell samples following administration of 0 nM DEX or 100 nM DEX. FIG. 2B shows a plot of caspase-3/7 activation versus time for H1755 cell samples following administration of 111 nM PTX and 0 nM DEX, 111 nM PTX and 100 nM DEX, or 0 nM PTX and 0 nM DEX. FIG. 2C shows a plot of caspase-3/7 activation versus concentration of DEX for H1755 cell samples following administration of a range of PTX concentrations.

FIGS. 3A-3D show representative images of apoptotic or apoptosing H1755 cell samples 40 hours after administration of DEX and/or PTX. Cells with detectable caspase-3/7 activation (i.e., apoptotic or apoptosing cells) are depicted in white. FIG. 3A shows a representative image of caspase-3/7 activation for a H1755 cell sample 40 hours after administration of 0 nM PTX and 0 nM DEX. FIG. 3B shows a representative image of caspase-3/7 activation for a H1755 cell sample 40 hours after administration of 0 nM PTX and 100 nM DEX. FIG. 3C shows a representative image of caspase-3/7 activation for a H1755 cell sample 40 hours after administration of 111 nM PTX and 0 nM DEX. FIG. 3D shows a representative image of caspase-3/7 activation for a H1755 cell sample 40 hours after administration of 111 nM PTX and 100 nM DEX.

FIGS. 4A-4D show the level of GR expression and DEX antagonism of PIX-induced apoptosis for NSCLC cell lines. FIG. 4A shows relative amounts of glucocorticoid receptor (GR) (based on GR/GAPDH measurements made using Western blot analysis) for NSCLC cell lines. FIG. 4B shows caspase-3/7 activation measurements of A549 cell samples following administration of 0 nM FIX and 0 nM DEX, 0 nM PTX and 100 nM DEX, 100 nM PTX and 100 nM DEX, or 100 nM PTX and 100 nM DEX. FIG. 4C shows caspase-3/7 activation measurements of H1755 cell samples following administration of 0 nM PTX and 0 nM DEX, 0 nM PTX and 100 nM DEX, 100 nM PTX and 100 nM DEX, or 100 nM PTX and 100 nM DEX. FIG. 4D shows caspase-3/7 activation measurements of H522 cell samples following administration of 0 nM PTX and 0 nM DEX, 0 nM PTX and 100 nM DEX, 100 nM PTX and 100 nM DEX, or 100 nM PTX and 100 nM DEX.

FIGS. 5A-5D show the level of GR expression and DEX antagonism of PTX-induced apoptosis for triple-negative breast cancer (TNBC) cell lines. FIG. 5A shows relative amounts of glucocorticoid receptor (GR) (based on GR/GAPDH measurements made using Western blot analysis) for TNBC cell lines. FIG. 5B shows caspase-3/7 activation measurements of MM231 cells following administration of 0 nM PTX and 0 nM DEX, 0 nM PTX and 100 nM DEX, 100 nM PTX and 100 nM DEX, or 100 nM PTX and 100 nM DEX. FIG. 5C shows caspase-3/7 activation measurements of CAL120 cells following administration of 0 nM PTX and 0 nM DEX, 0 nM PTX and 100 nM DEX, 100 nM PTX and 100 nM DEX, or 100 nM PTX and 100 nM DEX. FIG. 5D shows caspase-3/7 activation measurements of BT549 cells following administration of 0 nM PTX and 0 nM DEX, 0 nM PTX and 100 nM DEX, 100 nM PTX and 100 nM DEX, or 100 nM PTX and 100 nM DEX.

FIGS. 6A-6D show the level of GR expression and DEX antagonism of PTX-induced apoptosis for pancreatic ductal adenocarcinoma (PDAC) cell lines. FIG. 6A shows relative amounts of glucocorticoid receptor (GR) (based on GR/GAPDH measurements made using Western blot analysis) for PDAC cell lines. FIG. 6B shows caspase-3/7 activation measurements of HS766t cells following administration of 0 nM PTX and 0 nM DEX, 0 nM PTX and 100 nM DEX, 100 nM PTX and 100 nM DEX, or 100 nM PTX and 100 nM DEX. FIG. 6C shows caspase-3/7 activation measurements of Panc03.27 cells following administration of 0 nM PTX and 0 nM DEX, 0 nM PTX and 100 nM DEX, 100 nM PTX and 100 nM DEX, or 100 nM PTX and 100 nM DEX. FIG. 6D shows caspase-3/7 activation measurements of AsPC1 cells following administration of 0 nM PTX and 0 nM DEX, 0 nM PTX and 100 nM DEX, 100 nM PTX and 100 nM DEX, or 100 nM PTX and 100 nM DEX.

FIGS. 7A-7D show level of apoptosis inhibition (as mediated by DEX) and the level of GR expression for 20 cell lines. FIG. 7A shows inhibition of apoptosis (based on an apoptosis inhibition index) for NSCLC cell lines at 66 hours following administration of PTX and/or DEX. FIG. 7B shows inhibition of apoptosis (based on an apoptosis inhibition index) for TNBC cell lines at 66 hours following administration of PTX and/or DEX. FIG. 7C shows inhibition of apoptosis (based on an apoptosis inhibition index) for PDAC cell lines at 66 hours following administration of PTX and/or DEX. FIG. 7D shows inhibition of apoptosis (as measured by an apoptosis inhibition index) versus GR expression level for NSCLC, TNBC, and PDAC.

FIGS. 8A-8D show mRNA expression levels of genes in H1755 and H522 cells following DEX and/or PTX administration. FIG. 8A shows relative expression of MKP-1 mRNA in H1755 cells following administration of PTA and/or DEX at 1 hour, 4 hours, and 24 hours. FIG. 8B shows relative expression of MKP-1 mRNA in H522 cells following administration of PTX and/or DEX at 1 hour, 4 hours, and 24 hours. FIG. 8C shows relative expression of SGK1 mRNA in H1755 cells following administration of PTX and/or DEX at 1 hour, 4 hours, and 24 hours. FIG. 8D shows relative expression of SGK1 mRNA in H522 cells following administration of PTX and/or DEX at 1 hour, 4 hours, and 24 hours.

FIG. 9 shows alterations of protein expression levels in H1755 and H522 cell lines following treatment with DEX and/or PTX. Shown is the Western blot analysis of proteins and phosphorylated proteins in H1755 and H522 cell lines following PTX and/or DEX treatment.

FIGS. 10A-10D show alterations of protein expression levels in H1755 and H522 cell lines following treatment with DEX and/or PTX. FIG. 10A shows Western blot analysis of proteins and phosphorylated proteins in H1755 and H522 cell lines following treatment with PTX and/or DEX treatment. FIGS. 10B-10D show levels of MCL1 (FIG. 10B), phosphorylated BCL2 (FIG. 10C), and BCLXL (FIG. 10D) for H1755 and H522 cell lines.

FIGS. 11A-11B show alterations of MAP TAU protein expression levels in H1755 and H522 cell lines following treatment with DEX and/or PTX. FIG. 11A shows the Western blot analysis of MAP TAU in H1755 and H522 cell lines following treatment with PTX and/or DEX treatment. FIG. 11B shows levels of MAP TAU (normalized to control) in H1755 and H522 cells 24 hours after administration of DEX and/or PTX.

FIGS. 12A-12C show levels of apoptosis in H1755 cell line following GR knockdown. FIG. 12A shows caspase-3/7 activation measurements of H1755 cells following administration of 0 nM PTX and 0 nM DEX, 0 nM PTX and 100 nM DEX, 100 nM PTX and 100 nM DEX, or 100 nM PTX and 100 nM DEX. FIG. 12B shows caspase-3/7 activation measurements of H1755 cells (with shRNA knockdown of GR) following administration of 0 nM PTX and 0 nM DEX, 0 nM PTX and 100 nM DEX, 100 nM PTX and 100 nM DEX, or 100 nM PTX and 100 nM DEX. FIG. 12C shows inhibition of apoptosis (based on an apoptosis inhibition index) for H1755 cell samples with and without GR knockdown at 66 hours following administration of PTX and/or DEX.

FIG. 13 shows a plot of the relative expression profile of NR3C1 (the gene for GR) in solid tumor samples from patients. Data was sourced from The Cancer Genome Atlas (TCGA).

FIG. 14 shows a plot of the expression profile of GR in tumor cell lines. Data was sourced from the Cancer Cell Line Encyclopedia database.

FIG. 15 shows levels of apoptosis in H1755 cell samples following administration of PTX and/or DEX. Illustrated are plots of caspase-3/7 activation measurements of H1755 cells following administration of a range of DEX concentrations at a specified PTX concentration.

FIG. 16 shows determination of EC₅₀ for DEX-mediated rescue of PTX-induced apoptosis. Illustrated is a plot of caspase-3/7 activation versus DEX concentration.

FIG. 17 shows expression levels of MAPK pathway protein 4 hours after treatment with DEX and/or PTX. Shown is the Western blot analysis of proteins and phosphorylated proteins in H1755 and H522 cell lines following PTX and/or DEX treatment.

FIG. 18 shows expression levels of proteins involved in PTX and DEX response 24 and 48 hours after treatment. Shown is the Western blot analysis of proteins and phosphorylated proteins in H1755 and H522 cell lines following PTX and/or DEX treatment.

FIG. 19A shows expression levels of CALD1, CD274, CDH1, CEBPD, CXCL8, FN1, HRK, IL11, and IL18R1 in 8 cancer cell lines under DEX treatment (y-axis) or control conditions (x-axis).

FIG. 19B shows expression levels of IL1R1, IL6, JAK2, KRT7, LIF, LIFR, MCL1, MME, and MMP3 in 8 cancer cell lines under DEX treatment (y-axis) or control conditions (x-axis).

FIG. 19C shows expression levels of MP9, OCLN, RGS2, SERPINE1, SNAI2 (also known as SLUG), SOCS1, STEAP1, and TNFRSF11B in 8 cancer cell lines under DEX treatment (y-axis) or control conditions (x-axis).

### DETAILED DESCRIPTION OF THE INVENTION

The present invention provides methods of cancer treatment comprising administration of a composition comprising a taxane (such as compositions comprising nanoparticles comprising a taxane, for example compositions comprising nanoparticles comprising a taxane and an albumin). We report herein that glucocorticoid (such as dexamethasone), which is routinely used as a premedication in many taxane-based drug formulations, negatively impact the activity of taxane, for example by interrupting taxane-induced apoptosis. Taxane formulations that do not require premedication, such as nanoparticle compositions comprising albumin and taxane, would be particularly beneficial for treating cancer in individuals who have a high level of glucocorticoid receptor and/or a high level of glucocorticoid (such as endogenous glucocorticoid, for example cortisol). Further, combining a composition comprising taxane with another agent that down-regulates GR expression, reduces its activity, or blocks the activity of its downstream effectors would produce beneficial results in treating cancer, particularly in individuals having a high level of glucocorticoid receptor and/or a high level of glucocorticoid (such as endogenous glucocorticoid, for example cortisol).

The present application thus in one aspect provides a method of treating an individual having a cancer, wherein the individual is characterized by a high level of glucocorticoid receptor (GR) and/or a high level of glucocorticoid (GC, such as cortisol), comprising administering to the individual an effective amount of a composition comprising a taxane.

In another aspect, there is provided a method of treating an individual having a cancer, comprising administering to the individual: a) an effective amount of a composition comprising a taxane and b) an effective amount of another agent that down-regulates GR. The individual in some embodiments is characterized by a high level of glucocorticoid receptor (GR) and/or a high level of glucocorticoid (GC, such as cortisol).

Also provided are compositions (such as pharmaceutical compositions), medicine, kits, and unit dosages useful for the methods described herein.

### Definitions

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “individual” refers to a mammal and includes, but is not limited to, human, bovine, horse, feline, canine, rodent, or primate. In some embodiments, the individual is a human.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer. An individual having one or more of these risk factors has a higher probability of developing cancer than an individual without these risk factor(s).

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein, “delaying” the development of cancer means to defer, hinder, slow, retard, stabilize, arid/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

As used herein, by “combination therapy” is meant that a first agent be administered in conjunction with another agent. “In conjunction with” refers to administration of one treatment modality in addition to another treatment modality, such as administration of a taxane composition described herein in addition to administration of the other agent to the same individual. As such, “in conjunction with” refers to administration of one treatment modality before, during, or after delivery of the other treatment modality to the individual. Such combinations are considered to be part of a single treatment regime or regimen.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other,

As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

An “adverse event” or “AE” as used herein refers to any untoward medical occurrence in an individual receiving a marketed pharmaceutical product or in an individual who is participating on a clinical trial who is receiving an investigational or non-investigational pharmaceutical agent. The AE does not necessarily have a causal relationship with the individual's treatment. Therefore, an AE can be any unfavorable and unintended sign, symptom, or disease temporally associated with the use of a medicinal product, whether or not considered to be related to the medicinal product. An AE includes, but is not limited to: an exacerbation of a pre-existing illness; an increase in frequency or intensity of a pre-existing episodic event or condition; a condition detected or diagnosed after study drug administration even though it may have been present prior to the start of the study; and continuously persistent disease or symptoms that were present at baseline and worsen following the start of the study. An AE generally does not include: medical or surgical procedures (e.g., surgery, endoscopy, tooth extraction, or transfusion); however, the condition that leads to the procedure is an adverse event; pre-existing diseases, conditions, or laboratory abnormalities present or detected at the start of the study that do not worsen; hospitalizations or procedures that are done for elective purposes not related to an untoward medical occurrence (e.g., hospitalizations for cosmetic or elective surgery or social/convenience admissions); the disease being studied or signs/symptoms associated with the disease unless more severe than expected for the individual's condition; and overdose of study drug without any clinical signs or symptoms.

A “serious adverse event” or (SAE) as used herein refers to any untoward medical occurrence at any dose including, but not limited to, that: a) is fatal; b) is life-threatening (defined as an immediate risk of death from the event as it occurred); c) results in persistent or significant disability or incapacity; d) requires in-patient hospitalization or prolongs an existing hospitalization (exception: Hospitalization for elective treatment of a pre-existing condition that did not worsen during the study is not considered an adverse event. Complications that occur during hospitalization are AEs and if a complication prolongs hospitalization, then the event is serious); e) is a congenital anomaly/birth defect in the offspring of an individual who received medication; or f) conditions not included in the above definitions that may jeopardize the individual or may require intervention to prevent one of the outcomes listed above unless clearly related to the individual's underlying disease. “Lack of efficacy” (progressive disease) not considered an AE or SAE. The signs and symptoms or clinical sequelae resulting from lack of efficacy should be reported if they fulfill the AE or SAE definitions.

The following definitions may be used to evaluate response based on target lesions: “complete response” or “GR” refers to disappearance of all target lesions; “partial response” or “PR” refers to at least a 30% decrease in the sum of the longest diameters (SLD) of target lesions, taking as reference the baseline SLD; “stable disease” or “SD” refers to neither sufficient shrinkage of target lesions to qualify for PR, nor sufficient increase to qualify for PD, taking as reference the nadir SLD since the treatment started; and “progressive disease” or “PD” refers to at least a 20% increase in the SLD of target lesions, taking as reference the nadir SLD recorded since the treatment started, or, the presence of one or more new lesions.

The following definitions of response assessments may be used to evaluate a non-target lesion: “complete response” or “GR” refers to disappearance of all non-target lesions; “stable disease” or “SD” refers to the persistence of one or more non-target lesions not qualifying for GR or PD; and “progressive disease” or “PD” refers to the “unequivocal progression” of existing non-target lesion(s) or appearance of one or more new lesion(s) is considered progressive disease (if PD for the individual is to be assessed for a time point based solely on the progression of non-target lesion(s), then additional criteria are required to be fulfilled.

“Progression free survival” (PFS) indicates the length of time during and after treatment that the cancer does not grow. Progression-free survival includes the amount of time individuals have experienced a complete response or a partial response, as well as the amount of time individuals have experienced stable disease.

A “complete response” (GR) to a therapy defines individuals with evaluable but non-measurable disease, whose tumor and all evidence of disease had disappeared.

A “partial response” (PR) to a therapy defines individuals with anything less than complete response were simply categorized as demonstrating partial response.

“Stable disease” (SD) indicates that the individual is stable.

“Correlate” or “correlating” is meant comparing, in any way, the performance and/or results of a first analysis or protocol with the performance and/or results of a second analysis or protocol. For example one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

“Predicting” or “prediction” is used herein to refer to the likelihood that an individual is likely to respond either favorably or unfavorably to a treatment regimen.

As used herein, “at the time of starting treatment” or “baseline” refers to the time period at or prior to the first exposure to the treatment.

A method of “aiding assessment” as used herein refers to methods that assist in making a clinical determination and may or may not be conclusive with respect to the assessment.

“Likely to respond” or “responsiveness” as used herein refers to any kind of improvement or positive response either clinical or non-clinical selected from, but not limited to, measurable reduction in tumor size or evidence of disease or disease progression, complete response, partial response, stable disease, increase or elongation of progression free survival, or increase or elongation of overall survival.

As used herein, “sample” refers to a composition which contains a molecule which is to be characterized and/or identified, for example, based on physical, biochemical, chemical, physiological, and/or genetic characteristics.

“Cells,” as used herein, is understood to refer not only to the particular individual but to the progeny or potential progeny of such a cell. Because certain modifications may occur in succeeding generations due to either mutation or environmental influences, such progeny may not, in fact, be identical to the parent cell, but are still included within the scope of the term as used herein.

Level of a GR measured “before or upon initiation of treatment” is level of a GR measured in an individual before the individual receives the first administration of a treatment modality described herein.

An individual who “may be suitable”, which includes an individual who is “suitable” for treatments) described herein, is an individual who is more likely than not to benefit from administration of said treatments. Conversely, an individual who “may not be suitable” or “may be unsuitable”, which includes an individual who is “unsuitable” for treatment(s) described herein, is an individual who is more likely than not to fail to benefit from administration of said treatments.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

The term “about X-Y” used herein has the same meaning as “about X to about Y.”

As used herein and in the appended claims, the singular forms “a,” “or,” and “the” include plural referents unless the context clearly dictates otherwise.

As is apparent to one skilled in the art, an individual assessed, selected for, and/or receiving treatment is an individual in need of such activities.

Methods of Treating Cancer Based on Level of Glucocorticoid and/or its Receptor

The present application in one aspect provides methods of treating cancer based on level of glucocorticoid receptors (“GR”) or glucocorticoids (“GC”, such as cortisol),

In some embodiments, there is provided a method of treating an individual having a cancer characterized by a high level of glucocorticoid receptor (GR), comprising administering to the individual an effective amount of a composition comprising a taxane (for example a cremophor-free formulation of taxane). In some embodiments, there is provided a method of treating an individual having a cancer characterized by a high level of glucocorticoid receptor (GR), comprising administering to the individual an effective amount of a composition comprising nanoparticles comprising a taxane (such as paclitaxel). In some embodiments, there is provided a method of treating an individual having a cancer characterized by a high level of glucocorticoid receptor (GR), comprising administering to the individual an effective amount of a composition comprising nanoparticles comprising a taxane (such as paclitaxel) and an albumin. In some embodiments, there is provided a method of treating an individual having a cancer characterized by a high level of glucocorticoid receptor (GR), comprising administering to the individual an effective amount of a composition comprising nanoparticles comprising paclitaxel coated with albumin (including nanoparticles having an average diameter of no greater than about 200 nm). In some embodiments, there is provided a method of treating an individual having a cancer characterized by a high level of glucocorticoid receptor (GR), comprising administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized, by a high level of GR expression. In some embodiments, the individual is characterized by a high level of GR activity. In some embodiments, the individual is characterized by a high level of GR expression and a high level of GR activity. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined (e.g., high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer characterized by a high level of glucocorticoid (GC, such as cortisol), comprising administering to the individual an effective amount of a composition comprising a taxane (for example a cremophor-free formulation of taxane). In some embodiments, there is provided a method of treating an individual having a cancer characterized by a high level of glucocorticoid (GC, such as cortisol), comprising administering to the individual an effective amount of a composition comprising nanoparticles comprising a taxane (such as paclitaxel). In some embodiments, there is provided a method of treating an individual having a cancer characterized by a high level of glucocorticoid (GC, such as cortisol), comprising administering to the individual an effective amount of a composition comprising nanoparticles comprising a taxane (such as paclitaxel) and an albumin. In some embodiments, there is provided a method of treating an individual having a cancer characterized by a high level of glucocorticoid (GC, such as cortisol), comprising administering to the individual an effective amount of a composition comprising nanoparticles comprising paclitaxel coated with albumin (including nanoparticles having an average diameter of no greater than about 200 nm). In some embodiments, there is provided a method of treating an individual having a cancer characterized by a high level of glucocorticoid (GC, such as cortisol), comprising administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GC (such as cortisol) secretion. In some embodiments, the individual is characterized by a high level of GC (such as cortisol) activity. In some embodiments, the individual is characterized by a high level of GC (such as cortisol) secretion and a high level of GC (such as cortisol) activity. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer characterized by a high level of GR and a high level of GC (such as cortisol), comprising administering to the individual an effective amount of a composition comprising a taxane (for example a cremophor-free formulation of taxane). In some embodiments, there is provided a method of treating an individual having a cancer characterized by a high level of GR and a high level of GC (such as cortisol), comprising administering to the individual an effective amount of a composition comprising nanoparticles comprising a taxane (such as paclitaxel). In some embodiments, there is provided a method of treating an individual having a cancer characterized by a high level of GR and a high level of GC (such as cortisol), comprising administering to the individual an effective amount of a composition comprising nanoparticles comprising a taxane (such as paclitaxel) and an albumin. In some embodiments, there is provided a method of treating an individual having a cancer characterized by a high level of GR and a high level of GC (such as cortisol), comprising administering to the individual an effective amount of a composition comprising nanoparticles comprising paclitaxel coated with albumin (including nanoparticles having an average diameter of no greater than about 200 nm). In some embodiments, there is provided a method of treating an individual having a cancer characterized by a high level of GR and a high level of GC (such as cortisol), comprising administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GR expression and a high level of GC (such as cortisol) secretion. In some embodiments, the individual is characterized by a high level of GR activity and a high level of GC (such as cortisol) activity. In some embodiments, the individual is characterized by a high level of GR activity and a high level of GC (such as cortisol) secretion. In some embodiments, the individual is characterized by a high level of GR expression and a high level of GC (such as cortisol) activity. In some embodiments, the individual is characterized by a high level of GR in the tumor and a high level of GC (such as cortisol) in the blood. In some embodiments, the level of GR expression is based on protein expression. In some embodiments, the level of GR expression is based on mRNA level. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer, comprising administering to the individual an effective amount of a composition comprising a taxane (for example a cremophor-free, formulation of taxane), wherein a high level of GR is used as a basis for selecting the individual for treatment. In some embodiments, there is provided a method of treating an individual having a cancer, comprising administering to the individual an effective amount of a composition comprising nanoparticles comprising a taxane (such as paclitaxel), wherein a high level of GR is used as a basis for selecting the individual for treatment. In some embodiments, there is provided a method of treating an individual having a cancer, comprising administering to the individual an effective amount of a composition comprising nanoparticles comprising a taxane (such as paclitaxel) and an albumin, wherein a high level of GR is used as a basis for selecting the individual for treatment. In some embodiments, there is provided a method of treating an individual having a cancer, comprising administering to the individual an effective amount of a composition comprising nanoparticles comprising paclitaxel coated with albumin (including nanoparticles having an average diameter of no greater than about 200 nm), wherein a high level of GR is used as a basis for selecting the individual for treatment. In sonic embodiments, there is provided a method of treating individual having a cancer, comprising administering to the individual an effective amount of Nab-paclitaxel (for example about 5 mg/ml Nab-paclitaxel), wherein a high level of GR is used as a basis for selecting the individual for treatment. In some embodiments, the cancer is pancreatic cancer. In some embodiments, the method does not require premedication. In some embodiments, a high level of GR expression is used as a basis for selecting the individual for treatment. In some embodiments, a high level of GR activity is used as a basis for selecting the individual for treatment. In some embodiments, a high level of GR expression and a high level of GR activity are used as a basis for selecting the individual for treatment. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined (e.g., high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer, comprising administering to the individual an effective amount of a composition comprising a taxane (for example a cremophor-free formulation of taxane), wherein a high level of GC (such as cortisol) is used as a basis for selecting the individual for treatment. In some embodiments, there is provided a method of treating an individual having a cancer, comprising administering to the individual an effective amount of a composition comprising nanoparticles comprising a taxane (such as paclitaxel), wherein a high level of GC (such as cortisol) is used as a basis for selecting the individual for treatment. In some embodiments, there is provided a method of treating an individual having a cancer, comprising administering to the individual an effective amount of a composition comprising nanoparticles comprising a taxane (such as paclitaxel) and an albumin, wherein a high level of GC (such as cortisol) is used as a basis for selecting the individual for treatment. In some embodiments, there is provided a method of treating an individual having a cancer, comprising administering to the individual an effective amount of a composition comprising nanoparticles comprising paclitaxel coated with albumin (including nanoparticles having an average diameter of no greater than about 200 nm), wherein a high level of GC (such as cortisol) is used as a basis for selecting the individual for treatment. In some embodiments, there is provided a method of treating an individual having a cancer, comprising administering to the individual an effective amount of Nab-paclitaxel (for example about 5 mg/ml Nab-paclitaxel), wherein a high level of GC (such as cortisol) is used as a basis for selecting the individual for treatment. In some embodiments, the cancer is pancreatic cancer. In some embodiments, the method does not require premedication. In some embodiments, a high level of GC (such as cortisol) secretion is used as a basis for selecting the individual for treatment. In some embodiments, a high level of GC (such as cortisol) activity is used as a basis for selecting the individual for treatment. In some embodiments, a high level of GC (such as cortisol) secretion and a high level of GC (such as cortisol) activity are used as a basis for selecting the individual for treatment. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or law) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some, embodiments, the method further comprises administering to the individual another agent (such as an agent that inhibits GR expression or activity).

In some embodiments, there is provided a method of treating an individual having a cancer, comprising administering to the individual an effective amount of a composition comprising a taxane (for example a cremophor-free formulation of taxane), wherein a high level of GR and a high level of GC (such as cortisol) are used as a basis for selecting the individual for treatment. In some embodiments, there is provided a method of treating an individual having a cancer, comprising administering to the individual an effective amount of a composition comprising nanoparticles comprising a taxane (such as paclitaxel), wherein a high level of GR and a high level of GC (such as cortisol) are used as a basis for selecting the individual for treatment. In some embodiments, there is provided a method of treating an individual having a cancer, comprising administering to the individual an effective amount of a composition comprising nanoparticles comprising a taxane (such as paclitaxel) and an albumin, wherein a high level of GR and a high level of GC (such as cortisol) are used as a basis for selecting the individual for treatment. In some embodiments, there is provided a method of treating an individual having a cancer, comprising administering to the individual an effective amount of a composition comprising nanoparticles comprising paclitaxel coated with albumin (including nanoparticles having an average diameter of no greater than about 200 nm), wherein a high level of GR and a high level of GC (such as cortisol) are used as a basis for selecting the individual for treatment. In some embodiments, there is provided a method of treating an individual having a cancer, comprising administering to the individual an effective amount of Nab-paclitaxel (for example about 5 mg/ml Nab-paclitaxel), wherein a high level of GR and a high level of GC (such as cortisol) are used as a basis for selecting the individual for treatment. In some embodiments, the cancer is pancreatic cancer. In some embodiments, the method does not require premedication. In some embodiments, a high level of GR expression and a high level of GC (such as cortisol) secretion are used as a basis for selecting the individual for treatment. In some embodiments, a high level of GR activity and a high level of GC (such as cortisol) activity are used as a basis for selecting the individual for treatment. In some embodiments, a high level of GR activity and a high level of GC (such as cortisol) secretion are used as a basis for selecting the individual for treatment. In some embodiments, a high level of GR expression and a high level of GC (such as cortisol) activity are used as a basis for selecting the individual for treatment. In some embodiments, a high level of GR in the tumor and a high level of GC (such as cortisol) in the blood is used as a basis for selecting the individual for treatment. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva), in some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of selecting (including identifying) an individual having cancer for treating with a composition comprising a taxane (for example a cremophor-free formulation of taxane), wherein the method comprises determining the level of GR in the individual, wherein the individual is selected for treatment if the individual has a high level of GR. In some embodiments, there is provided a method of selecting (including identifying) an individual having cancer for treating with a composition comprising nanoparticles comprising a taxane (such as paclitaxel), wherein the method comprises determining the level of GR in the individual, wherein the individual is selected for treatment if the individual has a high level of GR. In some embodiments, there is provided a method of selecting (including identifying) an individual having cancer for treating with a composition comprising nanoparticles comprising a taxane (such as paclitaxel) and an albumin, wherein the method comprises determining the level of GR in the individual, wherein the individual is selected for treatment if the individual has a high level of GR. In some embodiments, there is provided a method of selecting (including identifying) an individual having cancer for treating with a composition comprising nanoparticles comprising paclitaxel coated with albumin (including nanoparticles having an average diameter of no greater than about 200 nm), wherein the method comprises determining the level of GR in the individual, wherein the individual is selected for treatment if the individual has a high level of GR. In some embodiments, there is provided a method of selecting (including identifying) an individual having cancer for treating with Nab-paclitaxel (for example about 5 mg/ml Nab-paclitaxel), wherein the method comprises determining the level of GR in the individual, wherein the individual is selected for treatment if the individual has a high level of GR. In some embodiments, the cancer is pancreatic cancer, in some embodiments, the method does not require premedication. In some embodiments, the individual is selected for treatment if the individual has a high level of GR expression. In some embodiments, the individual is selected for treatment if the individual has a high level of GR activity. In some embodiments, the individual is selected for treatment if the individual has a high level of GR expression and a high level of GR activity. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined (e.g., high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein). In some embodiments, the treatment further comprises administering to the individual another agent (such as an agent that inhibits GR expression or activity).

In some embodiments, there is provided a method of selecting (including identifying) an individual having cancer for treating with a composition comprising a taxane (for example a cremophor-free formulation of taxane), wherein the method comprises determining the level of GC (such as cortisol) in the individual, wherein the individual is selected for treatment if the individual has a high level of GC (such as cortisol). In some embodiments, there is provided a method of selecting (including identifying) an individual having cancer for treating with a composition comprising nanoparticles comprising a taxane (such as paclitaxel), wherein the method comprises determining the level of GC (such as cortisol) in the individual, wherein the individual is selected for treatment if the individual has a high level of GC (such as cortisol). In some embodiments, there is provided a method of selecting (including identifying) an individual having cancer for treating with a composition comprising nanoparticles comprising a taxane and an albumin, wherein the method comprises determining the level of GC (such as cortisol) in the individual, wherein the individual is selected for treatment if the individual has a high level of GC (such as cortisol). In some embodiments, there is provided a method of selecting (including identifying) an individual having cancer for treating with a composition comprising nanoparticles comprising paclitaxel coated with albumin (including nanoparticles having an average diameter of no greater than about 200 nm), wherein the method comprises determining the level of GC (such as cortisol) in the individual, wherein the individual is selected for treatment if the individual has a high level of GC (such as cortisol). In some embodiments, there is provided a method of selecting (including identifying) an individual having cancer for treating with Nab-paclitaxel (for example about 5 mg/ml Nab-paclitaxel), wherein the method comprises determining the level of GC (such as cortisol) in the individual, wherein the individual is selected for treatment if the individual has a high level of GC (such as cortisol). In some embodiments, the cancer is pancreatic cancer. In some embodiments, the method does not require premedication. In some embodiments, the individual is selected for treatment if the individual has a high level of GC (such as cortisol) secretion. In some embodiments, the individual is selected for treatment if the individual has a high level of GC (such as cortisol) activity. In some embodiments, the individual is selected for treatment if the individual has a high level of GC (such as cortisol) secretion and a high level of GC (such as cortisol) activity. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the treatment further comprises administering to the individual another agent (such as an agent that inhibits GR expression or activity).

In some embodiments, there is provided a method of selecting (including identifying) an individual having cancer for treating with a composition comprising a taxane (for example a cremophor-free formulation of taxane), wherein the method comprises determining the level of GR and GC (such as cortisol) in the individual, wherein the individual is selected for treatment if the individual has a high level of GR and a high level of GC (such as cortisol). In some embodiments, there is provided a method of selecting (including identifying) an individual having cancer for treating with a composition comprising nanoparticles comprising a taxane (such as paclitaxel), wherein the method comprises determining the level of GR and GC (such as cortisol) in the individual, wherein the individual is selected for treatment if the individual has a high level of GR and a high level of GC (such as cortisol). In some embodiments, there is provided a method of selecting (including identifying) an individual having cancer for treating with a composition comprising nanoparticles comprising a taxane (such as paclitaxel) and an albumin, wherein the method comprises determining the level of GR and GC (such as cortisol) in the individual, wherein the individual is selected for treatment if the individual has a high level of GR and a high level of GC (such as cortisol). In some embodiments, there is provided a method of selecting (including identifying) an individual having cancer for treating with a composition comprising nanoparticles comprising paclitaxel coated with albumin (including nanoparticles having an average diameter of no greater than about 200 nm), wherein the method comprises determining the level of GR and GC (such as cortisol) in the individual, wherein the individual is selected for treatment if the individual has a high level of GR and a high level of GC (such as cortisol). In some embodiments, there is provided a method of selecting (including identifying) an individual having cancer for treating with Nab-paclitaxel (for example about 5 mg/ml Nab-paclitaxel), wherein the method comprises determining the level of GR and GC (such as cortisol) in the individual, wherein the individual is selected for treatment if the individual has a high level of GR and a high level of GC (such as cortisol). In some embodiments, the cancer is pancreatic cancer. In some embodiments, the method does not require premedication. In some embodiments, the individual is selected for treatment if the individual has a high level of GR expression and a high level of GC (such as cortisol) secretion. In some embodiments, the individual is selected for treatment if the individual has a high level of GR activity and a high level of GC (such as cortisol) activity. In some embodiments, the individual is selected for treatment if the individual has a high level of GR activity and a high level of GC (such as cortisol) secretion. In some embodiments, the individual is selected for treatment if the individual has a high level of GR expression and a high level of GC (such as cortisol) activity. In some embodiments, the individual is selected for treatment if the individual has a high level of GR in the tumor and a high level of GC (such as cortisol) in the blood. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the treatment further comprises administering to the individual another agent (such as an agent that inhibits GR expression or activity).

In some embodiments, there is provided a method of treating an individual having a cancer characterized by a high level of glucocorticoid receptor (GR), comprising a) determining the level of GR in the individual, and b) administering to the individual an effective amount of a composition comprising a taxane (for example a cremophor-free formulation of taxane). In some embodiments, there is provided a method of treating an individual having a cancer characterized by a high level of glucocorticoid receptor (GR), comprising a) determining the level of GR in the individual, and b) administering to the individual an effective amount of a composition comprising nanoparticles comprising a taxane (such as paclitaxel). In some embodiments, there is provided a method of treating an individual having a cancer characterized by a high level of glucocorticoid receptor (GR), comprising a) determining the level of GR in the individual, and b) administering to the individual an effective amount of a composition comprising nanoparticles comprising a taxane (such as paclitaxel) and an albumin. In some embodiments, there is provided a method of treating an individual having a cancer characterized by a high level of glucocorticoid receptor (GR), comprising a) determining the level of GR in the individual, and b) administering to the individual an effective amount of a composition comprising nanoparticles comprising paclitaxel coated with albumin (including nanoparticles having an average diameter of no greater than about 200 nm). In some embodiments, there is provided a method of treating an individual having a cancer characterized by a high level of glucocorticoid receptor (GR), comprising a) determining the level of GR in the individual, and h) administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GR expression. In some embodiments, the individual is characterized by a high level of GR activity. In some embodiments, the individual is characterized by a high level of GR expression and a high level of GR activity. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined (e.g., high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer characterized by a high level of glucocorticoid (GC, such as cortisol), comprising a) determining the level of GC such as cortisol) in the individual, and b) administering to the individual an effective amount of a composition comprising a taxane (for example a cremophor-free formulation of taxane). In some embodiments, there is provided a method of treating an individual having a cancer characterized by a high level of glucocorticoid (GC, such as cortisol), comprising; a) determining the level of GC (such as cortisol) in the individual, and b) administering to the individual an effective amount of a composition comprising nanoparticles comprising a taxane (such as paclitaxel). In some embodiments, there is provided a method of treating an individual having a cancer characterized by a high level of glucocorticoid (GC, such as cortisol), comprising a) determining the level of GC (such as cortisol) in the individual, and b) administering to the individual an effective amount of a composition comprising nanoparticles comprising a taxane (such as paclitaxel) and an albumin. In some embodiments, the individual is characterized by a high level of GC (such as cortisol) secretion. In some embodiments, there is provided a method of treating an individual having a cancer characterized by a high level of glucocorticoid (GC, such as cortisol), comprising a) determining the level of GC (such as cortisol) in the individual, and b) administering to the individual an effective amount of a composition comprising nanoparticles comprising paclitaxel coated with albumin (including nanoparticles having an average diameter of no greater than about 200 nm). In some embodiments, the individual is characterized by a high level of GC (such as cortisol) secretion. In some embodiments, there is provided a method of treating an individual having a cancer characterized by a high level of glucocorticoid (GC, such as cortisol), comprising a) determining the level of GC (such as cortisol) in the individual, and b) administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GC (such as cortisol) secretion. In some embodiments, the individual is characterized by a high level of GC (such as cortisol) activity. In some embodiments, the individual is characterized by a high level of GC (such as cortisol) secretion and a high level of GC (such as cortisol) activity. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer characterized by a high level of glucocorticoid receptor (GR), comprising a) determining the level of GR and the level of GC (such as cortisol) in the individual, and b) administering to the individual an effective amount of a composition comprising a taxane (for example a cremophor-free formulation of taxane). In some embodiments, there is provided a method of treating an individual having a cancer characterized by a high level of glucocorticoid receptor (GR), comprising a) determining the level of GR and the level of GC (such as cortisol) in the individual, and b) administering to the individual an effective amount of a composition comprising nanoparticles comprising a taxane (such as paclitaxel). In some embodiments, there is provided a method of treating an individual having a cancer characterized by a high level of glucocorticoid receptor (GR), comprising a) determining the level of GR and the level of GC (such as cortisol) in the individual, and b) administering to the individual an effective amount of a composition comprising nanoparticles comprising a taxane (such as paclitaxel) and an albumin. In some embodiments, there is provided a method of treating an individual having a cancer characterized by a high level of glucocorticoid receptor (GR), comprising a) determining the level of GR and the level of GC (such as cortisol) in the individual, and b) administering to the individual an effective amount of a composition comprising nanoparticles comprising paclitaxel coated with albumin (including nanoparticles having an average diameter of no greater than about 200 nm). In some embodiments, there is provided a method of treating an individual having a cancer characterized by a high level of glucocorticoid receptor (GR), comprising a) determining the level of GR and the level of GC (such as cortisol) in the individual, and 2) administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GR expression and a high level of GC (such as cortisol) secretion. In some embodiments, the individual is characterized by a high level of GR activity and a high level of GC (such as cortisol) activity. In some embodiments, the individual is characterized by a high level of GR activity and a high level of GC (such as cortisol) secretion. In some embodiments, the individual is characterized by a high level of GR in the tumor and a high level of GC (such as cortisol) in the blood. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer, comprising a) selecting the individual for treatment based on a high level of GR in the individual, and b) administering to the individual an effective amount of a composition comprising a taxane (for example a cremophor-free formulation of taxane). In some embodiments, there is provided a method of treating an individual having a cancer, comprising a) selecting the individual for treatment based on a high level of GR in the individual, and b) administering to the individual an effective amount of a composition comprising nanoparticles comprising a taxane (such as paclitaxel). In some embodiments, there is provided a method of treating an individual having a cancer, comprising a) selecting the individual for treatment based on a high level of GR in the individual, and b) administering to the individual an effective amount of a composition comprising nanoparticles comprising a taxane (such as paclitaxel) and an albumin. In some embodiments, there is provided a method of treating an individual having a cancer, comprising a) selecting the individual for treatment based on a high level of GR in the individual, and 2) administering to the individual an effective amount of a composition comprising nanoparticles comprising paclitaxel coated with albumin (including nanoparticles having an average diameter of no greater than about 200 nm). In some embodiments, there is provided a method of treating an individual having a cancer, comprising a) selecting the individual for treatment based on a high level of GR in the individual, and 2) administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GR expression. In some embodiments, the individual is characterized by a high level of GR activity. In some embodiments, the individual is characterized by a high level of GR expression and a high level of GR activity. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer, comprising a) selecting the individual for treatment based on a high level of GC (such as cortisol) in the individual, and b) administering to the individual an effective amount of a composition comprising a taxane (for example a cremophor-free formulation of taxane). In some embodiments, there is provided a method of treating an individual having a cancer, comprising a) selecting the individual for treatment based on a high level of GC (such as cortisol) in the individual, and b) administering to the individual an effective amount of a composition comprising nanoparticles comprising a taxane (such as paclitaxel). In some embodiments, there is provided a method of treating an individual having a cancer, comprising a) selecting the individual for treatment based on a high level of GC (such as cortisol) in the individual, and b) administering to the individual an effective amount of a composition comprising nanoparticles comprising a taxane (such as paclitaxel) and an albumin. In some embodiments, the individual is characterized by a high level of GC (such as cortisol) secretion. In some embodiments, there is provided a method of treating an individual having a cancer, comprising a) selecting the individual for treatment based on a high level of GC (such as cortisol) in the individual, and b) administering to the individual an effective amount of a composition comprising nanoparticles comprising paclitaxel coated with albumin including nanoparticles having an average diameter of no greater than about 200 nm). In some embodiments, the individual is characterized by a high level of GC (such as cortisol) secretion. In some embodiments, there is provided a method of treating an individual having a cancer, comprising a) selecting the individual for treatment based on a high level of GC (such as cortisol) in the individual, and b) administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GC (such as cortisol) secretion. In some embodiments, the individual is characterized by a high level of GC (such as cortisol) activity. In some embodiments, the individual is characterized by a high level of GC (such as cortisol) secretion and a high level of GC (such as cortisol) activity. In some embodiments, the level of GC secretion is based on endogenous GC secretion (such as cortisol secretion). In some embodiments, the level of GC activity is based on endogenous GC (such as cortisol) activity. In some embodiments, the level of GC secretion is based on free GC in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer, comprising a) selecting the individual for treatment based on a high level of GR and a high level of GC (such as cortisol) in the individual, and b) administering to the individual an effective amount of a composition comprising a taxane (for example a cremophor-free formulation of taxane). In some embodiments, there is provided a method of treating an individual having a cancer, comprising a) selecting the individual for treatment based on a high level of GR and a high level of GC (such as cortisol) in the individual, and b) administering to the individual an effective amount of a composition comprising nanoparticles comprising a taxane (such as paclitaxel). In some embodiments, there is provided a method of treating an individual having a cancer, comprising a) selecting the individual for treatment based on a high level of GR and a high level of GC (such as cortisol) in the individual, and b) administering to the individual an effective amount of a composition comprising nanoparticles comprising a taxane (such as paclitaxel) and an albumin. In some embodiments, there is provided a method of treating an individual having a cancer, comprising a) selecting the individual for treatment based on a high level of GR and a high level of GC (such as cortisol) in the individual, and b) administering to the individual an effective amount of a composition comprising nanoparticles comprising paclitaxel coated with albumin (including nanoparticles having an average diameter of no greater than about 200 nm). In some embodiments, there is provided a method of treating an individual having a cancer, comprising a) selecting the individual for treatment based on a high level of GR and a high level of GC (such as cortisol) in the individual, and 2) administering to the individual an effective amount of Nab-paclitaxel (for example about 5 Nab-paclitaxel). In some embodiments, the cancer is pancreatic cancer. In some embodiments, the method does not require premedication. In some embodiments, the individual is characterized by a high level of GR expression and a high level of GC (such as cortisol) secretion. In some embodiments, the individual is characterized by a high level of GR activity and a high level of GC (such as cortisol) activity. In some embodiments, the individual is characterized by a high level of GR activity and a high level of GC (such as cortisol) secretion. In some embodiments, the individual is characterized by a high level of GR expression and a high level of GC (such as cortisol) activity. In some embodiments, the individual is characterized by a high level of GR in the tumor and a high level of GC (such as cortisol) in the blood. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer, comprising a) determining the level of GR in the individual; b) selecting the individual for treatment based on a high level of GR in the individual, and c) administering to the individual an effective amount of a composition comprising a taxane (for example a cremophor-free formulation of taxane). In some embodiments, there is provided a method of treating an individual having a cancer, comprising a) determining the level of GR in the individual; b) selecting the individual for treatment based on a high level of GR in the individual, and c) administering to the individual an effective amount of a composition comprising nanoparticles comprising a taxane (such as paclitaxel). In some embodiments, there is provided a method of treating an individual having a cancer, comprising a) determining the level of GR in the individual; b) selecting the individual for treatment based on a high level of GR in the individual, and c) administering to the individual an effective amount of a composition comprising nanoparticles comprising a taxane (such as paclitaxel) and an albumin. In some embodiments, there is provided a method of treating an individual having a cancer, comprising a) determining the level of GR in the individual; b) selecting the individual for treatment based on a high level of GR in the individual; and c) administering to the individual an effective amount of a composition comprising nanoparticles comprising paclitaxel coated with albumin (including nanoparticles having an average diameter of no greater than about 200 nm). In some embodiments, there is provided a method of treating an individual having a cancer, comprising a) determining the level of GR in the individual; b) selecting the individual for treatment based on a high level of GR in the individual, and c) administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GR expression. In some embodiments, the individual is characterized by a high level of GR activity. In some embodiments, the individual is characterized by a high level of GR expression and a high level of GR activity. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined (e.g., high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer, comprising a) determining the level of GC (such as cortisol) in the individual; b) selecting the individual for treatment based on a high level of GC (such as cortisol) in the individual, and c) administering to the individual an effective amount of a composition comprising a taxane (for example a cremophor-free formulation of taxane). In some embodiments, there is provided a method of treating an individual having a cancer, comprising a) determining the level of GC (such as cortisol) in the individual; b) selecting the individual for treatment based on a high level of GC (such as cortisol) in the individual, and c) administering to the individual an effective amount of a composition comprising nanoparticles comprising a taxane (such as paclitaxel). In some embodiments, there is provided a method of treating an individual having a cancer, comprising a) determining the level of GC (such as cortisol) in the individual; b) selecting the individual for treatment based on a high level of GC (such as cortisol) in the individual, and c) administering to the individual an effective amount of a composition comprising nanoparticles comprising a taxane (such as paclitaxel) and an albumin. In some embodiments, the individual is characterized by a high level of GC (such as cortisol) secretion. In some embodiments, there is provided a method of treating an individual having a cancer, comprising a) determining the level of GC (such as cortisol) in the individual; b) selecting the individual for treatment based on a high level of GC (such as cortisol) in the individual, and c) administering to the individual an effective amount of a composition comprising nanoparticles comprising paclitaxel coated with albumin (including nanoparticles having an average diameter of no greater than about 200 nm). In some embodiments, the individual is characterized by a high level of GC (such as cortisol) secretion. In some embodiments, there is provided a method of treating an individual having a cancer, comprising a) determining the level of GC (such as cortisol) in the individual; b) selecting the individual for treatment based on a high level of GC (such as cortisol) in the individual, and c) administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GC (such as cortisol) secretion. In some embodiments, the individual is characterized by a high level of GC (such as cortisol) activity. In some embodiments, the individual is characterized by a high level of GC (such as cortisol) secretion and a high level of GC (such as cortisol) activity. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein). In some embodiments, the method further comprises administering to the individual another agent (such as an agent that inhibits GR expression or activity).

In some embodiments, there is provided a method of treating an individual having a cancer, comprising a) determining the levels of GR and GC (such as cortisol) in the individual; b) selecting the individual for treatment based on a high level of GR and a high level of GC (such as cortisol) in the individual, and c) administering to the individual an effective amount of a composition comprising a taxane (for example a cremophor-free formulation of taxane). In some embodiments, there is provided a method of treating an individual having a cancer, comprising a) determining the levels of GR and GC (such as cortisol) in the individual; b) selecting the individual for treatment based on a high level of GR and a high level of GC (such as cortisol) in the individual, and c) administering to the individual an effective amount of a composition comprising nanoparticles comprising a taxane (such as paclitaxel). In some embodiments, there is provided a method of treating an individual having a cancer, comprising a) determining the levels of GR and GC (such as cortisol) in the individual; b) selecting the individual for treatment based on a high level of GR and a high level of GC (such as cortisol) in the individual, and c) administering to the individual an effective amount of a composition comprising nanoparticles comprising a taxane (such as paclitaxel) and an albumin. In some embodiments, there is provided a method of treating an individual having a cancer, comprising a) determining the levels of GR and GC (such as cortisol) in the individual; b) selecting the individual for treatment based on a high level of GR and a high level of GC (such as cortisol) in the individual, and c) administering to the individual an effective amount of a composition comprising nanoparticles comprising paclitaxel coated with albumin (including nanoparticles having an average diameter of no greater than about 200 nm). In some embodiments, there is provided a method of treating an individual having a cancer, comprising a) determining the levels of GR and GC (such as cortisol) in the individual; b) selecting the individual for treatment based on a high level of GR and a high level of GC (such as cortisol) in the individual, and c) administering to the individual an effective amount of Nab-paclitaxel (for example about 5 mg/ml Nab-paclitaxel). In some embodiments, the cancer is pancreatic cancer. In some embodiments, the method does not require premedication. In some embodiments, the individual is characterized by a high level of GR expression and a high level of GC (such as cortisol) secretion. In some embodiments, the individual is characterized by a high level of GR activity and a high level of GC (such as cortisol) activity. In some embodiments, the individual is characterized by a high level of GR activity and a high level of GC (such as cortisol) secretion. In some embodiments, the individual is characterized by a high level of GR expression and a high level of GC (such as cortisol) activity. In some embodiments, the individual is characterized by a high level of GR in the tumor and a high level of GC (such as cortisol) in the blood. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of GR or GC (such as cortisol) with a control. In some embodiments, the level is determined (e.g., high or low) based on a scoring system (such as any of the scoring systems described herein). In some embodiments, the method further comprises administering to the individual another agent (such as an agent that inhibits GR expression or activity).

Methods are also provided herein of assessing whether an individual with cancer will likely respond to treatment, wherein the treatment comprises an effective amount of a composition comprising a taxane (such as a composition comprising nanoparticles comprising a taxane (e.g., paclitaxel) and an albumin), the method comprising assessing the levels of GR and/or GC (such as cortisol), wherein a high level of a GR and/or GC (such as cortisol) indicates that the individual will likely be responsive to the treatment. In some embodiments, the method further comprises administering i) an effective amount of a composition comprising a taxane (such as a composition comprising nanoparticles comprising a taxane (e.g., paclitaxel) and an albumin).

Methods are also provided herein of aiding assessment of whether an individual with cancer will likely respond to or is suitable for treatment, wherein the treatment comprises an effective amount of a composition comprising a taxane (such as a composition comprising nanoparticles comprising a taxane (e.g., paclitaxel) and an albumin), the method comprising evaluating the levels of a GR and/or GC (such as cortisol), wherein a high level of the GR and/or GC (such as cortisol) indicates that the individual will likely be responsive to the treatment. In some embodiments, the method further comprises administering i) an effective amount of a composition comprising a taxane (such as a composition comprising nanoparticles comprising a taxane (e.g., paclitaxel) and an albumin).

In addition, methods are provided herein of identifying an individual with cancer likely to respond to treatment comprising an effective amount of a composition comprising a taxane (such as a composition comprising nanoparticles comprising a taxane (e.g., paclitaxel) and an albumin), the method comprising: (a) assessing the levels of a GR and/or GC (such as cortisol); and (b) identifying the individual having high level of a GR and/or GC (such as cortisol). In some embodiments, the method further comprises administering i) an effective amount of a composition comprising a taxane (such as a composition comprising nanoparticles comprising a taxane (e.g., paclitaxel) and an albumin). In some embodiments, the amount of taxane in the composition is determined based upon the level of the GR and/or GC (such as cortisol).

Also provided herein are methods of adjusting therapy treatment of an individual with cancer receiving an effective amount of a composition comprising a taxane (such as a composition comprising nanoparticles comprising a taxane (e.g., paclitaxel) and an albumin), the method comprising assessing the levels of a GR and/or GC (such as cortisol) in a sample isolated from the individual, and adjusting the therapy treatment based on the assessment. In some embodiments, the amount of the taxane is adjusted.

Provided herein are also methods for marketing a therapy comprising an effective amount of a composition comprising a taxane (such as a composition comprising nanoparticles comprising a taxane (e.g., paclitaxel) and an albumin) for use in a cancer individual subpopulation, the methods comprising informing a target audience about the use of the therapy for treating the individual subpopulation characterized by the individuals of such subpopulation having a sample which has high or low levels of a GR and/or GC (such as cortisol).

In some embodiments of any of the methods herein, the methods are predictive of and/or result in a measurable reduction in tumor size or evidence of disease or disease progression, complete response, partial response, stable disease, increase or elongation of progression free survival, or increase or elongation of overall survival. In some embodiments of any of the methods above, an individual is likely to respond to a taxane composition (such as a cremophor-free formulation of taxane, including Nab-paclitaxel), alone or in combination with an agent that reduces the expression, or activity of GR, or modulates the expression or activity of GR-responsive molecules, if the individual has a high GR and/or GC (such as cortisol) level, as evident by a measurable reduction in tumor size or evidence of disease or disease progression, complete response, partial response, stable disease, increase or elongation of progression free survival, increase or elongation of overall survival.

In some embodiments of any of the methods, there is provided a method of inhibiting cancer cell proliferation (such as tumor growth) in an individual, comprising administering to the individual an effective amount of a composition comprising a taxane (such as a composition comprising nanoparticles comprising a taxane (e.g., paclitaxel) and an albumin), wherein the individual is selected on the basis of a high GR and/or GC level. In some embodiments, at least about 10% (including for example at least about any of 20%, 30%, 40%, 60%, 70%, 80%, 90%, or 100%) cell proliferation is inhibited.

In some embodiments of any of the methods, there is provided a method of inhibiting tumor metastasis in an individual, comprising administering to the individual an effective amount of a composition comprising a taxane (such as a composition comprising nanoparticles comprising a taxane (e.g., paclitaxel) and an albumin), wherein the individual is selected on the basis of a high GR and/or GC level. In some embodiments, at least about 10% (including for example at least about any of 20%, 30%, 40%, 60%, 70%, 80%, 90%, or 100%) metastasis is inhibited. In some embodiments, method of inhibiting metastasis to lymph node is provided.

In some embodiments of any of the methods, there is provided a method of reducing tumor size in an individual, comprising administering to the individual an effective amount of a composition comprising a taxane (such as a composition comprising nanoparticles comprising a taxane (e.g., paclitaxel) and an albumin), wherein the individual is selected on the basis of a high GR and/or GC level. In some embodiments, the tumor size is reduced at least about 10% (including for example at least about any of 20%, 30%, 40%, 60%, 70%, 80%, 90%, or 100%).

In some embodiments of any of the methods, there is provided a method of prolonging progression-free survival of cancer in an individual, comprising administering to the individual an effective amount of a composition comprising a taxane (such as a composition comprising nanoparticles comprising a taxane (e.g., paclitaxel) and an albumin), wherein the individual is selected on the basis of a high GR and/or GC level. In some embodiments, the method prolongs the time to disease progression by at least any of 1, 2, 3, 4, 5, 6, 7, 8, 9, 10, 11, or 12 weeks.

In some embodiments of any of the methods, there is provided a method of prolonging survival of an individual having cancer, comprising administering to the individual an effective amount of a composition comprising a taxane (such as a composition comprising nanoparticles comprising a taxane (e.g., paclitaxel) and an albumin), wherein the individual is selected on the basis of a high GR and/or GC level. In some embodiments, the method prolongs the survival of the individual by at least any of 1, 2, 3, 4, 5, 6, 7, 8, 9, 10, 11, 12, 18, or 24 months.

In some embodiments of any of the methods, there is provided a method of reducing AEs and SAEs in an individual having cancer, comprising administering to the individual a composition comprising a taxane (such as a composition comprising nanoparticles comprising a taxane (e.g., paclitaxel) and an albumin), wherein the individual is selected on the basis of a high GR and/or GC level. In some embodiments of any of the methods described herein, the method is predictive of and/or results in an objective response (such as a partial response or complete response).

In some embodiments of any of the methods described herein, the method is predictive of and/or results in improved quality of life.

“High level of GR” and “high level of GC” refers to a GR or GC level that is above a control level. In some embodiments, the control level is median level of a control population, for example a population having the same cancer as the treated individual has. In some embodiments, the GR or GC level of the individual having a high level of GR or GC is at about any of 55%, 60%, 70%, 80%, 90%, 95%, 98%, or 99% percentile within the population. In some embodiments, the control level is a pre-determined threshold level. In some embodiments, the pre-determined threshold level is determined by cross-referencing GR levels of the control population to GR levels of the Cancer Genome Atlas (TCGA) cohort of the same cancer (such as same type of cancer) according to the Pan-Cancer analysis (The Cancer Genome Atlas Research Network et al. (2013) “The Cancer Genome Altas Pan-Cancer analysis project”, Nature Genetics 45:1113-1120, incorporated herein by reference in its entirety), wherein the pre-determined threshold level corresponds to the median GR level of the TGCA cohort of the same cancer. In some embodiments, the pre-determined threshold level corresponds to more than about any of 6, 7, 8, 9, 10, 11, 12, 13, or 14 relative expression units according to the Pan Cancer analysis of the TCGA cohort corresponding to the cancer of the individual. In some embodiments, the pre-determined threshold level of GR determined by a Western blot assay is about any of 2, 3, 4, 4.5, 5, 5.5, 6, 7, 8, 9, 10, 11, 12, 13, 14, or more, wherein the GR level of the individual is the GR protein level in a sample of the individual divided by the protein level of glyceraldehyde 3-phosphate dehydrogenase (GAPDH) in the same sample under the same Western blot assay conditions. In some embodiments, the pre-determined threshold level of GC (such as cortisol) is at least about any of 1.3, 1.5, 1.7, 2, 3, 4, 5, or more times that of a clinically accepted normal GC level in a standardized GC test (such as a cortisol blood test, or a 24-hour cortisol urine test).

The level of GR and/or GC can also be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. In some embodiments, the level of GR can also be useful for aiding assessment in any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits.

As used herein, “based upon” or “based on” include assessing, determining, or measuring the individual's characteristics as described herein (and preferably selecting an individual suitable for receiving treatment). When the level of GR or GC is used as a basis for selection, assessing (or aiding in assessing), measuring, or determining for methods of treatment as described herein, the level of GR or GC is measured before and/or during treatment, and the values obtained can be used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

In some embodiments of any of the methods, the treatment comprises administration of the composition comprising a taxane (such as a composition comprising nanoparticles comprising the taxane and the albumin) over less than about 50 minutes, such as less than about 40 minutes, less than about 30 minutes or about 30 to about 40 minutes. In some embodiments of any of the methods, the treatment comprises an amount (dose) of the composition comprising taxane at between about 50 mg/m² and about 300 mg/m² (including for example about 50 mg/m² to about 260 mg/m², about 100 mg/m² to about 150 mg/m², for example about 125 mg/m²). In some embodiments, the amount (dose) of the composition comprising the taxane is about 50 mg/m², about 75 mg/m², or about 100 mg/m², about 125 mg/m², or about 150 mg/m². In some embodiments of any of the methods, the treatment comprises administration of the composition comprising the taxane (such as the composition comprising nanoparticles comprising a taxane and an albumin) parenterally. In some embodiments of any of the methods, the treatment comprises administration of the composition comprising a taxane (such as a composition comprising nanoparticles comprising the taxane and the albumin) intravenously. In some embodiments of any of the methods, the treatment comprises administration of the composition comprising a taxane (such as a composition comprising nanoparticles comprising the taxane and the albumin) weekly or weekly, three out of four weeks. In some embodiments of any of the methods, the treatment comprises administration of the composition comprising a taxane (such as a composition comprising nanoparticles comprising the taxane and the albumin) without any premedication (for example steroid premedication) and/or without G-CSF prophylaxis.

In some embodiments, the individual is human. In some embodiments, the individual is a female. In some embodiments, the individual is a male. In some embodiments, the individual is under about 65 years old. In some embodiments, the individual is at least about 65 years old, at least about 70 years old, or at least about 75 years old. In some embodiments, the individual has one or more symptoms of chronic stress, including physical and psychological stress associated with the cancer, such as anxiety, depression, headache, pain, fatigue, insomnia, anorexia, nausea, malnutrition, or any combination thereof. In some embodiments, the individual has an advanced stage of cancer, such as any of T2, T3, T4, N1, N2, N3 or M1 stage of cancer based on the TNM staging system. In some embodiments, the individual has a high tumor burden, such as a large tumor size and/or a large number of cancer cells in the tumor bed. In some embodiments, the individual has palpable lymph nodes, or has cancer cells spread to nearby lymph nodes. In some embodiments, the individual has distant tumor metastases.

In some embodiments of any of the methods, the cancer is selected from the group consisting of lung cancer, uterine cancer, kidney cancer, ovarian cancer, breast cancer, endometrial cancer, head & neck cancer, pancreatic cancer, and melanoma. In some embodiments, the cancer is selected from the group consisting of breast cancer, lung cancer, and pancreatic cancer. In some embodiments, the cancer is triple negative breast cancer (TNBC). In some embodiments, the cancer is non-small cell lung cancer (NSCLC). In some embodiments, the cancer is pancreatic ductal adenocarcinoma (PDAC). In some embodiments, the cancer is selected from the group consisting of adrenocortical cancer, bile duct cancer, bladder cancer, breast cancer, cervical cancer, colon cancer, endometroid cancer, esophageal cancer, glioblasoma, head and neck cancer, kidney chromophobe cancer, kidney clear cell carcinoma, kidney papillary cell carcinoma, liver cancer, lower grade glioma, lung adenocarcinoma, lung squamous cell carcinoma, melanoma, mesothelioma, ocular melanomas, ovarian cancer, pancreatic cancer, pheochromocytoma and paraganglioma, prostate cancer, sarcoma, stomach cancer, testicular cancer, thyroid cancer, and uterine carcinosarcoma.

In some embodiments, the cancer is a solid epithelial tumor or a sarcoma. In some embodiments, the cancer is selected from a group consisting of adrenocortical carcinoma, Kaposi sarcoma, anal cancer, gastrointestinal carcinoid tumor, basal cell carcinoma, bile duct cancer, bladder cancer (such as bladder transitional cell carcinoma, bladder squamous cell carcinoma, and bladder adenocarcinoma), bone cancer (such as Ewing Sarcoma, osteosarcoma, chondrosarcoma, and malignant fibrous histiocytoma), breast cancer (such as ductal carcinoma, lobular carcinoma, fibroadenoma bronchial tumor, carcinoma of unknown primary, cervical cancer, chordoma, colon cancer, rectal cancer, endometrial cancer, esophageal cancer (including esophageal squamous cell carcinoma and esophageal adenocarcinoma), intraocular melanoma, ovarian cancer (such as ovarian epithelial cancer, Fallopian tube cancer, and peritoneal cancer), gallbladder cancer, gastric cancer, head and neck cancer (such as hypopharyngeal cancer, laryngeal cancer, lip and oral cavity cancer, metastatic squamous neck cancer with occult primary treatment, nasopharyngeal cancer, oropharyngeal cancer, paranasal sinus and nasal cavity cancer, salivary gland cancer, and oral complications of chemotherapy and head/neck radiation), heart tumor (such as rhabdomyoma, myxoma, fibroma, fibrosarcoma, and angiosarcoma), hepatocellular (liver) cancer, kidney cancer (such as renal cell cancer, transitional cell cancer of the renal pelvis and ureter, and Wilms tumor), lung cancer (such as non-small cell lung cancer, and small cell lung cancer), skin cancer (such as basal cell carcinoma, squamous cell carcinoma, neuroendocrine carcinoma of the skin, melanoma, and Merkel cell carcinoma), pancreatic cancer, pheochromocytoma, parathyroid cancer, penile cancer, pituitary tumor, prostate cancer, uterine sarcoma (such as leiomyosarcoma and endometrial stromal sarcoma), small intestine cancer (such as small intestine adenocarcinoma and small intestine sarcoma, and gastrointestinal stromal tumor), soft tissue sarcoma (such as adult soft tissue sarcoma, and childhood soft tissue sarcoma), thyroid cancer (such as papillary, follicular, medullary and anaplastic thyroid cancer), urethral cancer (including urethral transitional cell carcinoma, urethral squamous cell carcinoma, and urethral adenocarcinoma), vaginal cancer (such as vaginal squamous cell carcinoma and vaginal adenocarcinoma), and vulvar cancer.

In some embodiments of any of the methods, the method is first-line therapy.

In some embodiments, the cancer is at an advanced stage (such as stage III or stage IV). In some embodiments, the cancer is metastatic cancer.

In some embodiments of any of the methods, the cancer is pancreatic cancer. Pancreatic cancers that can be treated with methods described herein include, but are not limited to, exocrine pancreatic cancers and endocrine pancreatic cancers. Exocrine pancreatic cancers include, but are not limited to, adenocarcinomas, acinar cell carcinomas, adenosquamous carcinomas, colloid carcinomas, undifferentiated carcinomas with osteoclast-like giant cells, hepatoid carcinomas, intraductal papillary-mucinous neoplasms, mucinous cystic neoplasms, pancreatoblastomas, serous cystadenomas, signet ring cell carcinomas, solid and pseuodpapillary tumors, pancreatic ductal carcinomas, and undifferentiated carcinomas. In some embodiments, the exocrine pancreatic cancer is pancreatic ductal carcinoma. Endocrine pancreatic cancers include, but are not limited to, insulinomas and glucagonomas. In some embodiments, the pancreatic cancer is any of early stage pancreatic cancer, non-metastatic pancreatic cancer, primary pancreatic cancer, resected pancreatic cancer, advanced pancreatic cancer, locally advanced pancreatic cancer, metastatic pancreatic cancer, unresectable pancreatic cancer, pancreatic cancer in remission, recurrent pancreatic cancer, pancreatic cancer in an adjuvant setting, or pancreatic cancer in a neoadjuvant setting. In some embodiments, the pancreatic cancer is locally advanced pancreatic cancer, unresectable pancreatic cancer, or metastatic pancreatic ductal carcinoma. In some embodiments, the pancreatic cancer is resistant to the gemcitabine-based therapy. In some embodiments, the pancreatic cancer is refractory to the gemcitabine-based therapy.

In some embodiments, the individual has a pancreatic cancer (such as metastatic cancer). In some embodiments, the individual has locally advanced unresectable, pancreatic cancer. In some embodiments, the primary location of the pancreatic cancer is the head of the pancreas. In some embodiments, the primary location of the pancreatic cancer is the body of the pancreas. In some embodiments, the primary location of the pancreatic cancer is the tail of the pancreas. In some embodiments, the individual has metastasis in the liver. In some embodiments, the individual has pulmonary metastasis. In some embodiments, the individual has peritoneal carcinomatosis. In some embodiments, the individual has stage IV pancreatic cancer at the time of diagnosis of pancreatic cancer. In some embodiments, the individual has 3 or more metastatic sites. In some embodiments, the individual has more than 3 metastatic sites. In some embodiments, the individual has a serum CA19-9 level that is ≧59×ULN (Upper Limit of Normal). In some embodiments, the individual has Karnofsky performance status (KPS) of between 70 and 80. In some embodiments, the individual has adenocarcinoma of the pancreas.

The methods described herein for treating pancreatic cancer can be used in monotherapy as well as in combination therapy with another agent. In some embodiments, the other agent is gemcitabine. In some embodiments, the other agent is an agent that inhibits GR expression or activity as further discussed in the sections below.

In some embodiments, there is provided a method of treating locally advanced unresectable or metastatic adenocarcinoma of the pancreas in a human individual comprising intravenously administering (such as by intravenous infusion over about 30 to about 40 minutes) to the individual (i) an effective amount of Nab-paclitaxel (for example about 5 mg/ml Nab-paclitaxel); and (ii) an effective amount of gemcitabine, wherein the dose of paclitaxel in the nanoparticle composition is about 125 mg/m² on days 1, 8, and 15 of each 28-day cycle, wherein the dose of gemcitabine is about 1000 mg/m² on days 1, 8, and 15 of each 28-day cycle, wherein the individual has a high level of a GR. In some embodiments, the gemcitabine is administered immediately after the completion of the administration of the nanoparticle composition. In some embodiments, the individual is characterized by a high level of GR expression. In some embodiments, the individual is characterized by a high level of GR activity. In some embodiments, the individual is characterized by a high level of GR expression and a high level of GR activity. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined (e.g., high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein).

In some embodiments, there is provided a method of treating locally advanced unresectable or metastatic adenocarcinoma of the pancreas in a human individual comprising intravenously administering (such as by intravenous infusion over about 30 to about 40 minutes) to the individual (i) an effective amount of Nab-paclitaxel (for example about 5 mg/ml Nab-paclitaxel); and (ii) an effective amount of gemcitabine, wherein the dose of paclitaxel in the nanoparticle composition is about 125 mg/m²on days 1, 8, and 15 of each 28-day cycle, wherein the dose of gemcitabine is about 1000 mg/m² on days 1, 8, and 15 of each 28-day cycle, wherein the individual is selected for treatment based on a high level of GR. In some embodiments, the gemcitabine is administered immediately after the completion of the administration of the nanoparticle composition.

In some embodiments, there is provided a method of treating locally advanced unresectable or metastatic adenocarcinoma of the pancreas in a human individual comprising intravenously administering (such as by intravenous infusion over about 30 to about 40 minutes) to the individual (i) an effective amount of Nab-paclitaxel (for example about 5 mg/ml Nab-paclitaxel); and (ii) an effective amount of gemcitabine, wherein the dose of paclitaxel in the nanoparticle composition is about 125 mg/m2 on days 1, 8, and 15 of each 28-day cycle, wherein the dose of gemcitabine is about 1000 mg/m2 on days 1, 8, and 15 of each 28-day cycle, wherein the individual has a high level of a GC (such as cortisol). In some embodiments, the gemcitabine is administered immediately after the completion of the administration of the nanoparticle composition. In some embodiments, the individual is characterized by a high level of GC (such as cortisol) secretion. In some embodiments, the individual is characterized by a high level of GC (such as cortisol) activity. In some embodiments, the individual is characterized by a high level of GC (such as cortisol) secretion and a high level of GC (such as cortisol) activity. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein).

In some embodiments, there is provided a method of treating locally advanced unresectable or metastatic adenocarcinoma of the pancreas in a human individual comprising intravenously administering (such as by intravenous infusion over about 30 to about 40 minutes) to the individual (i) an effective amount of Nab-paclitaxel (for example about 5 mg/ml Nab-paclitaxel); and (ii) an effective amount of gemcitabine, wherein the dose of paclitaxel in the nanoparticle composition is about 125 mg/m² on days 1, 8, and 15 of each 28-day cycle, wherein the dose of gemcitabine is about 1000 mg/m2 on days 1, 8, and 15 of each 28-day cycle, wherein the individual is selected for treatment based on a high level of GC (such as cortisol). In some embodiments, the gemcitabine is administered immediately after the completion of the administration of the nanoparticle composition.

In some embodiments, there is provided a method of treating locally advanced unresectable or metastatic adenocarcinoma of the pancreas in a human individual comprising intravenously administering (such as by intravenous infusion over about 30 to about 40 minutes) to the individual (i) an effective amount of Nab-paclitaxel (for example about 5 mg/ml Nab-paclitaxel); and (ii) an effective amount of gemcitabine, wherein the dose of paclitaxel in the nanoparticle composition is about 125 mg/m2 on days 1, 8, and 15 of each 28-day cycle, wherein the dose of gemcitabine is about 1000 mg/m2 on days 1, 8, and 15 of each 28-day cycle, wherein the individual has a high level of a GR and GC (such as cortisol). In some embodiments, the gemcitabine is administered immediately after the completion of the administration of the nanoparticle composition.

In some embodiments, there is provided a method of treating locally advanced unresectable or metastatic adenocarcinoma of the pancreas in a human individual comprising intravenously administering (such as by intravenous infusion over about 30 to about 40 minutes) to the individual (i) an effective amount of Nab-paclitaxel (for example about 5 mg/ml Nab-paclitaxel); and (ii) an effective amount of gemcitabine, wherein the dose of paclitaxel in the nanoparticle composition is about 125 mg/m2 on days 1, 8, and 15 of each 28-day cycle, wherein the dose of gemcitabine is about 1000 mg/m2 on days 1, 8, and 15 of each 28-day cycle, wherein the individual is selected for treatment based on a high level of GR and GC (such as cortisol). In some embodiments, the gemcitabine is administered immediately after the completion of the administration of the nanoparticle composition.

In some embodiments of any of the methods, the cancer is breast cancer. Breast cancers that can be treated with methods described herein include any of stage 0, stage 1, stage II, stage III, or stage IV breast cancer according to the staging criteria included in AJCC Cancer Staging Manual, 6th Edition, 2002. In some embodiments, the breast cancer is inflammatory breast cancer. In some embodiments, the cancer is a primary breast tumor. In some embodiments, the breast cancer is locally advanced breast cancer. In some embodiments, the breast cancer is recurrent breast cancer. In some embodiments, the breast cancer has reoccurred after remission. In some embodiments, the breast cancer is progressive breast cancer. In some embodiments, the breast cancer is breast cancer in remission. In some embodiments, the breast cancer is not metastatic. In some embodiments of any of the above methods, the breast cancer is metastatic. In some embodiments, the individual has distant metastases. In some embodiments, the individual does not have distant metastases. In some embodiments, the breast cancer is substantially refractory to hormone therapy. In some embodiments, the breast cancer is localized resectable, localized unresectable, or unresectable.

In some embodiments, the breast cancer is breast cancer in an adjuvant setting, ductal carcinoma in situ (DCIS), invasive ductal carcinoma (IDC), or breast cancer in a neoadjuvant setting. In some embodiments, the breast cancer is hormone receptor positive metastatic breast cancer. In some embodiments, the breast cancer is negative for at least one of estrogen receptor (“ER”), progesterone receptor (“PR”) or human epidermal growth factor receptor 2 (“HER2”). In some embodiments, the breast cancer is Triple Negative Breast Cancer (TNBC) (i.e. ER-negative, PR-negative and HER2-negative). In some embodiments, the breast cancer (which may be HER2 positive or HER2 negative) is advanced breast cancer. In some embodiments, the individual may be a human who has a gene, genetic mutation, or polymorphism associated with breast cancer (e.g., BRCA1, BRCA2, ATM, CHEK2, RAD51, AR, DIRAS3, ERBB2, TP53, AKT, PTEN, and/or PI3K) or has one or more extra copies of a gene (e.g., one or more extra copies of the HER2 gene) associated with breast cancer.

In some embodiments, there is provided a method of treating metastatic breast cancer in a human individual comprising intravenously administering (such as by intravenous infusion over about 30 minutes) to the individual an effective amount of Nab-paclitaxel (for example about 5 mg/ml Nab-paclitaxel), wherein the dose of paclitaxel in the nanoparticle composition is about 260 mg/m² every 3 weeks, wherein the individual has a high level of a GR. In some embodiments, the individual has previously received combination chemotherapy for metastatic disease, or has relapsed within 6 months of adjuvant chemotherapy. In some embodiments, the individual has received an antracycline in previous therapy. In some embodiments, the individual is characterized by a high level of GR expression. In some embodiments, the individual is characterized by a high level of GR activity. In some embodiments, the individual is characterized by a high level of GR expression and a high level of GR activity. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein).

In some embodiments, there is provided a method of treating metastatic breast cancer in a human individual comprising intravenously administering (such as by intravenous infusion over about 30 minutes) to the individual an effective amount of Nab-paclitaxel (for example about 5 mg/ml Nab-paclitaxel), wherein the dose of paclitaxel in the nanoparticle composition is about 260 mg/m² every 3 weeks, wherein the individual is selected for treatment based on a high level of GR. In some embodiments, the individual has previously received combination chemotherapy for metastatic disease, or has relapsed within 6 months of adjuvant chemotherapy. In some embodiments, the individual has received an antracycline in previous therapy.

In some embodiments, there is provided a method of treating metastatic breast cancer in a human individual comprising intravenously administering (such as by intravenous infusion over about 30 minutes) to the individual an effective amount of Nab-paclitaxel (for example about 5 mg/ml, Nab-paclitaxel), wherein the dose of paclitaxel in the nanoparticle composition is about 260 mg/m² every 3 weeks, wherein the individual has a high level of a GC (such as cortisol). In some embodiments, the individual has previously received combination chemotherapy for metastatic disease, or has relapsed within 6 months of adjuvant chemotherapy. In some embodiments, the individual has received an antracycline in previous therapy. In some embodiments, the individual is characterized by a high level of GC (such as cortisol) secretion. In some embodiments, the individual is characterized by a high level of GC (such as cortisol) activity. In some embodiments, the individual is characterized by a high level of GC (such as cortisol) secretion and a high level of GC (such as cortisol) activity. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein).

In some embodiments, there is provided a method of treating metastatic breast cancer in a human individual comprising intravenously administering (such as by intravenous infusion over about 30 minutes) to the individual an effective amount of Nab-paclitaxel (for example about 5 mg/ml Nab-paclitaxel), wherein the dose of paclitaxel in the nanoparticle composition is about 260 mg/m² every 3 weeks, wherein the individual is selected for treatment based on a high level of GC (such as cortisol). In some embodiments, the individual has previously received combination chemotherapy for metastatic disease, or has relapsed within 6 months of adjuvant chemotherapy. In some embodiments, the individual has received an antracycline in previous therapy.

In some embodiments, there is provided a method of treating metastatic breast cancer in a human individual comprising intravenously administering (such as by intravenous infusion over about 30 minutes) to the individual an effective amount of Nab-paclitaxel (for example about 5 mg/ml Nab-paclitaxel), wherein the dose of paclitaxel in the nanoparticle composition is about 260 mg/m² every 3 weeks, wherein the individual has a high level of a GR and GC (such as cortisol). In some embodiments, the individual has previously received combination chemotherapy for metastatic disease, or has relapsed, within 6 months of adjuvant chemotherapy. In some embodiments, the individual has received an antracycline in previous therapy.

In some embodiments, there is provided a method of treating metastatic breast cancer in a human individual comprising intravenously administering (such as by intravenous infusion over about 30 minutes) to the individual an effective amount of Nab-paclitaxel (for example about 5 mg/ml Nab-paclitaxel), wherein the dose of paclitaxel in the nanoparticle composition is about 260 mg/m² every 3 weeks, wherein the individual is selected for treatment based on a high level of GR and GC (such as cortisol). In some embodiments, the individual has previously received, combination chemotherapy for metastatic disease, or has relapsed within 6 months of adjuvant chemotherapy. In some embodiments, the individual has received an antracycline in previous therapy.

In some embodiments of any of the methods, the cancer is lung cancer. In some embodiments of any of the methods, the cancer is non-small cell lung cancer (NSCLC). NSCLC that can be treated with methods described herein include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma (e.g., acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the cancer is small cell lung cancer (SCLC; also called oat cell carcinoma). The small cell lung cancer may be limited-stage, extensive stage or recurrent small cell lung cancer.

In some embodiments, the NSCLC is, according to TNM classifications, a stage T tumor (primary tumor), a stage N tumor (regional lymph nodes), or a stage M tumor (distant metastasis). In some embodiments, the NSCLS is an occult tumor, a stage 0 tumor, a stage I tumor (stage IA (T1, NO, M0) or stage IB (T2, NO, M0)), a stage II tumor (stage HA (T1, N1, M0) and stage IB (T2, N1, M0)), a stage IIIA tumor (T1, N2, M0, T2, N2, M0, T3, N1, M0, or T3, N2, M0), a stage IIIB tumor (Any T, N3, M0 or T4, any N, M0), or a stage IV tumor (Any T, any N, M1). In some embodiments, the NSCLC is early stage NSCLC, non-metastatic NSCLC, primary NSCLC, advanced NSCLC, locally advanced NSCLC, metastatic NSCLC, NSCLC in remission, or recurrent NSCLC. In some embodiments, the NSCLC is localized resectable, localized unresectable, or unresectable. In some embodiments, the NSCLC is unresectable stage IV NSCLC. In some embodiments, the NSCLC is inoperable Stage IIIA and/or IIIB NSCLC, PS 0-1, and FEY 1>800 ml.

The methods described herein for treating NSCLC can be used in monotherapy as well as in combination therapy with another agent. In some embodiments, the other agent is carboplatin. In some embodiments, the other agent is an agent that inhibits GR expression or activity as further discussed in the sections below.

In some embodiments, there is provided a method of treating locally advanced unresectable or metastatic non-small cell lung cancer (NSCLC) in a human individual comprising intravenously administering (such as by intravenous infusion over about 30 minutes) to the individual (i) an effective amount of Nab-paclitaxel (for example about 5 mg/ml Nab-paclitaxel); and (ii) an effective amount of carboplatin, wherein the dose of paclitaxel in the nanoparticle composition is about 100 mg/m² on days 1, 8, and 15 of each 21-day cycle, wherein the dose of carboplatin is about AUC=6 mg·min/mL on day 1 of each 21-day cycle, wherein the individual has a high level of a GR. In some embodiments, the carboplatin is administered immediately after the completion of the administration of the nanoparticle composition. In some embodiments, the individual is characterized by a high level of GR expression. In some embodiments, the individual is characterized by a high level of GR activity. In some embodiments, the individual is characterized by a high level of GR expression and a high level of GR activity. In some embodiments, the level of GR expression is based on protein expression. In some embodiments, the level of GR expression is based on mRNA level. In some embodiments, the level of GR activity is determined by measuring the expression or activity of a GR responsive molecule. In some embodiments, the level is determined (e.g., high or low) by comparing to a control (such as any of the controls described herein). In some embodiments, the method further comprises comparing the level of the GR with a control. In some embodiments, the level is determined (e.g., high or low) based on a scoring system (such as any of the scoring systems described herein).

In some embodiments, there is provided a method of treating locally advanced unresectable or metastatic non-small cell lung cancer (NSCLC) in a human individual comprising intravenously administering (such as by intravenous infusion over about 30 minutes) to the individual (i) an effective amount of Nab-paclitaxel (for example about 5 mg/ml Nab-paclitaxel); and (ii) an effective amount of carboplatin, wherein the dose of paclitaxel in the nanoparticle composition is about 100 mg/m² on days 1, 8, and 15 of each 21-day cycle, wherein the dose of carboplatin is about AUC=6 mg·min/mL on day 1 of each 21-day cycle, wherein the individual is selected for treatment based on a high level of GR. In some embodiments, the carboplatin is administered immediately after the completion of the administration of the nanoparticle composition.

In some embodiments, there is provided a method of treating locally advanced unresectable or metastatic non-small cell lung cancer (NSCLC) in a human individual comprising intravenously administering (such as by intravenous infusion over about 30 minutes) to the individual (i) an effective amount of Nab-paclitaxel (for example about 5 mg/ml Nab-paclitaxel); and (ii) an effective amount of carboplatin, wherein the dose of paclitaxel in the nanoparticle composition is about 100 mg/m² on days 1, 8, and 15 of each 21-day cycle, wherein the dose of carboplatin is about AUC-6 mg·min/mL on day 1 of each 21-day cycle, wherein the individual has a high level of a GC (such as cortisol). In some embodiments, the carboplatin is administered immediately after the completion of the administration of the nanoparticle composition. In some embodiments, the individual is characterized by a high level of GC (such as cortisol) secretion. In some embodiments, the individual is characterized by a high level of GC (such as cortisol) activity. In some embodiments, the individual is characterized by a high level of GC (such as cortisol) secretion and a high level of GC (such as cortisol) activity. In some embodiments, the level of GC secretion is based on endogenous GC secretion (such as cortisol secretion). In some embodiments, the level of GC activity is based on endogenous GC activity (such as cortisol activity). In some embodiments, the level of GC (such as cortisol) secretion is based on free GC (such as cortisol) in the body (such as blood, urine and saliva). In some embodiments, the level is determined (e.g., high or low) by comparing to a control (such as any of the controls described herein). In some embodiments, the method further comprises comparing the level of the GC (such as cortisol) with a control. In some embodiments, the level is determined (e.g., high or low) based on a scoring system (such as any of the scoring systems described herein).

In some embodiments, there is provided a method of treating locally advanced unresectable or metastatic non-small cell lung cancer (NSCLC) in a human individual comprising intravenously administering (such as by intravenous infusion over about 30 minutes) to the individual (i) an effective amount of Nab-paclitaxel (for example about 5 mg/ml Nab-paclitaxel); and (ii) an effective amount of carboplatin, wherein the dose of paclitaxel in the nanoparticle composition is about 100 mg/m² on days 1, 8, and 15 of each 21-day cycle, wherein the dose of carboplatin is about AUC=6 mg·min/mL on day 1 of each 21-day cycle, wherein the individual is selected for treatment based on a high level of GC (such as cortisol). In some embodiments, the carboplatin is administered immediately after the completion of the administration of the nanoparticle composition.

In some embodiments, there is provided a method of treating locally advanced unresectable or metastatic non-small cell lung cancer (NSCLC) in a human individual comprising intravenously administering (such as by intravenous infusion over about 30 minutes) to the individual (i) an effective amount of Nab-paclitaxel (for example about 5 mg/ml Nab-paclitaxel); and (ii) an effective amount of carboplatin, wherein the dose of paclitaxel in the nanoparticle composition is about 100 mg/m² on days 1, 8, and 15 of each 21-day cycle, wherein the dose of carboplatin is about AUC-6 mg·min/mL on day 1 of each 21-day cycle, wherein the individual has a high level of a GR and GC (such as cortisol). In some embodiments, the carboplatin is administered immediately after the completion of the administration of the nanoparticle composition.

In some embodiments, there is provided a method of treating locally advanced unresectable or metastatic non-small cell lung cancer (NSCLC) in a human individual comprising intravenously administering (such as by intravenous infusion over about 30 minutes) to the individual (i) an effective amount of Nab-paclitaxel (for example about 5 mg/ml Nab-paclitaxel); and (ii) an effective amount of carboplatin, wherein the dose of paclitaxel in the nanoparticle composition is about 100 mg/m² on days 1, 8, and 15 of each 21-day cycle, wherein the dose of carboplatin is about AUC=6 mg·min/mL on day 1 of each 21-day cycle, wherein the individual is selected for treatment based on a high level of GR and GC (such as cortisol). In some embodiments, the carboplatin is administered immediately after the completion of the administration of the nanoparticle composition.

The methods described herein can be used for any one or more of the following purposes: alleviating one or more symptoms of cancer, delaying progression of cancer, shrinking cancer tumor size, disrupting (such as destroying) cancer stroma, inhibiting cancer tumor growth, prolonging overall survival, prolonging disease-free survival, prolonging time to cancer disease progression, preventing or delaying cancer tumor metastasis, reducing (such as eradiating) preexisting cancer tumor metastasis, reducing incidence or burden of preexisting cancer tumor metastasis, preventing recurrence of cancer, and/or improving clinical benefit of cancer.

The methods described herein for treating cancer can be used in monotherapy as well as in combination therapy with another agent. In some embodiments, the other agent is an agent that down-regulates GR, for example by inhibiting GR expression or activity as further discussed in the sections below.

### Methods of Combination Therapy

In another aspect, the present application provides methods of treating an individual having a cancer, comprising administering to the individual: a) an effective amount of a composition comprising a taxane (such as a composition comprising nanoparticles comprising a taxane and an albumin); and b) an effective amount of a GR down-regulator. The combination therapy method described herein can be used independent of or in conjunction with the methods described above based on GR and/or GC levels.

Thus, in some embodiments, there is provided a method of treating an individual having a cancer, comprising administering to the individual: a) an effective amount of a composition comprising a taxane (for example a cremophor-free formulation of taxane); and b) an effective amount of another agent that down-regulates GR. In some embodiments, there is provided a method of treating an individual having a cancer, comprising administering to the individual: a) an effective amount of a composition comprising nanoparticles comprising a taxane (such as paclitaxel); and b) an effective amount of another agent that down-regulates GR. In some embodiments, there is provided a method of treating an individual having a cancer, comprising administering to the individual: a) an effective amount of a composition comprising nanoparticles comprising a taxane (such as paclitaxel) and an albumin; and b) an effective amount of another agent that down-regulates GR. In some embodiments, there is provided a method of treating an individual having a cancer, comprising administering to the individual: a) an effective amount of a composition comprising nanoparticles comprising paclitaxel coated with albumin (including nanoparticles having an average diameter of no greater than about 200 nm); and b) an effective amount of another agent that down-regulates GR. In some embodiments, there is provided a method of treating an individual having a cancer, comprising administering to the individual: a) an effective amount of Nab-paclitaxel (for example about 5 mg/ml Nab-paclitaxel); and b) an effective amount of another agent that down-regulates GR. In some embodiments, the cancer is pancreatic cancer. In some embodiments, the method does not require premedication. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments, there is provided a method of treating an individual having a cancer, comprising administering to the individual: a) an effective amount of a composition comprising a taxane (for example a cremophor-free formulation of taxane); and b) an effective amount of another agent that inhibits GR expression (for example an RNAi or antisense RNA specific for GR). In some embodiments, there is provided a method of treating an individual having a cancer, comprising administering to the individual: a) an effective amount of a composition comprising nanoparticles comprising a taxane (such as paclitaxel); and b) an effective amount of another agent that inhibits GR expression (for example an RNAi or antisense RNA specific for GR). In some embodiments, there is provided a method of treating an individual having a cancer, comprising administering to the individual: a) an effective amount of a composition comprising nanoparticles comprising a taxane (such as paclitaxel) and an albumin; and b) an effective amount of another agent that inhibits GR expression (for example an RNAi or antisense RNA specific for GR). In some embodiments, there is provided a method of treating an individual having a cancer, comprising administering to the individual: a) an effective amount of a composition comprising nanoparticles comprising paclitaxel coated with albumin (including nanoparticles having an average diameter of no greater than about 200 nm); and b) an effective amount of another agent that inhibits GR expression (for example an RNAi or antisense RNA specific for GR). In some embodiments, there is provided a method of treating an individual having a cancer, comprising administering to the individual: a) an effective amount of Nab-paclitaxel (for example about 5 mg/ml Nab-paclitaxel); and b) an effective amount of another agent that inhibits GR expression (for example an RNAi or antisense RNA specific for GR). In some embodiments, the cancer is pancreatic cancer. In some embodiments, the method does not require premedication. In some embodiments, the taxane composition and the other agent are administered sequentially. In some embodiments, the taxane composition and the other agent are administered simultaneously. In some embodiments, the individual is characterized by high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments, there is provided a method of treating an individual having a cancer, comprising administering to the individual: a) an effective amount of a composition comprising a taxane (for example a cremophor-free formulation of taxane); and b) an effective amount of another agent that inhibits GR activity. In some embodiments, there is provided a method of treating an individual having a cancer, comprising administering to the individual: a) an effective amount of a composition comprising nanoparticles comprising a taxane (such as paclitaxel); and b) an effective amount of another agent that inhibits GR activity. In some embodiments, there is provided a method of treating an individual having a cancer, comprising administering to the individual: a) an effective amount of a composition comprising nanoparticles comprising a taxane (such as paclitaxel) and an albumin; and b) an effective amount of another agent that inhibits GR activity. In some embodiments, there is provided a method of treating an individual having a cancer, comprising administering to the individual: a) an effective amount of a composition comprising nanoparticles comprising paclitaxel coated with albumin (including nanoparticles having an average diameter of no greater than about 200 nm); and b) an effective amount of another agent that inhibits GR activity. In some embodiments, there is provided a method of treating an individual having a cancer, comprising administering to the individual: a) an effective amount of Nab-paclitaxel (for example about 5 mg/ml Nab-paclitaxel); and b) an effective amount of another agent that inhibits GR activity. In some embodiments, the cancer is pancreatic cancer. In some embodiments, the method does not require premedication. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments, there is provided a method of treating an individual having a cancer, comprising administering to the individual: a) an effective amount of a composition comprising a taxane (for example a cremophor-free formulation of taxane); and b) an effective amount of another agent, wherein the other agent is a GR antagonist (such as mifepristone). In some embodiments, there is provided a method of treating an individual having a cancer, comprising administering to the individual: a) an effective amount of a composition comprising nanoparticles comprising a taxane (such as paclitaxel); and b) an effective amount of another agent, wherein the other agent is a GR antagonist (such as mifepristone). In some embodiments, there is provided a method of treating an individual having a cancer, comprising administering to the individual: a) an effective amount of a composition comprising nanoparticles comprising a taxane (such as paclitaxel) and an albumin; and b) an effective amount of another agent, wherein the other agent is a GR antagonist (such as mifepristone). In some embodiments, there is provided a method of treating an individual having a cancer, comprising administering to the individual: a) an effective amount of a composition comprising nanoparticles comprising paclitaxel coated with albumin (including nanoparticles having an average diameter of no greater than about 200 nm); and b) an effective amount of another agent, wherein the other agent is a GR antagonist (such as mifepristone). In some embodiments, there is provided a method of treating an individual having a cancer, comprising administering to the individual: a) an effective amount of Nab-paclitaxel (for example about 5 mg/ml Nab-paclitaxel); and b) an effective amount of another agent, wherein the other agent is a GR antagonist (such as mifepristone). In some embodiments, the cancer is pancreatic cancer. In some embodiments, the method does not require premedication. In some embodiments, the taxane composition and the GR antagonist (such as mifepristone) are administered sequentially. In some embodiments, the taxane composition and the GR antagonist (such as mifepristone) are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments, there is provided a method of treating an individual having a cancer, comprising administering to the individual: a) an effective amount of a composition comprising a taxane (for example a cremophor-free formulation of taxane); and b) an effective amount of another agent, wherein the other agent is a modulator of a GR responsive molecule. In some embodiments, there is provided a method of treating an individual having a cancer, comprising administering to the individual: a) an effective amount of a composition comprising nanoparticles comprising a taxane (such as paclitaxel); and b) an effective amount of another agent, wherein the other agent is a modulator of a GR responsive molecule. In some embodiments, there is provided a method of treating an individual having a cancer, comprising administering to the individual: a) an effective amount of a composition comprising nanoparticles comprising a taxane (such as paclitaxel) and an albumin; and b) an effective amount of another agent, wherein the other agent is a modulator of a GR responsive molecule. In some embodiments, there is provided a method of treating an individual having a cancer, comprising administering to the individual: a) an effective amount of a composition comprising nanoparticles comprising paclitaxel coated with albumin (including nanoparticles having an average diameter of no greater than about 200 nm); and b) an effective amount of another agent, wherein the other agent is a modulator of a GR responsive molecule. In some embodiments, there is provided a method of treating an individual having a cancer, comprising administering to the individual: a) an effective amount of Nab-paclitaxel (for example about 5 mg/ml Nab-paclitaxel); and b) an effective amount of another agent, wherein the other agent is a modulator of a GR responsive molecule. In some embodiments, the cancer is pancreatic cancer. In some embodiments, the method does not require premedication. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments, there is provided a method of treating an individual having a cancer, comprising administering to the individual: a) an effective amount of a composition comprising a taxane (for example a cremophor-free formulation of taxane); and b) an effective amount of another agent, wherein the other agent is a modulator of a molecule selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FAM134A, NRIP1, RAC2, SPP1, PHF15, BTN3A2, SESN1, MAP3K5, DPYSL2, SEMA4D, STOM, MAOA, SLUG,SERPINE1, RGS2, KRT7, MME, JAK2, CEBPD, IL6, LIF, and TNFRSF11B. In some embodiments, there is provided a method of treating an individual having a cancer, comprising administering to the individual: a) an effective amount of a composition comprising nanoparticles comprising a taxane (such as paclitaxel); and b) an effective amount of another agent, wherein the other agent is a modulator of a molecule selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG,SERPINE1, RGS2, KRT7, MME, JAK2, CEBPD, IL6, LIF, and TNFRSF11B. In some embodiments, there is provided a method of treating an individual having a cancer, comprising administering to the individual; a) an effective amount of a composition comprising nanoparticles comprising a taxane (such as paclitaxel) and an albumin; and b) an effective amount of another agent, wherein the other agent is a modulator of a molecule selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG,SERPINE1, RGS2, KRT7, MME, JAK2, CEBPD, IL6, LIF, and TNFRSF11B. In some embodiments, there is provided a method of treating an individual having a cancer, comprising administering to the individual; a) an effective amount of a composition comprising nanoparticles comprising paclitaxel coated with albumin (including nanoparticles having an average diameter of no greater than about 200 nm); and b) an effective amount of another agent, wherein the other agent is a modulator of a molecule selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG,SERPINE1, RGS2, KRT7, MME, JAK2, CEBPD, IL6, LIF, and TNFRSF11B. In some embodiments, there is provided a method of treating an individual having a cancer, comprising administering to the individual: a) an effective amount of Nab-paclitaxel (for example about 5 mg/ml Nab-paclitaxel); and b) an effective amount of another agent, wherein the other agent is a modulator of a molecule selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG,SERPINE1, RGS2, KRT7, MME, JAK2, CEBPD, IL6, LIF, and TNFRSF11B. In some embodiments, the cancer is pancreatic cancer. In some embodiments, the method does not require premedication. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments, there is provided a method of treating an individual having a cancer, comprising administering to the individual: a) an effective amount of a composition comprising a taxane (for example a cremophor-free formulation of taxane); and b) an effective amount of another agent, wherein the other agent is a modulator of a molecule selected from the group consisting of SGK1, MKP1, MCL-1, Bcl2, BCLxL, AKT, MAP Tau, INK1, c-Jun and AP-1. In some embodiments, there is provided a method of treating an individual having a cancer, comprising administering to the individual: a) an effective amount of a composition comprising nanoparticles comprising a taxane (such as paclitaxel); and b) an effective amount of another agent, wherein the other agent is a modulator of a molecule selected from the group consisting of SGK1, MKP1, MCL-1, Bcl2, BCLxL, AKT, MAP Tau, JNK1, c-Jun and AP-1. In some embodiments, there is provided a method of treating an individual having a cancer, comprising administering to the individual: a) an effective amount of a composition comprising nanoparticles comprising a taxane (such as paclitaxel) and an albumin; and b) an effective amount of another agent, wherein the other agent is a modulator of a molecule selected from the group consisting of SGK1, MKP1, MCL-1, Bcl2, BCLxL, AKT, MAP Tau, INK1, c-Jun and AP-1. In some embodiments, there is provided a method of treating an individual having a cancer, comprising administering to the individual: a) an effective amount of a composition comprising nanoparticles comprising paclitaxel coated with albumin (including nanoparticles having an average diameter of no greater than about 200 nm); and b) an effective amount of another agent, wherein the other agent is a modulator of a molecule selected from the group consisting of SGK1, MKP1, MCL-1, Bcl2, BCLxL, AKT, MAP Tau, JNK1, c-Jun and AP-1. In some embodiments, there is provided a method of treating an individual having a cancer, comprising administering to the individual: a) an effective amount of Nab-paclitaxel (for example about 5 mg/ml Nab-paclitaxel); and b) an effective amount of another agent, wherein the other agent is a modulator of a molecule selected from the group consisting of SGK1, MKP1, MCL-1, Bcl2, BCLxL, AKT, MAP Tau, JNK1 c-Jun and AP-1. In some embodiments, the cancer is pancreatic cancer. In some embodiments, the method does not require premedication. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments, there is provided a method of treating an individual having a cancer, comprising administering to the individual: a) an effective amount of a composition comprising a taxane (for example a cremophor-free formulation of taxane); and b) an effective amount of another agent, wherein the other agent is a modulator of a molecule selected from the group consisting of FN1, SERPINE1, SLUG, RGS2, KRT7, MME, IL1R1, JAK2, CEBPD, MCL1, IL6, LIF, and TNFRSF11B. In some embodiments, there is provided a method of treating an individual having a cancer, comprising administering to the individual: a) an effective amount of a composition comprising nanoparticles comprising a taxane (such as paclitaxel); and b) an effective amount of another agent, wherein the other agent is a modulator of a molecule selected from the group consisting of FN1, SERPINE1, SLUG, RGS2, KRT7, MME, IL1R1, JAK2, CEBPD, MCL1, IL6, LIF, and TNFRSF11B. In some embodiments, there is provided a method of treating an individual having a cancer, comprising administering to the individual: a) an effective amount of a composition comprising nanoparticles comprising a taxane (such as paclitaxel) and an albumin; and b) an effective amount of another agent, wherein the other agent is a modulator of a molecule selected from the group consisting of FN1, SERPINE1, SLUG, RGS2, KRT7, MME, IL1R1, JAK2, CEBPD, MCL1, IL6, LIF, and TNFRSF11B. In some embodiments, there is provided a method of treating an individual having a cancer, comprising administering to the individual: a) an effective amount of a composition comprising nanoparticles comprising paclitaxel coated with albumin (including nanoparticles having an average diameter of no greater than about 200 nm); and b) an effective amount of another agent, wherein the other agent is a modulator of a molecule selected from the group consisting of FN1, SERPINE1, SLUG, RGS2, KRT7, MME, IL1R1, JAK2, CEBPD, MCL1, IL6, LIF, and TNFRSF11B. In some embodiments, there is provided a method of treating an individual having a cancer, comprising administering to the individual; a) an effective amount of Nab-paclitaxel (for example about 5 mg/ml Nab-paclitaxel); and b) an effective amount of another agent, wherein the other agent is a modulator of a molecule selected from the group consisting of FN1, SERPINE1, SLUG, RGS2, KRT7, MME, IL1R1, JAK2, CEBPD, MCL1, IL6, LIF, and TNFRSF11B. In some embodiments, the cancer is pancreatic cancer. In some embodiments, the method does not require premedication. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments, there is provided a method of treating an individual having a cancer, comprising administering to the individual: a) an effective amount of a composition comprising a taxane (for example a cremophor-free formulation of taxane); and b) an effective amount of another agent, wherein the other agent inhibits the epithelial-to-mesenchymal transition (EMT) pathway (such as an inhibitor of SLUG, for example, an RNAi agent against SLUG). In some embodiments, there is provided a method of treating an individual having a cancer, comprising administering to the individual; a) an effective amount of a composition comprising nanoparticles comprising a taxane (such as paclitaxel); and b) an effective amount of another agent, wherein the other agent inhibits the epithelial-to-mesenchymal transition (EMT) pathway (such as an inhibitor of SLUG, for example, an RNAi agent against SLUG). In some embodiments, there is provided a method of treating an individual having a cancer, comprising administering to the individual: a) an effective amount of a composition comprising nanoparticles comprising a taxane (such as paclitaxel) and an albumin; and b) an effective amount of another agent, wherein the other agent inhibits the epithelial-to-mesenchymal transition (EMT) pathway (such as an inhibitor of SLUG, for example, an RNAi agent against SLUG). In some embodiments, there is provided a method of treating an individual having a cancer, comprising administering to the individual: a) an effective amount of a composition comprising nanoparticles comprising paclitaxel coated with albumin (including nanoparticles having an average diameter of no greater than about 200 nm); and b) an effective amount of another agent, wherein the other agent inhibits the epithelial-to-mesenchymal transition (EMT) pathway (such as an inhibitor of SLUG, for example, an RNAi agent against SLUG). In some embodiments, there is provided a method of treating an individual having a cancer, comprising administering to the individual: a) an effective amount of Nab-paclitaxel (for example about 5 mg/ml Nab-paclitaxel); and b) an effective amount of another agent, wherein the other agent inhibits the epithelial-to-mesenchymal transition (EMT) pathway (such as an inhibitor of SLUG, for example, an RNAi agent against SLUG). In some embodiments, the cancer is pancreatic cancer. In some embodiments, the method does not require premedication. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments of any of the methods, the method comprises a method of inhibiting cancer cell proliferation (such as tumor growth) in an individual, comprising administering to the individual a) an effective amount of a composition comprising a taxane (such as a composition comprising nanoparticles comprising a taxane (e.g., paclitaxel) and an albumin) and b) an effective amount of another agent that down-regulates GR (such as inhibits GR expression or activity). In some embodiments, at least about 10% (including for example at least about any of 20%, 30%, 40%, 60%, 70%, 80%, 90%, or 100%) cell proliferation is inhibited. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments of any of the methods, the method comprises a method of promoting apoptosis of cancer cells in an individual, comprising administering to the individual a) an effective amount of a composition comprising a taxane (such as a composition comprising nanoparticles comprising a taxane (e.g., paclitaxel) and an albumin) and b) an effective amount of another agent that down-regulates GR (such as inhibits GR expression or activity). In some embodiments, apoptosis of cancer cells is increased by at least about 10% (including for example at least about any of 20%, 50%, 1 fold, 2 fold, 3 fold, 5 fold, 10 fold, or more). In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments of any of the methods, the method comprises a method of inhibiting tumor metastasis in an individual, comprising administering to the individual a) an effective amount of a composition comprising a taxane (such as a composition comprising nanoparticles comprising a taxane (e.g., paclitaxel) and an albumin) and b) an effective amount of another agent that down-regulates GR (such as inhibits GR expression or activity). In some embodiments, at least about 10% (including for example at least about any of 20%, 30%, 40%, 60%, 70%, 80%, 90%, or 100%) metastasis is inhibited. In some embodiments, method of inhibiting metastasis to lymph node is provided. In some embodiments, the taxane composition and the other agent are administered sequentially. In some embodiments, the taxane composition and the other agent are administered simultaneously. In some embodiments, the individual is characterized, by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments of any of the methods, the method comprises a method of inhibiting epithelial-to-mesenchymal transition (EMT) at the in an individual, comprising administering to the individual a) an effective amount of a composition comprising a taxane (such as a composition comprising nanoparticles comprising a taxane (e.g., paclitaxel) and an albumin) and, b) an effective amount of another agent that down-regulates GR (such as inhibits GR expression or activity). In some embodiments, at least about 10% (including for example at least about any of 20%, 30%, 40%, 60%, 70%, 80%, 90%, or 100%) of EMT activity is inhibited. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments of any of the methods, the method comprises a method of reducing tumor size in an individual, comprising administering to the individual a) an effective amount of a composition comprising a taxane (such as a composition comprising nanoparticles comprising a taxane (e.g., paclitaxel) and an albumin) and b) an effective amount of another agent that down-regulates GR (such as inhibits GR expression or activity). In some embodiments, the tumor size is reduced at least about 10% (including for example at least about any of 20%, 30%, 40%, 60%, 70%, 80%, 90%, or 100%). In some embodiments, the taxane composition and the other agent are administered sequentially. In some embodiments, the taxane, composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments of any of the methods, the method comprises a method of prolonging progression-free survival of cancer in an individual, comprising administering to the individual an effective amount of a composition comprising a taxane (such as a composition comprising nanoparticles comprising a taxane (e.g., paclitaxel) and an albumin) and b) an effective amount of another agent that down-regulates GR (such as inhibits GR expression or activity). In some embodiments, the method prolongs the time to disease progression by at least any of 1, 2, 3, 4, 5, 6, 7, 8, 9, 10, 11, or 12 weeks. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments of any of the methods, the method comprises a method of prolonging survival of an individual having cancer, comprising administering to the individual a) an effective amount of a composition comprising a taxane (such as a composition comprising nanoparticles comprising a taxane (e,g., paclitaxel) and an albumin) and b) an effective amount of another agent that down-regulates GR (such as inhibits GR expression or activity). In some embodiments, the method prolongs the survival of the individual by at least any of 1, 2, 3, 4, 5, 6, 7, 8, 9, 10, 11, 12, 18, or 24 months. In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC (such as cortisol) level is used as a basis for selecting the individual for treatment.

In some embodiments of any of the methods, the method comprises a method of reducing AEs and SAEs in an individual having cancer, comprising administering to the individual a) a composition comprising a taxane (such as a composition comprising nanoparticles comprising a taxane (e.g., paclitaxel) and an albumin) and b) an effective amount of another agent that down-regulates GR (such as inhibits GR expression or activity). In some embodiments, the taxane composition and the other agent are administered sequentially. In some embodiments, the taxane composition and the other agent are administered simultaneously. In some embodiments, the individual is characterized by a high GR level (such as expression or activity level) and/or a high GC (such as cortisol) level (such as secretion or activity level). In some embodiments, a high GR and/or GC such as cortisol) level is used as a basis for selecting the individual for treatment.

In some embodiments of any of the methods described herein, the method is predictive of and/or results in an objective response (such as a partial response or complete response).

In some embodiments of any of the methods described herein, the method is predictive of and/or results in improved quality of life.

In some embodiments, a lower amount of each pharmaceutically active compound is used as part of a combination therapy compared to the amount generally used for individual therapy. In some embodiments, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. For example, the use of a small amount of pharmaceutically active compound may result in a reduction in the number, severity, frequency, or duration of one or more side-effects associated with the compound.

The methods described herein can be used for and/or predictive of any one or more of the following purposes: alleviating one or more symptoms of cancer, delaying progressing of cancer, shrinking tumor size, inhibiting tumor growth, prolonging overall survival, prolonging progression free survival, preventing or delaying tumor metastasis, reducing (such as eradiating) preexisting tumor metastasis, reducing incidence or burden of preexisting tumor metastasis, or preventing recurrence.

### GR and GR Responsive Molecules

Glucocorticoid Receptor (GR) belongs to the nuclear receptor superfamily of transcription factors that can sense steroid, hormones and other molecules. In humans, GR is encoded by a single gene named NR3C1. The RNA transcription product of human NR3C1 can undergo alternative splicing generate GR isoforms, including five characterized isoforms: GRα, GRβ, GRγ, GR-P and GR-A. The mRNA of GRα isoform further undergoes alternative translation initiation in exon 2, generating eight additional isoforms of GR with truncated N-termini (GRα-A, GRα-B, GRα-C1, GRα-C2, GRα-C3, GRα-D1, GRα-D2, GRα-D3). As used herein, “glucocorticoid receptor (GR)” refers to the predominant GR isoform, or the GR isoform(s) that binds to and its function regulated by glucocorticoids, such as the GRα isoform in humans. In some embodiments, GR expression is the RNA transcript of a GR-encoding gene (such as the GRα isoform mRNA of human NR3C1), or the protein translated based on the GR RNA transcript (such as the human GRα protein). In some embodiments, GR expression is the total GR protein level, GR protein level in the nucleus, or level of active form(s) of phosphorylated GR protein (such as phosphorylation at serine 211 and serine 226 of human GR).

GR is a modular protein containing an N-terminal transactivation domain (NTD), a central DNA-binding domain (DBD), a C-terminal ligand-binding domain (LBD), and a flexible “hinge region” separating the DBD and the LBD. The NTD has strong transcriptional activation function (AF1), which allows for the recruitment of co-regulators (such as other transcription factors) and transcription machinery. The DBD has two zinc finger motifs that bind specific DNA sequences, called glucocorticoid response elements (GREs), in the promoter or intragenic regions of target genes. The DBD domain is also involved in dimerization of GR, an important event in transactivation or transrepression of target genes. Two nuclear localization signal sequences are found in the DBD and LBD respectively, which can be exposed in GC-bound GR state to trigger nuclear translocation of the GR-GC complex by importin.

GR in its inactive state remains in the cytoplasm, and is usually associated with cytoplasmic chaperones. Upon GC binding to the LBD of a cytoplasmic GR, a GR-GC complex is formed, and the GR is activated by undergoing a conformational change, which promotes dissociation of the cytoplasmic chaperones from the GR, and triggers translocation of the GR-GC complex to the nucleus. Activated GR exerts its pro-survival (e.g., anti-apoptotic), and anti-inflammatory signaling activities through two main mechanisms: non-genomic mechanism and the genomic mechanism. The non-genomic mechanism is mediated by membrane-bound or cytoplasmic GR-GC complex, which is phosphorylated upon activation and elicits rapid (within minutes) downstream actions by activating signal transduction pathways. Although the genomic mechanism involving transcriptional and/or translational regulation usually takes a few hours, GR signaling activities mediated by the genomic mechanism via the nuclear GR-GC complex is much more profound.

In the nucleus, GR enhances or represses transcription of target genes by direct binding to GREs, by tethering itself to other transcription factors, or in a composite manner by direct binding to GRE and interaction with other transcription factors, such as c-Jun of the AP-1 family of transcription factors. Whether GR binding to the GRE upstream of a target gene results in transactivation or transrepression of the target gene depends on multiple factors, such as nature of the GRE (i.e. whether the GRE allows dimerization of GR), recruitment or interaction with other transcription factors, or cell type (such as immune cells or non-immune cells). The direct transcriptional targets of GR include transcription factors that can further transcriptionally enhance or repress indirect GR-responsive genes, along with signaling factors that can activate or inactivate downstream indirect GR-responsive genes.

As used herein, GR-responsive genes include both direct transcriptional targets of GR and indirect GR-responsive genes of the genomic GR-signaling mechanism, as well as direct or indirect targets of GR of the non-genomic GR-signaling mechanism, according to the descriptions above. As used herein, a “GR-responsive molecule” refers to a GR-responsive gene, the product of a GR-responsive gene, or the derivative of a GR-responsive gene or gene product thereof, such as a nucleic acid (DNA or RNA), a protein, or a naturally modified nucleic acid or protein thereof corresponding to the GR-responsive gene. Activation (or inhibition) of GR leads to, or is correlated with a unidirectional change in the level or activity of a GR-responsive molecule, including, but not limited to, binding of GR to the GRE of the GR-responsive gene, methylation/demethylation or chromatin remodeling of the promoter of the GR-responsive gene, other epigenetic modification to the GR-responsive gene, increase/decrease in RNA transcript(s) of the GR-responsive gene (such as mRNA), increase/decrease in the protein product(s) of the GR-responsive gene, phosphorylation/dephosphorylation of the GR-responsive gene product, ubiquitination/de-ubiquitination of the GR-responsive gene product, other post-translational modification of the GR-responsive gene product, or any combination thereof.

GR-responsive molecules can be classified into two broad categories, GR-activated molecules and GR-repressed molecules. In some embodiments, the level (such as expression or activity) of GR-activated molecules positively correlates with the level (such as expression or activity) of GR. In some embodiments, the level (such as expression or activity) of GR-repressed molecules negatively correlates with the level (such as expression or activity) of GR.

GR-responsive molecules are known in the art (see for example, U.S. Pat. No. 8,710,035 B2; Wu et al. (2004) “Microarray analysis reveals glucocorticoid-regulated survival genes that are associated with inhibition of apoptosis in breast epithelial cells.” Cancer Res 64:1757-1764; and Wang J C et al. (2004) “Chromatin immunoprecipitation (ChIP) Scanning identifies primary glucocorticoid receptor target genes” PNAS 101(44): 15603-15608, incorporated herein by reference in their entireties). For example, in breast cancer cells, GR-responsive molecules include, but not limited to, GR-activated molecules corresponding to DUSP1, SGK1, SMARCA2, PTGDS, MCL1, DPYSL2, STOM, LAPTM5, NNMT, SERPINF1, NRIP1, WIPF1, BIN1, IL1R1, ST3GAL5, SEMA4D, MAP3K5, SMARCA2, DPT, BIRC3, PTGDS, PHF15, MAOA, TFPI, SLC46A3, PIAS1, ACSL5, SESN1, C14orf139, and LBH; and GR-repressed molecules corresponding to SFN, SPP1, and ERBB2. For example, in human lung adenocarcinoma cells, GR-responsive molecules include, but not limited to, GR-activated molecules corresponding to GCL20, GILZ, FLAP, and THBD in inflammation pathways; GADD45B, HIAP1, Kip2/p57, MFGE8, S100P, SLUG, hSPRY1, and TNFAIP3 in cell growth and apoptosis pathways; AKAP13, ANKRD1, CDC42EP3, CDC42EP3, CPEB4, DNER, EHM2, ET-2, FKBP5, FGD4, IHPK3, IRS2, POU5F1, PP1R14C, RGS2, SEC14L1, and TGFBR3 in signal transduction pathways; ANGPTL4, B3GNT5, EKI2, and MGAM in metabolic pathways; ENaCa, MT-1I, SLC19A2, SLC26A2, and Stomatin in transport processes; and ABHD2, CTEN, FLJ11127, FLJ20371, GPR115, GPR153, LOC144100, LRRC8, MCJ, PPG/Serglycin, SDPR, SPINK5L3, and TMG4 in other or unknown cellular pathways or functions. GR-responsive molecules in human lung adenocarcinoma cells also include, but not limited to, GR-repressed molecules corresponding to COX-2, PDE4B/2, GCL2, and IL-11 in inflammation pathways; Cullin 1, CAP3/IP9, FGFBP1, and TRIP-Br2 in cell growth and apoptosis pathways; ARL8, BHLHB2, ENC1, GEM, RDC1, SNK and ZIC2 in signal transduction pathways; and AMIGO2, CG1/XP28, KIAA1376, FLJ22761, NAV3 and PMP2 in other or unknown cellular pathways or functions. The inventors further found that GR-responsive molecules include GR-activated molecules (such as protein or phosphorylated protein) corresponding to SGK1, MKP1, MCL-1, Bcl2, BCLxL, AKT, MAP Tau, FN1, SERPINE1, SLUG, RGS2, KRT7, MME, IL1R1, JAK2, and CEBPD; and GR-repressed molecules (such as protein or phosphorylated protein) corresponding to JNK1 and c-Jun (including AP-1), IL6, LIF, and TNFRSF11B. Other exemplary GR-responsive molecules include, but are not limited to, GR-activated molecules corresponding to anti-apoptotic/anti-inflammatory genes IKK, and IκB (including active forms of phosphorylated IκB); and GR-repressed molecules corresponding to pro-inflammatory gene NF-κB, pro-apoptotic gene p53, JNKK/Sek1, and JNK/SAPK. In some embodiments of any of the methods described herein, the GR responsive molecule is selected from the group consisting of SGK1, MKP1, MCL1, SAP30, DUSP1, SMARCA2, PTGDS, TNFRSF9, SFN, LAPTM5, GPSM2, SGRT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG, SERPINE1, RGS2, KRT7, MME, JAK2, CEBPD, IL6, LIF, and TNFRSF11B. In some embodiments according to any of the methods described herein, the GR activity level is determined by measuring the level (such as expression or activity) of any one or any combination of the exemplary GR-responsive molecules described herein.

As used herein, “GR activity” comprises enhancement or repression of any GR responsive molecule (including epigenetic, transcriptional, translational or post-translational regulations or modulations), or any combination thereof. Additionally, GR activity comprises the downstream cellular and physiological effects in response to the modifications of the GR-responsive genes, including, but not limited to, apoptosis, cell growth, proliferation, inflammation (such as release of cytokines), signal transduction, metabolism, stress response (such as cell cycle arrest, microtubule polymerization/depolymerization, or transport/clearance of small molecules).

In general, GR activity leads to enhanced cell survival in epithelial cells; and reduced apoptosis, cell toxicity, and immune response in non-immune cells; GR activity also leads to reduced cell growth, proliferation, and survival; and enhanced apoptosis, and cell toxicity in immune cells. The inventors further found that GR activity also induces regulatory genes in epithelial-mesenchymal transition (EMT). In some embodiments according to any of the methods described herein, the GR activity is enhancement (such as up-regulation) of a GR-activated molecule, wherein the GR-activated molecule directly or indirectly inhibits apoptosis, stress response, or inflammatory response (such as secretion of cytokines), and/or promotes cell proliferation, protection, survival, or EMT. In some embodiments, the GR activity is suppression (such as down-regulation) of a GR-repressed molecule, wherein the GR-repressed molecule directly or indirectly promotes apoptosis, stress response, or inflammatory response (such as secretion of cytokines), and/or inhibits cell proliferation, protection, survival, or EMT.

### Methods of Determining Levels of GR

The methods described herein in some embodiments comprise determining the level of one or more GRs in an individual. In some embodiments, the level is the activity level of a GR in a sample, and the activity level can encompass, for example, a measure of the level (such as expression or activity) of a GR-responsive molecule. In some embodiments, the level is an expression level that correlates to the activity level. In some embodiments, the level is a measure of a protein present in a cell (for example inside the cell (including membrane-associated, cytoplasmic and nuclear portions) or in the nucleus), a sample, or a tumor. In some embodiments, the level is a measure of a phosphorylated GR in an active form (such as phosphorylated at serine 211 and/or serine 226 in human GR protein). In some embodiments, a level is a measure of a nucleic acid present in a cell, a sample, or a tumor. In some embodiments, the level is based on a mutation or polymorphism in the GR gene that correlates with the protein or mRNA level of a GR. In some embodiments, the level is based on epigenetic modification (such as chromatin markers or methylation) of the GR gene that correlates with the protein or mRNA level of a GR. In some embodiments, the level is the protein expression level. In some embodiments, the level is the mRNA level.

The levels of GRs can be determined by methods known in the art. See, for example, Spratlin et al., Cancers 2010, 2, 2044-2054; Santini et al., Current Cancer Drug Targets, 2011, 11, 123-129; Kawada et al. J. Hepatobiliary Pancreat. Sci., 2012, 19:17-722; Morinaga et al., Ann. Surg. Oncol., 2012, 19, S558-S564. See also US Pat. Pub. No. 2013/0005678, and U.S. Pat. No. 8,710,035.

Levels of GR in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. Bodily fluids include, but are not limited to, blood, lymph, saliva, semen, peritoneal fluid, cerebrospinal fluid, breast milk, and pleural effusion. In some embodiments, the sample is a blood sample which includes, for example, platelets, lymphocytes, polymorphonuclear cells, macrophages, and erythrocytes.

In some embodiments, the sample is a tumor tissue, normal tissue adjacent to said tumor, normal tissue distal to said tumor, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing cancer cells. In a further embodiment, the biopsy is a fine needle aspiration of pancreatic cancer cells. In a further embodiment, the biopsy is laparoscopy obtained pancreatic cancer cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes the GR. In some embodiments, a biopsy is taken to determine whether an individual has cancer and is then used as a sample. In some embodiments, the sample comprises surgically obtained tumor cells. In some embodiments, samples may be obtained at different times than when the determining of GR levels occurs.

In some embodiments, the sample comprises a circulating metastatic pancreatic cancer cell. In some embodiments, the sample is obtained by sorting pancreatic circulating tumor cells (CTCs) from blood. In a further embodiment, the CTCs have detached from a primary tumor and circulate in a bodily fluid. In yet a further embodiment, the CTCs have detached from a primary tumor and circulate in the bloodstream. In a further embodiment, the CTCs are an indication of metastasis.

In some embodiments, the protein expression level of the GR or one or more GR-responsive molecules (as a measure of GR activity) is determined. In some embodiments, a Western blot assay is used to determine the protein expression level of the GR or the one or more GR-responsive molecules. In some embodiments, an enzyme-linked immunosorbent assay (ELISA) is used to determine the protein expression level of the GR or the one or more GR-responsive molecules. In some embodiments, a protein level of the GR or the one or more GR-responsive molecules in a sample are normalized (such as divided) by the protein level of a housekeeping protein (such as glyceraldehyde 3-phosphate dehydrogenase, or GAPDH) in the same sample to determine the protein level of the GR or the one or more GR-responsive molecules. In some embodiments, the mRNA level of the GR or one or more GR responsive molecule (as a measure of GR activity) is determined. In some embodiments, a reverse-transcription (RT) polymerase chain reaction (PGR) assay (including a quantitative RT-PGR assay) is used to determine the mRNA level of the GR or the one or more GR-responsive molecules. In some embodiments, a gene chip or next-generation sequencing methods are used to determine the expression level of the GR and/or the one or more GR responsive molecules (such as mRNA level). In some embodiments, an mRNA level of the GR or the one or more GR-responsive molecules in a sample are normalized (such as divided) by the mRNA level of a housekeeping gene (such as GAPDH) in the same sample to determine the mRNA level of the GR or the one or more GR-responsive molecules. In some embodiments, the level of the GR or one or more GR-responsive molecules (as a measure of GR activity) is determined by an immunohistochemistry method.

The levels of a GR may be a high level or a low level as compared to a control sample. In some embodiments, the level of the GR in an individual is compared to the level of the GR in a control sample. In some embodiments the level of the GR in an individual is compared to the level of the GR in multiple control samples. In some embodiments, multiple control samples are used to generate a statistic that is used to classify the level of the GR in an individual with cancer.

In some embodiments, the DNA copy number is determined, and a high DNA copy number for the gene encoding the GR (for example a high DNA copy number as compared to a control sample) is indicative of a high level of the GR.

The classification or ranking of the GR level (i.e., high or low) may be determined relative to a statistical distribution of control levels. In some embodiments, the classification or ranking is relative to a control sample, such as a normal tissue (e.g. peripheral blood mononuclear cells), or a normal epithelial cell sample (e.g. a buccal swap or a skin punch) obtained from the individual. In some embodiment, the level of the GR is classified or ranked relative to a statistical distribution of control levels. In some embodiments, the level of the GR is classified or ranked relative to the level from a control sample obtained from the individual.

Control samples can be obtained using the same sources and methods as non-control samples. In some embodiments, the control sample is obtained from a different individual (for example an individual not having cancer, an individual having a benign or less advanced form of a disease corresponding to the cancer, and/or an individual sharing similar ethnic, age, and gender identity). In some embodiments when the sample is a tumor tissue sample, the control sample may be a non-cancerous sample from the same individual. In some embodiments, multiple control samples (for example from different individuals) are used to determine a range of levels of GRs in a particular tissue, organ, or cell population.

In some embodiments, the control sample is a cultured tissue or cell that has been determined to be a proper control. In some embodiments, the control is a cell that does not express the GR. In some embodiments, a clinically accepted normal level in a standardized test is used, as a control level for determining the GR level. In some embodiments, the reference level of GR or GR responsive molecule in the individual is classified as high, medium or low according to a scoring system, such as an immunohistochemistry-based scoring system.

In some embodiments, the GR level is determined by measuring the level of a GR in an individual and comparing to a control or reference (e.g., the median level for the given patient population or level of a second individual). For example, if the level of a GR for the single individual is determined to be above the median level of the patient population, that individual is determined to have high expression of the GR. Alternatively, if the level of a GR for the single individual is determined to be below the median level of the patient population, that individual is determined to have low expression of the GR. In some embodiments, the individual is compared to a second individual and/or a patient population which is responsive to treatment. In some embodiments, the individual is compared to a second individual and/or a patient population which is not responsive to treatment. In some embodiments, the levels are determined by measuring the level of a nucleic acid encoding a GR or a GR responsive molecule. For example, if the level of an mRNA encoding a GR for the single individual is determined to be above the median level of the patient population, that individual is determined to have a high level of an mRNA encoding the GR. Alternatively, if the level of mRNA encoding the GR for the single individual is determined to be below the median level of the patient population, that individual is determined to have a low level of an mRNA encoding the GR.

In some embodiments, the control level of a GR is determined by obtaining a statistical distribution of GR levels. In some embodiments, the level of the GR is classified or ranked relative to control levels or a statistical distribution of control levels.

In some embodiments, bioinformatics methods are used for the determination and classification of the levels of the GR (including the levels of GR-responsive molecules as a measure of the GR level). Numerous bioinformatics approaches have been developed to assess gene set expression profiles using gene expression profiling data. Methods include but are not limited to those described in Segal, E. et al, Nat. Genet. 34:66-176 (2003); Segal, E. et al. Nat. Genet. 36:1090-1098 (2004); Barry, W. T. et al. Bioinformatics 21:1943-1949 (2005); Tian, L. et al. Proc Nat'l Acad Sci USA 102:13544-13549 (2005); Novak B A and Jain A N. Bioinformatics 22:233-41 (2006); Maglietta R et al. Bioinformatics 23:2063-72 (2007); Bussemaker H J, BMC Bioinformatics 8 Suppl 6:S6 (2007).

In some embodiments, the control level is a pre-determined threshold level. In some embodiments, the pre-determined threshold level is based on cross-referencing GR levels of a plurality of control samples determined by an assay (such as RT-PCR, qRT-PGR, Western blot, ELISA, gene chip, next-generation sequencing, or immunohistochemistry) to the GR levels in the Cancer Genome Atlas (TGCA) cohort according to the Pan-Cancer analysis (The Cancer Genome Atlas Research Network et al., 2013). In some embodiments, the GR levels of the plurality of control samples are correlated to GR levels of corresponding samples in the TCGA cohort (such as corresponding samples having the same cancer type, stage, cell origin, and/or patient demographics). In some embodiments, the pre-determined threshold level corresponds to the median GR level of the corresponding samples in the TGCA cohort. In some embodiments, the pre-determined threshold level corresponds to more than about any of 6, 7, 8, 9, 10, 11, 12, 13, or 14 relative expression units according to the Pan Cancer analysis of the TCGA cohort.

In some embodiments, mRNA level is determined, and a low level is an mRNA level less than about any of 1.1, 1.2, 1.3, 1.5, 1.7, 2, 2.2, 2.5, 2.7, 3, 5, 7, 10, 20, 50, 70, 100, 200, 500, 1000 times or more than 1000 times to that of what is considered as clinically normal or to the level obtained from a control. In some embodiments, a high level is an mRNA level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times to that of what is considered as clinically normal or to the level obtained from a control.

In some embodiments, protein expression level is determined, for example by Western blot or an enzyme-linked immunosorbent assay (ELISA). For example, the criteria for low or high levels can be made based on the total intensity of a band on a protein gel corresponding to the GR (or a GR-responsive molecule) that is blotted by an antibody that specifically recognizes the GR protein (or the GR-responsive molecule), and normalized (such as divided) by a band on the same protein gel of the same sample corresponding to a housekeeping protein (such as GAPDH) that is blotted by an antibody that specifically recognizes the housekeeping protein (such as GAPDH). In some embodiments, the protein level is low if the protein level is less than about any of 1.1, 1.2, 1.3, 1.5, 1.7, 2, 2,2, 2.5, 2.7, 3, 5, 7, 10, 20, 50, 70, 100 times or more than 100 times to that of what is considered as clinically normal or to the level obtained from a control. In some embodiments, the protein level is high if the protein level is more than about any of 1.1, 1.2, 1.3, 1.5, 1.7, 2, 2.2, 2.5, 2.7, 3, 5, 7, 10, 20, 50, 70, 100 times or more than 100 times to that of what is considered as clinically normal or to the level obtained from a control. In some embodiments, the GR protein level is high if the GAPDH-normalized GR protein level is more than about any of 2, 3, 4, 4.5, 5, 5.5, 6, 7, 8, 9, 10, 11, 12, 13, 14, or more than 14. In some embodiments, the GR protein level is low if the GAPDH-normalized GR protein level is less than about any of 2, 3, 4, 4.5, 5, 5.5, 6, 7, 8, 9, 10, 11, 12, 13, 14, or more than 14.

In some embodiments, protein expression level is determined, for example by immunohistochemistry. For example, the criteria for low or high levels can be made based on the number of positive staining cells and/or the intensity of the staining, for example by using an antibody that specifically recognizes the GR protein. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than a positive control staining. In some embodiments, the level is high if more than about 40%, 45%, 50%, 55%, 60%, 65%, 70%, 75%, 80%, 85%, or 90%, cells have positive staining.

In some embodiments, the level is high if the staining is as intense as positive control staining In some embodiments, the level is high if the staining is 80%, 85%, or 90% as intense as positive control staining.

In some embodiments, the scoring is based on an “H-score” as described in US Pat. Pub. No. 2013/0005678. An H-score is obtained by the formula: 3×percentage of strongly staining cells+2×percentage of moderately staining cells+percentage of weakly staining cells, giving a range of 0 to 300.

In some embodiments, strong staining, moderate staining, and weak staining are calibrated levels of staining, wherein a range is established and the intensity of staining is binned within the range. In some embodiments, strong staining is staining above the 75th percentile of the intensity range, moderate staining is staining from the 25th to the 75th percentile of the intensity range, and low staining is staining is staining below the 25th percentile of the intensity range. In some aspects one skilled in the art, and familiar with a particular staining technique, adjusts the bin size and defines the staining categories.

In some embodiments, the label high GR staining is assigned where greater than 50% of the cells stained exhibited strong reactivity, the label no GR staining is assigned where no staining was observed in less than 50% of the cells stained, and the label low GR staining is assigned for all of other cases.

In some embodiments, the assessment and scoring of the GR level in a sample, patient, etc., is performed by one or more experienced clinicians, i.e., those who are experienced with GR expression and GR staining patterns. For example, in some embodiments, the clinician(s) is blinded to clinical characteristics and outcome for the samples, patients, etc, being assessed and scored.

Further provided herein are methods of directing treatment of a cancer by delivering a sample to a diagnostic tab for determination of GR levels; providing a control sample with a known level of a GR; providing an antibody to a GR (e.g., GR antibody) or an antibody to a GR-responsive molecule; individually contacting the sample and control sample with the antibody, and/or detecting a relative amount of antibody binding, wherein the level of the sample is used to provide a conclusion that a patient should receive a treatment with any one of the methods described herein. Also provided herein are methods of directing treatment of a disease, further comprising reviewing or analyzing data relating to the presence (or level) of a GR (or GR-responsive molecule) in a sample; and providing a conclusion to an individual about the likelihood or suitability of the individual to respond to a treatment, a health care provider or a health care manager, the conclusion being based on the review or analysis of data. In one aspect of the invention a conclusion is the transmission of the data over a network.

### Methods of Determining Levels of GC

The methods described herein in some embodiments comprise determining the level of one or more GCs (such as cortisol) in an individual.

Generally, the predominant GC that binds and activated GR in humans is cortisol. Cortisol is secreted by the adrenal cortex in response to conditions, such as stress, in humans. Cortisol secretion in human is stimulated by another hormone, named adrenocorticotropic hormone (ACTH). Cortisol is metabolized in peripheral tissues in human to a different GC named cortisone by the enzyme 11-beta-steroid dehydrogenase. Cortisone is essentially an inactive metabolite of cortisol, as cortisone cannot activate GR activity with high efficacy.

In some embodiments, the method comprises determining the level of one or more endogenous GCs in the individual. In some embodiments, the one or more GCs comprise cortisol, its metabolite, its precursor, or its stimulator (such as ACTH). In some embodiments, the method comprises determining the level of one or more GCs of exogenous sources, such as from medications (including those related to cancer therapy and those not related to cancer therapy) or dietary supplements, in the individual. In some embodiments, the method comprises determining the level of one or more GCs from both endogenous sources and exogenous sources from an individual.

In some embodiments, the GC level (including the level of one or more GCs) is GC secretion, for example, the amount of the GC in a sample, tissue or cell in the individual. In some embodiments, the level of GC is GC activity, such as binding to GR or activation of GR activity.

Cortisol and other endogenous GCs are widely distributed among human tissues, and their levels can be measured in many samples, such as blood (including plasma and serum), urine, and saliva. In serum, about 90-95% of cortisol is bound to proteins, such as the corticosteroid binding globulin (CBG). “Free GC” (such as free cortisol) refers to GC (such as cortisol) not bound to proteins. Cortisol in the urine is essentially free cortisol. In saliva, about 67% of cortisol is free. There is generally good correlation between cortisol measurements in saliva and serum.

In some embodiments, the GC level is the total GC level, including protein-bound GC and free GC. In some embodiments, the GC level is the free GC level. In some embodiments, the GC level in a blood sample (such as serum or plasma) is determined. In some embodiments, the GC level in a urine sample is determined. In some embodiments, the GC level in a saliva sample is determined.

The levels of GCs (such as cortisol) can be determined by methods known in the art. See, for example, Lundstrom S. et al. (2003) “Symptoms in advanced cancer: relationship to endogenous cortisol levels.” Palliative Medicine 17:503-508; Kirschbaum C. and Hellhammer D H. (1989) “Salivary cortisol in psychobiological research: an overview.” Neuropsychobiology 22: 150-169; Guber H A and Farag A F. (2011) “Evaluation of endocrine function.” In: McPherson R A, Pincus M R, eds. “Henry's clinical diagnosis and management by laboratory methods.” 22^{nd} ed. Philadelphia, Pa.: Elsevier Saunders: chap 24; Stewart P M, Krone N P. (2011) “The adrenal cortex.” In: Melmed S, et al. eds. “Williams Textbook of Endocrinology.” 12^{th} ed. Philadelphia: chap 15.

Exemplary methods for quantifying GC secretion, such as cortisol secretion levels, include, but are not limited to, immunoassays (such as radioimmunoassay and enzyme immunoassay), and gas or liquid chromatography (such as those coupled with mass spectrometry). In some embodiments, the GC secretion is determined using an immunoassay that uses an antibody directed against certain parts or the entirety of the GC (such as cortisol). Commercial assay kits, such as the CAYMAN™ Cortisol EIA Kit, can be used to determine the GC secretion level. In some embodiments, equilibrium dialysis can be used to obtain the protein-free fraction of GC from a sample, such as a serum sample.

The level of GCs (such as cortisol) in an individual naturally varies according to a circadian rhythm, with samples taken from morning having the highest GC level and samples taken from evenings having the lowest GC level. In some embodiments, the GC level is determined based on a sample taken in the morning (such as 9 am or shortly after the individual wakes up). In some embodiments, the GC level is based on a sample taken in the evening (such as 9 pm, or shortly before the individual goes to sleep). In some embodiment, the GC level is determined based on a cumulative sample over a 24 hour period, such as a 24 hour urine sample. The level of GCs also varies according to activity levels of an individual, and/or stress level of an individual. In some embodiments, the level of GCs is based on a sample for the individual, wherein the individual is advised not to engage in any physical activity for any of 30 minutes, 1 hour, 2 hours, 4 hours, or more prior to taking the sample.

Levels of GC (such as cortisol) in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. Bodily fluids include, but are not limited to, blood, lymph, saliva, semen, peritoneal fluid, cerebrospinal fluid, breast milk, and pleural effusion. In some embodiments, the sample is a blood sample which includes, for example, platelets, lymphocytes, polymorphonuclear cells, macrophages, and erythrocytes.

In some embodiments, the sample is a tumor tissue, normal tissue adjacent to said tumor, normal tissue distal to said tumor, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing cancer cells. In a further embodiment, the biopsy is a fine needle aspiration of pancreatic cancer cells. In a further embodiment, the biopsy is laparoscopy obtained pancreatic cancer cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes the GC. In some embodiments, a biopsy is taken to determine whether an individual has cancer and is then used as a sample. In some embodiments, the sample comprises surgically obtained tumor cells. In some embodiments, samples may be obtained at different times than when the determining of GC levels occurs.

In some embodiments, the sample comprises a circulating metastatic pancreatic cancer cell. In some embodiments, the sample is obtained by sorting pancreatic circulating tumor cells (CTCs) from blood. In a further embodiment, the CTCs have detached from a primary tumor and circulate in a bodily fluid. In yet a further embodiment, the CTCs have detached from a primary tumor and circulate in the bloodstream. In a further embodiment, the CTCs are an indication of metastasis.

The level of a GC (such as cortisol) may be a high level or a low level as compared to a control sample. In some embodiments, the level of the GC in an individual is compared to the level of the GC in a control sample. In some embodiments the level of the GC in an individual is compared to the level of the GC in multiple control samples. In some embodiments, multiple control samples are used to generate a statistic that is used to classify the level of the GC in an individual with cancer.

The classification or ranking of the GC (such as cortisol) level (i.e., high or low) may be determined relative to a statistical distribution of control levels. In some embodiment, the level of the GC is classified or ranked relative to a statistical distribution of control levels. In some embodiments, the level of the GC is classified or ranked relative to the level from a control sample obtained from the individual.

Control samples can be obtained using the same sources and methods as non-control samples. In some embodiments, the control sample is obtained from a different individual (for example an individual not having cancer, an individual having a benign or less advanced form of a disease corresponding to the cancer, and/or an individual sharing similar ethnic, age, and gender identity). In some embodiments when the sample is a tumor tissue sample, the control sample may be a non-cancerous sample from the same individual. In some embodiments, multiple control samples (for example from different individuals) are used to determine a range of levels of GCs in a particular tissue, organ, or cell population.

In some embodiments, the control sample is a cultured tissue or cell that has been determined to be a proper control. In some embodiments, a clinically accepted normal level in a standardized test is used as a control level for determining the GC (such as cortisol) level. For example, the clinically accepted normal level of free cortisol in a human blood plasma sample taken at about 9 am in the morning is about 5-25 μg/dL. For example, the clinically accepted normal level of free cortisol in a human blood plasma sample taken at midnight is about 2.9-13 μg/dL. For example, the clinically accepted normal level of free cortisol in a human 24 hour urine sample is about 100 μg/dL in adults, about 5-55 μg/dL in teens, or 2-27 μg/dL in children. In some embodiments, a GC level (such as cortisol level) is a high level if the GC level determined in a GC blood test is at least about any of 1.3, 1.5, 1.7, 2, 3, 4, 5, or more times that of a clinically accepted normal level for the GC blood test. In some embodiments, a GC level (such as cortisol level) is a low level if the GC level determined in a GC test (such as a blood test or a urine test) is less than about any of 1.3, 1.5, 1.7, 2, 3, 4, 5, or more times that of a clinically accepted normal level for the GC test. In some embodiments, the GC level determined in a GC blood test is a high level if the GC level determined in a GC blood test based on a sample taken in the morning (such as 9 am) is at least about any of 1.3, 1.5, 1.7, 2, 3, 4, 5, or more times the clinically accepted normal level (for example, about 5-23 of free cortisol in adults and children). In some embodiments, the GC level (such as cortisol level) in the individual is a high level if the GC level is determined in a 24-hour GC urine test of at least about any of 1.3, 1.5, 1.7, 2, 3, 4, 5, or more times the clinically accepted normal level (for example, less than about 100 μg/dL or cortisol in adults, about 5-55 μg/dL in teens, or 2-27 μg/dL in children).

In some embodiments, the GC (such as cortisol) level is determined by measuring the level of a GC in an individual and comparing to a control or reference (e.g., the median level for the given patient population or level of a second individual). For example, if the level of a GC for the single individual is determined to be above the median level of the patient population, that individual is determined to have high expression of the GC. Alternatively, if the level of a GC for the single individual is determined to be below the median level of the patient population, that individual is determined to have low expression of the GC. In some embodiments, the individual is compared to a second individual and/or a patient population which is responsive to treatment. In some embodiments, the individual is compared to a second individual and/or a patient population which is not responsive to treatment. In some embodiments, the reference level of a GC is determined by obtaining a statistical distribution of GC levels.

In some embodiments, the assessment and scoring of the GC (such as cortisol) level in a sample, patient, etc., is performed by one or more experienced clinicians, i.e., those who are experienced with GC secretion or GC activity patterns. For example, in some embodiments, the clinician(s) is blinded to clinical characteristics and outcome for the samples, patients, etc. being assessed and scored.

Further provided herein are methods of directing treatment of a cancer by delivering a sample to a diagnostic tab for determination of GC (such as cortisol) levels; providing a control sample with a known level of a GC (such as cortisol); providing an antibody to a GC (e.g., cortisol antibody); individually contacting the sample and control sample with the antibody, and/or detecting a relative amount of antibody binding, wherein the level of the sample is used to provide a conclusion that a patient should receive a treatment with any one of the methods described herein. Also provided herein are methods of directing treatment of a disease, further comprising reviewing or analyzing data relating to the presence (or level) of a GC (such as cortisol) in a sample; and providing a conclusion to an individual about the likelihood or suitability of the individual to respond to a treatment, a health care provider or a health care manager, the conclusion being based on the review or analysis of data. In one aspect of the invention a conclusion is the transmission of the data over a network.

### GR Down-Regulators

The methods described herein in some embodiments comprise administration of another agent that down-regulates GR (also referred to as “GR down-regulators”). “A down-regulator” is a molecule that reduces (including inhibits) the level of a gene (such as expression or activity) when administered to an individual. In some embodiments, the GR down-regulator reduces the GR level (such as GR expression or activity) to less than about any of 10%, 20%, 30%, 40%, 50%, 60%, 70%, 80%, or 90% of the GR level prior to administration of the GR down-regulator. In some embodiments, a GR down-regulator is a molecule that reduces the cellular response to an elevated GR level (such as GR expression or activity) by about any of 10%, 20%, 30%, 40%, 50%, 60%, 70%, 80%, 90%, or more. In some embodiments, the GR down-regulator reduces the GR level in all tissues and cells in the individual. In some embodiments, the GR down-regulator preferentially reduces the GR level in tumor cells without significantly altering the GR level in normal cells in the individual.

The GR down-regulator may be of any suitable molecular modality, including, but not limited to, nucleic acids (such as aptamer, mRNA, RNAi agents, etc.), peptides, proteins, antibodies, small molecules, and compositions for gene knock-down (such as TALEN, Zinc Finger Nuclease, CRISPR/Cas9, etc.).

In some embodiments, the GR down-regulator is an inhibitor of GR expression. For example, the inhibitor of GR expression reduces the amount of GR, such as RNA transcript (for example, mRNA) or protein. In some embodiments, the inhibitor of GR expression reduces the amount of GR by about any of 10%, 20%, 30%, 40%, 50%, 60%, 70%, 80%, 90%, or more. In some embodiments, the inhibitor of GR expression is a therapeutic oligonucleotide, such as DNA, RNA, PNA, phosphorodiamidate morpholino oligomers, other chemically modified oligonucleotide, or hybrids of any types of oligonucleotides thereof, such as those described in Dias N. and Stein C A. (2002) “Antisense Oligonucleotides: Basic Concepts and Mechanisms” Molecular Cancer Therapeutics 1:347. In some embodiments, the therapeutic oligonucleotide is an antisense oligonucleotide or siRNA that bind to a GR RNA (such as GR mRNA), an antigene oligonucleotide that binds to GR gene, or an oligonucleotide aptamer or decoy that reduces the amount of GR expression, such as those described in Goodchild J. (2011) “Therapeutic oligonucleotides,” Methods Mol Biol. 764: 1-15. In some embodiments, the inhibitor of GR expression is a molecule that inhibits phosphorylation of GR (such as at residue 211 or residue 226 of human GR), promotes degradation of GR protein, or reduces GR protein level in the cell or in the nucleus.

In some embodiments, GR down-regulator is an inhibitor of GR activity. In some embodiments, the inhibitor of GR activity reduces GR activity by about any of 10%, 20%, 30%, 40%, 50%, 60%, 70%, 80%, 90%, or more. In some embodiments, the inhibitor of GR activity reduces GR-induced anti-apoptosis activity by about any of 10%, 20%, 30%, 40%, 50%, 60%, 70%, 80%, 90%, or more. In some embodiments, the inhibitor of GR activity reduces GR-induced EMT activity by about any of 10%, 20%, 30%, 40%, 50%, 60%, 70%, 80%, 90%, or more. In some embodiments, the inhibitor of GR activity is a modulator of one or more than one GR-responsive molecule, such as any of the GR responsive molecule described herein. In some embodiments, the inhibitor of GR activity is a modulator of any one or any combination of GR-responsive molecules selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG,SERPINE1, RGS2, KRT7, MME, JAK2, CEBPD, IL6, LIF, and TNFRSF11B. In some embodiments, the inhibitor of GR activity is a modulator of any one or any combination of GR-responsive molecules selected from the group consisting of GCL20, GILZ, FLAP, THBD, GADD45B, HIAP1, Kip2/p57, MFGE8, S100P, SLUG, hSPRY1, TNFA1P3, AKAP13, ANKRD1, CDC42EP3, CDC42EP3, CPEB4, DNER, EHM2, ET-2, FKBP5, FGD4, HPK3, IRS2, POU5F1, PP1R14C, RGS2, SEC14L1, TGFBR3, ANGPTL4, B3GNT5, EKI2, ENaCa, MT-11, SLC19A2, SLC26A2, ABHD2, CTEN, FLJ11127, FLJ20371, GPR115, GPR153, LOC144100, LRRC8, MCJ, PPG/Serglycin, SDPR, SPINK5L3, TMG4, OX-2, PDE4B/2, GCL2, IL-11, Cullin 1, CAP3/IP9, FGFBP1, TRIP-Br2, ARL8, BHLHB2, ENC1, GEM, RDC1, SNK, ZIC2, AMIGO2, CG1/XP28, KIAA1376, FLJ22761, NAV3 and PMP2. In some embodiments, the inhibitor of GR activity is a modulator of any one or any combination of GR-responsive molecules selected from the group consisting of SGK1, MKP1, MCL-1, Bcl2, BCLxL, AKT, MAP Tau, JNK1, c-Jun, AP-1. In some embodiments, the inhibitor of GR activity is a modulator of any one or any combination of GR-responsive molecules selected from the group consisting of FN1, SLUG,SERPINE1, RGS2, KRT7, MME, IL1R1, JAK2, CEBPD, MCL1, IL6, LIF, and TNFRSF11B. In some embodiments, the inhibitor of GR activity is an inhibitor of SLUG, such as an RNAi agent against SLUG.

In some embodiments, the inhibitor of GR activity is a down-regulator of a GR-activated molecule, such as an inhibitor of a GR-activated molecule expression or an inhibitor of a GR-activated molecule activity. In some embodiments, the inhibitor of GR activity is an up-regulator of a GR-repressed molecule, such as an activator of a GR-repressed molecule expression or an activator of a GR-repressed molecule activity. In some embodiments, the inhibitor comprises a down-regulator of a GR activated molecule, or an activator of a GR-repressed molecule. In some embodiments, the inhibitor of GR activity is a specific kinase inhibitor, such as an inhibitor of SGK-1. In some embodiments, the inhibitor of GR activity is a specific phosphatase inhibitor, such as an inhibitor of MKP-1. In some embodiments, the inhibitor of GR activity is a molecule that inhibits tumor cell proliferation, inhibits EMT, promotes tumor cell apoptosis, or promotes immune response against tumor cells.

In some embodiments, the other agent is a GR antagonist. A GR antagonist is a molecule, which, upon binding to GR, inhibits or dampens GC-mediated GR activity, and does not provoke GR activity by itself. In some embodiments, upon binding to GR, the GR antagonist dampens GC-mediated GR activity by about any of 10%, 20%, 30%, 40%, 50%, 60%, 70%, 80%, 90%, or more. A GR antagonist may bind to the same site on the GR as GC, to an allosteric site on the GR, or to a binding site not normally involved in the biological regulation of the GR's activity. In some embodiments, the GR antagonist is a steroid, such as a steroid that binds to the GC-binding site in the GR. In some embodiments, the GR antagonist is nonsteroidal. In some embodiments, the GR antagonist binds to GR with higher or similar affinity than GC. In some embodiments, the GR antagonist competes with GC for binding to GR.

In some embodiments, the glucocorticoid receptor antagonist is a selective glucocorticoid receptor antagonist. In some embodiments, the GR antagonist does not bind to progesterone receptor (PR) with high affinity (such as lower affinity than about any of 1 μM, 100 μM or 1 mM). In some embodiments, the GR antagonist does not have PR antagonist activity. In some embodiments, the GR antagonist does not bind to androgen receptor (AR) with high affinity (such as lower affinity than about any of 1 μM, 100 μM or 1 mM). In some embodiments, the GR antagonist does not have AR antagonist activity. In some embodiments, the GR antagonist does not bind to PR or AR with high affinity. In some embodiments, the GR antagonist does not have PR or AR antagonist activity. In some embodiments, the GR antagonist is a context-dependent GR antagonist that inhibit GR activity in certain downstream pathways (such as certain target genes), or in certain cell types. Some exemplary selective and context-dependent GR antagonists are known in the art, see for example, arylpyrazole derivatives with substitutions at the C-11 position (such as ligand 15 with a hydroxyphenyl substitution) as described in Wang J C et al. (2006) “Novel arylpyrazole compounds selectively modulate glucocorticoid receptor regulatory activity,” Genes & Development 20:689-699.

In some embodiments, the glucocorticoid receptor antagonist is a non-selective glucocorticoid receptor antagonist, such as mifepristone (RU-486).

Other exemplary GR antagonists are known in the art, see for example, as described by Clark (2008) “Glucocorticoid receptor antagonists” Current Topics in Medicinal Chemistry 8:813-838; Peeters et al. “Differential effects of the new glucocorticoid receptor antagonist ORG34517 and RU486 (mifepristone) on glucocorticoid receptor nuclear translocation in the AtT20 cell line,” Ann. NY Acad. Sci. 1148:536-541; Betanoff et al. (2011) “Selective glucocorticoid receptor (type II) antagonists prevent weight gain caused by olanzapine in rats” Eur. J. Pharmacol. 655(1-3): 117-120; and U.S. Patent Application Publication No. 2010/0135956.

Exemplary glucocorticoid receptor antagonists include, but are not limited to, those in the following classes of chemical compounds: octahydrophenanthrenes, spirocyclic dihydropyridines, triphenylmethanes and diaryl ethers, chromenes, dibenzyl anilines, dihydroisoquinolines, pyrimidinediones, azadecalins, and aryl pyrazolo azadecalins, as described by Clark, 2008. Some exemplary steroidal antagonists as described by Clark, 2008 include RU-486, RU-43044, 11-monoaryl and 11,21 bisaryl steroids (including 11β-substituted steroids), 10β-substituted steroids, 11β-aryl conjugates of mifepristone, and phosphorous-containing mifepristone analogs. Exemplary nonsteroidal antagonists include octahydrophenanthrenes, spirocyclic dihydropyridines, triphenylmethanes and diaryl ethers, chromenes, dibenzyl anilines, dihyrdroquinolines, pyrimidinediones, azadecalins, aryl pyrazolo azadecalins (including 8α-benzyl isoquinolones, N-substituted derivatives, bridgehead alcohol and ethers, bridgehead amines). Additional specific examples of GR antagonists include, but are not limited to the following specific antagonists: beclometasone, betamethasone, budesonide, ciclesonide, flunisolide, fluticasone, mifepristone, mometasone, triamcinolone, ORG-34517 (Merck), RU-43044, dexamethasone mesylate (DEX-Mes), dexamethasone oxetanone (DEX-Ox), deoxycorticosterone (DOC), CORT 0113083, and CORT 00112716.

In some embodiments of any embodiment of the combination therapy methods described herein, the other agent is any one or any combination of the GR down-regulator, such as those described in this section, or the derivatives thereof.

### Cancers for Treatment

Cancers discussed herein include, but are not limited to, adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, and glioblastoma), breast cancer, bronchial adenomasicarcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small lung cancer, non-small cell lung cancer, adenocarcinoma of the lung, and squamous carcinoma of the lung), lymphoid neoplasm (e.g., lymphoma), medulloblastoma, melanoma, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglimia), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), and Meigs' syndrome.

In some embodiments of any of the methods, the cancer is selected from the group consisting of lung cancer (e.g., NCSLC or SCLC), uterine cancer (e.g., leiomyosarcoma), kidney cancer, ovarian cancer, breast cancer, endometrial cancer, head & neck cancer, pancreatic cancer, and melanoma.

In some embodiments, the cancer is selected from the group consisting of breast cancer, lung cancer, and pancreatic cancer. In some embodiments, the cancer is triple negative breast cancer (TNBC). In some embodiments, the cancer is non-small cell lung cancer (NSCLC). In some embodiments, the cancer is pancreatic ductal adenocarcinoma (PDAC).

In some embodiments of any of the methods, the cancer is a solid tumor. In some embodiments, the solid tumor includes, but is not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Kaposi's sarcoma, soft tissue sarcoma, uterine sacronomasynovioma, mesothelioma. Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pincaloma, hemangioblastoma, acoustic neuroma, oligodendroglioma, menangioma, melanoma, neuroblastoma, and retinoblastoma.

In some embodiments of any of the methods, the cancer is breast cancer. In some embodiments, the breast cancer is early stage breast cancer, non-metastatic breast cancer, advanced breast cancer, stage IV breast cancer, locally advanced breast cancer, metastatic breast cancer, breast cancer in remission, breast cancer in an adjuvant setting, or breast cancer in a neoadjuvant setting. In some specific embodiments, the breast cancer is in a neoadjuvant setting. In some embodiments, there are provided methods of treating cancer at advanced stage(s).

In some embodiments of any of the methods, the cancer is a renal cell carcinoma (also called kidney cancer, renal adenocarcinoma, or hypernephroma). In some embodiments, the renal cell carcinoma is an adenocarcinoma. In some embodiments, the renal cell carcinoma is a clear cell renal cell carcinoma, papillary renal cell carcinoma (also called chromophilic renal cell carcinoma), chromophobe renal cell carcinoma, collecting duct renal cell carcinoma, granular renal cell carcinoma, mixed granular renal cell carcinoma, renal angiomyolipomas, or spindle renal cell carcinoma. In some embodiments, the renal cell carcinoma is associated with (1) von Hippel-Lindau (VHL) syndrome, (2) hereditary papillary renal carcinoma (HPRC), (3) familial renal oncocytoma (FRO) associated with Birt-Hogg-Dube syndrome (BHDS), or (4) hereditary renal carcinoma (HRC). There are provided methods of treating renal cell carcinoma at any of the four stages, I, II, III, or IV, according to the American Joint Committee on Cancer (AJCC) staging groups. In some embodiments, the renal cell carcinoma is stage IV renal cell carcinoma.

In some embodiments of any of the methods, the cancer is prostate cancer. In some embodiments, the prostate cancer is an adenocarcinoma. In some embodiments, the prostate cancer is a sarcoma, neuroendocrine tumor, small cell cancer, ductal cancer, or a lymphoma. There are provided methods of treating prostate cancer at any of the four stages, A, B, C, or D, according to the Jewett staging system. In some embodiments, the prostate cancer is stage A prostate cancer (The cancer cannot be felt during a rectal exam.). In some embodiments, the prostate cancer is stage B prostate cancer (The tumor involves more tissue within the prostate, it can be felt during a rectal exam, or it is found with a biopsy that is done because of a high PSA level.). In some embodiments, the prostate cancer is stage C prostate cancer (The cancer has spread outside the prostate to nearby tissues.). In some embodiments, the prostate cancer is stage D prostate cancer. In some embodiments, the prostate cancer may be androgen independent prostate cancer (AIPC). In some embodiments, the prostate cancer may be androgen dependent prostate cancer. In some embodiments, the prostate cancer may be refractory to hormone therapy. In some embodiments, the prostate cancer may be substantially refractory to hormone therapy.

In some embodiments of any of the methods, the cancer is lung cancer. In some embodiments, the cancer is lung cancer is a non-small cell lung cancer (NSCLC). Examples of NSCLC include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the NSCLC may be, according to TNM classifications, a stage T tumor (primary tumor), a stage N tumor (regional lymph nodes), or a stage M tumor (distant metastasis). In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the cancer is small cell lung cancer (SCLC; also called oat cell carcinoma). The small cell lung cancer may be limited-stage, extensive stage or recurrent small cell lung cancer.

In some embodiments of any of the methods, the cancer is brain cancer. In some embodiments, the brain cancer is glioma, brain stem glioma, cerebellar or cerebral astrocytoma (e.g., pilocytic astrocytoma, diffuse astrocytoma, or anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, or glioblastoma. In some embodiments, the brain cancer is glioblastoma (also called glioblastoma multiforme or grade 4 astrocytoma). In some embodiments, the glioblastoma is radiation-resistant. In some embodiments, the glioblastoma is radiation-sensitive. In some embodiments, the glioblastoma may be infratentorial. In some embodiments, the glioblastoma is supratentorial.

In some embodiments of any of the methods, the cancer is melanoma. In some embodiments, the melanoma is cutaneous melanoma. In some embodiments, the melanoma is metastatic melanoma. In some embodiments, the melanoma is metastatic malignant melanoma. In some embodiments, the melanoma is stage IV melanoma (e.g., stage IV cutaneous melanoma). In some embodiments, the metastatic melanoma is at stage M1a. In some embodiments, the metastatic melanoma is at stage M1b. In some embodiments, the metastatic melanoma is at stage M1c. In some embodiments, the individual has not received prior therapy (e.g., prior cytotoxic chemotherapy) for the melanoma (e.g., metastatic melanoma). In some embodiments, the melanoma comprises a mutation in BRAF. In some embodiments, the melanoma does not comprise a mutation in BRAF. In some embodiments, the melanoma is cutaneous melanoma. In some embodiments, the melanoma is melanoma of the skin. In some embodiments, the melanoma is superficial spreading melanoma. In some embodiments, the melanoma is nodular melanoma. In some embodiments, the melanoma is acral lentiginous melanoma. In some embodiments, the melanoma is lentigo maligna melanoma. In some embodiments, the melanoma is mucosal melanoma (e.g., mucosal melanoma in nose, mouth, throat, or genital area). In some embodiments, the melanoma is ocular melanoma. In some embodiments, the melanoma is uveal melanoma. In some embodiments, the melanoma is choroidal melanoma. Melanoma described herein may also be any of the following: cutaneous melanoma, extracutaneous melanoma, superficial spreading melanoma, malignant melanoma, nodular malignant melanoma, nodular melanoma, polypoid melanoma, acral lentiginous melanoma, lentiginous malignant melanoma, amelanotic melanoma, lentigo maligna melanoma, mucosal lentignous melanoma, mucosal melanoma, soft-tissue melanoma, ocular melanoma, desmoplastic melanoma, or metastatic malignant melanoma.

In some embodiments of any of the methods, the cancer is ovarian cancer. In some embodiments, the cancer is ovarian epithelial cancer. Exemplary ovarian epithelial cancer histological classifications include: serous cystomas (e.g., serous benign cystadenomas, serous cystadenomas with proliferating activity of the epithelial cells and nuclear abnormalities but with no infiltrative destructive growth, or serous cystadenocarcinomas), mucinous cystomas (e.g., mucinous benign cystadenomas, mucinous cystadenomas with proliferating activity of the epithelial cells and nuclear abnormalities but with no infiltrative destructive growth, or mucinous cystadenocarcinomas), endometrioid tumors (e.g., endometrioid benign cysts, endometrioid tumors with proliferating activity of the epithelial cells and nuclear abnormalities but with no infiltrative destructive growth, or endometrioid adenocarcinomas), clear cell (mesonephroid) tumors (e.g., begin clear cell tumors, clear cell tumors with proliferating activity of the epithelial cells and nuclear abnormalities but with no infiltrative destructive growth, or clear cell cystadenocarcinomas), unclassified tumors that cannot be allotted to one of the above groups, or other malignant tumors. In various embodiments, the ovarian epithelial cancer is stage I (e.g., stage IA, IB, or IC), stage II (e.g., stage IIA, IIB, or IIC), stage III (e.g., stage IIIA, IIIB, or IIIC), or stage IV.

In some embodiments, the cancer is an ovarian germ cell tumor. Exemplary histologic subtypes include dysgerminomas or other germ cell tumors (e.g., endodermal sinus tumors such as hepatoid or intestinal tumors, embryonal carcinomas, olyembryomas, choriocarcinomas, teratomas, or mixed form tumors). Exemplary teratomas are immature teratomas, mature teratomas, solid teratomas, and cystic teratomas (e.g., dermoid cysts such as mature cystic teratomas, and dermoid cysts with malignant transformation). Some teratomas are monodermal and highly specialized, such as struma ovari, carcinoid, struma ovarii and carcinoid, or others (e.g., malignant neuroectodermal and ependymomas). In some embodiments, the ovarian germ cell tumor is stage I (e.g., stage IA, IB, or IC), stage II (e.g., stage IIA, IIB, or IIC), stage III (e.g., stage IIIA, IIIB, or IIIC), or stage IV.

In some embodiments of any of the methods, the cancer is a pancreatic cancer. In some embodiments, the pancreatic cancer is exocrine pancreatic cancer or endocrine pancreatic cancer. The exocrine pancreatic cancer includes, but is not limited to, adenocarcinomas, acinar cell carcinomas, adenosquamous carcinomas, colloid carcinomas, undifferentiated carcinomas with osteoclast-like giant cells, hepatoid carcinomas, intraductal papillary-mucinous neoplasms, mucinous cystic neoplasms, pancreatoblastomas, serous cystadenomas, signet ring cell carcinomas, solid and pseuodpapillary tumors, pancreatic ductal carcinomas, and undifferentiated carcinomas. In some embodiments, the exocrine pancreatic cancer is pancreatic ductal carcinoma. The endocrine pancreatic cancer includes, but is not limited to, insulinomas and glucagonomas.

In some embodiments, the pancreatic cancer is early stage pancreatic cancer, non-metastatic pancreatic cancer, primary pancreatic cancer, advanced pancreatic cancer, locally advanced pancreatic cancer, metastatic pancreatic cancer, unresectable pancreatic cancer, pancreatic cancer in remission, or recurrent pancreatic cancer. In some embodiments, the pancreatic cancer is locally advanced pancreatic cancer, unresectable pancreatic cancer, or metastatic pancreatic ductal carcinoma. In some embodiments, the pancreatic cancer is resistant to the gemcitabine-based therapy. In some embodiments, the pancreatic cancer is refractory to the gemcitabine-based therapy. In some embodiments, the pancreatic cancer is resectable (i.e., tumors that are confined to a portion of the pancreas or has spread just beyond it that allows for complete surgical removal), or locally advanced (unresectable) (i.e., the localized tumors may be unresectable because of local vessel impingement or invasion by tumor). In some embodiments, the pancreatic cancer is, according to American Joint Committee on Cancer (ADGC) TNM classifications, a stage 0 tumor (the tumor is confined to the top layers of pancreatic duct cells and has not invaded deeper tissues, and it has not spread outside of the pancreas (e.g., pancreatic carcinoma in situ or pancreatic intraepithelial neoplasia III), a stage IA tumor (the tumor is confined to the pancreas and is less than 2 cm in size, and it has not spread to nearby lymph nodes or distinct sites), a stage IB tumor (the tumor is confined to the pancreas and is larger than 2 cm in size, and it has not spread to nearby lymph nodes or distant sites), a stage IIA tumor (the tumor is growing outside the pancreas but not into large blood vessels, and it has not spread to nearby lymph nodes or distant sites), stage IIB (the tumor is either confined to the pancreas or growing outside the pancreas but not into nearby large blood vessels or major nerves, and it has spread to nearby lymph nodes but not distant sites), stage III (the tumor is growing outside the pancreas into nearby large blood vessels or major nerves, and it may or may not have spread to nearby lymph nodes. It has not spread to distant sites) or stage IV tumor (the cancer has spread to distant sites).

The methods provided herein can be used to treat an individual (e.g., human) who has been diagnosed with pancreatic cancer and has progressed on a prior therapy (e.g., gemcitabine-based, erlotinib-based, or 5-fluorouracil-based therapy). In some embodiments, the individual is resistant to treatment of pancreatic cancer with gemcitabine-based therapy (e.g., gemcitabine monotherapy or gemcitabine combination therapy) and has progressed after treatment (e.g., the pancreatic cancer has been refractory). In some embodiments, the individual is initially responsive to treatment of pancreatic cancer with gemcitabine-based therapy (e.g., gemcitabine monotherapy or gemcitabine combination therapy) but has progressed after treatment. In some embodiments, the individual is non-responsive, less responsive or has stopped responding to treatment with a chemotherapeutic agent (e.g., gemcitabine). In some embodiments, the individual is human. In some embodiments, the individual is at least about any of 30, 35, 40, 45, 50, 55, 60, 65, 70, 75, 80, or 85 years old. In some embodiments, the individual has a family history of pancreatic cancer (e.g., at least 2 first-degree relatives affected with pancreatic cancer without accumulation of other cancers or familial diseases). In some embodiments, the individual has one or more hereditary pancreatic cancer syndromes, including, but not limited to, BRCA2 mutation, familial atypical multiple mole melanoma (FAMMM), peutz-jeghers syndrome, and hereditary pancreatitis. In some embodiments, the individual is a long-time smoker (e.g., more than 10, 15, or 20 years). In some embodiments, the patient has adult-onset diabetes. In some embodiments, the individual is a male. In some embodiments, the individual is a female. In some embodiments, the individual has early stage of pancreatic cancer, non-metastatic pancreatic cancer, primary pancreatic cancer, resected pancreatic cancer, advanced pancreatic cancer, locally advanced pancreatic cancer, metastatic pancreatic cancer, unresectable pancreatic cancer, pancreatic cancer in remission, or recurrent pancreatic cancer. In some embodiments, the individual has Stage 0, IA, IB, IIA, IIB, III, or IV pancreatic cancer according to AJCC (American Joint Commission on Cancer) TNM staging criteria. In some embodiments, the individual has ECOG/WHO/Zubrod score of 0 (asymptomatic), I (symptomatic but completely ambulatory), 2 (symptomatic, <50% in bed during the day), 3 (symptomatic, >50% in bed, but not bedbound), or 4 (bedbound). In some embodiments, the individual has a single lesion at presentation. In some embodiments, the individual has multiple lesions at presentation.

In some embodiments, the individual is a human who exhibits one or more symptoms associated with pancreatic cancer. In some embodiments, the individual is at an early stage of pancreatic cancer. In some embodiments, the individual is at an advanced stage of pancreatic cancer. In some embodiments, the individual has non-metastatic pancreatic cancer. In some embodiments, the individual has primary pancreatic cancer. In some of embodiments, the individual is genetically or otherwise predisposed (e.g., having a risk factor) to developing pancreatic cancer. These risk factors include, but are not limited to, age, sex, race, diet, history of previous pancreatic cancer, presence of hereditary pancreatic cancer syndrome (e.g., BRCA2 mutation, familial atypical multiple mole melanoma, Peutz-Jeghers Syndrome, hereditary pancreatitis), genetic (e.g., familial pancreatic cancer) considerations, and environmental exposure. In some embodiments, the individuals at risk for pancreatic cancer include, e.g., those having at least 2 first-degree relatives who have experienced pancreatic cancer without accumulation of other cancers or familial diseases, and those whose risk is determined by analysis of genetic or biochemical markers (e.g., BRCA2, p16, STK11/LKB1, or PRSS1 gene). In some embodiments, the individual is positive for SPARC expression (for example based on IHC standard). In some embodiments, the individual is negative for SPARC expression.

In some embodiments, the individual has a pancreatic cancer (such as metastatic cancer). In some embodiments, the individual has locally advanced unresectable pancreatic cancer. In some embodiments, the primary location of the pancreatic cancer is the head of the pancreas. In some embodiments, the primary location of the pancreatic cancer is the body of the pancreas. In some embodiments, the primary location of the pancreatic cancer is the tail of the pancreas. In some embodiments, the individual has metastasis in the liver. In some embodiments, the individual has pulmonary metastasis. In some embodiments, the individual has peritoneal carcinomatosis. In some embodiments, the individual has stage IV pancreatic cancer at the time of diagnosis of pancreatic cancer. In some embodiments, the individual has 3 or more metastatic sites. In some embodiments, the individual has more than 3 metastatic sites. In some embodiments, the individual has a serum CA19-9 level that is ≧59×ULN (Upper Limit of Normal). In some embodiments, the individual has Karnofsky performance status (KPS) of between 70 and 80. In some embodiments, the individual has adenocarcinoma of the pancreas.

Any of the methods provided herein may be used to treat a primary tumor. Any of the methods of treatment provided herein may also be used to treat a metastatic cancer (that is, cancer that has metastasized from the primary tumor). Any of the methods provided herein may be used to treat cancer at an advanced stage. Any of the methods provided herein may be used to treat cancer at locally advanced stage. Any of the methods provided herein may be used to treat early stage cancer. Any of the methods provided herein may be used to treat cancer in remission. In some of the embodiments of any of the methods provided herein, the cancer has reoccurred after remission. In some embodiments of any of the methods provided herein, the cancer is progressive cancer. Any of the methods provided herein may be used to treat cancer substantially refractory to hormone therapy. Any of the methods provided herein may be used to treat HER-2 positive cancer. Any of the methods provided herein may be used to treat HER-2 negative cancer. In some embodiments of any of the methods, the cancer is estrogen and progesterone positive. In some embodiments of any of the methods, the cancer is estrogen and progesterone negative.

Any of the methods provided herein may be practiced in an adjuvant setting. Any of the methods provided herein may be practiced in a neoadjuvant setting, i.e., the method may be carried out before the primary/definitive therapy. In some embodiments, any of the methods provided herein may be used to treat an individual who has previously been treated. Any of the methods provided herein may be used to treat an individual who has not previously been treated. Any of the methods provided herein may be used to treat an individual at risk for developing cancer, but has not been diagnosed with cancer. Any of the methods provided herein may be used as a first line therapy. Any of the methods provided herein may be used as a second line therapy.

In some embodiments of any the methods described herein, the cancer is early stage cancer, non-metastatic cancer, primary cancer, advanced cancer, locally advanced cancer, metastatic cancer, cancer in remission, or recurrent cancer. In some embodiments, the cancer is localized resectable, localized unresectable, or unresectable.

Any of the methods provided herein may be used to treat an individual (e.g., human) who has been diagnosed with or is suspected of having cancer. In some embodiments, the individual may be a human who exhibits one or more symptoms associated with cancer. In some embodiments, the individual may have advanced disease or a lesser extent of disease, such as low tumor burden. In some embodiments, the individual is at an early stage of a cancer. In some embodiments, the individual is at an advanced stage of cancer. In some of the embodiments of any of the methods of treatment provided herein, the individual may be a human who is genetically or otherwise predisposed (e.g., risk factor) to developing cancer who has or has not been diagnosed with cancer. In some embodiments, these risk factors include, but are not limited to, age, sex, race, diet, history of previous disease, presence of precursor disease, genetic (e.g., hereditary) considerations, and environmental exposure (e.g., cigarette, pipe, or cigar smoking, exposure to second-hand smoke, radon, arsenic, asbestos, chromates, chloromethyl ethers, nickel, polycyclic aromatic hydrocarbons, radon progeny, other agents, or air pollution)

In some embodiments of any of the methods described herein, an individual (e.g., human) who has been diagnosed with or is suspected of having cancer can be treated. In some embodiments, the individual is human. In some embodiments, the individual is at least about any of 35, 40, 45, 50, 55, 60, 65, 70, 75, 80, or 85 years old. In some embodiments, the individual is male. In some embodiments, the individual is a female. In some embodiments, the individual has any of the types of cancer described herein. In some embodiments, the individual has a single lesion at presentation. In some embodiments, the individual has multiple lesions at presentation. In some embodiments, the individual is resistant to treatment of cancer with other agents (such as a non-nanoparticle formulation of taxane, e.g., Taxol® or Taxotere®). In some embodiments, the individual is initially responsive to treatment of cancer with other agents (such as a non-nanoparticle formulation of taxane, e.g., Taxol® or Taxotere®) but has progressed after treatment.

In some embodiments, the individual is characterized by a high level of GR. In some embodiments, the individual is characterized by a high level of GR in the tumor. In some embodiments, the individual is characterized by high GR expression. In some embodiments, the individual is characterized by a high GR mRNA level. In some embodiments, the individual has a GR protein level more than about any of at least about any of 2, 3, 4, 4.5, 5, 5.5, 6, 7, 8, 9, 10, 11, 12, 13, 14, or more times the protein level of a GAPDH as determined in a Western blot assay. In some embodiments, the individual is characterized by a high level of GR activity. In some embodiments, the individual is characterized by a high level (such as expression or activity) of a GR-activated molecule. In some embodiments, the individual is characterized by a low level (such as expression or activity) of a GR-repressed molecule.

In some embodiments, the individual is characterized by a high GC (such as cortisol) level. In some embodiments, the individual is characterized by a high endogenous GC (such as cortisol) level. In some embodiments, the individual is characterized by high GC secretion (such as in the blood (including plasma and serum), urine, or saliva). In some embodiments, the individual is characterized by a high GC (such as cortisol) level determined in a GC blood test with the blood sample taken in the morning, wherein the high GC (such as cortisol) level is at least about any of 1.3, 1.5, 1.7, 2, 3, 4, 5, or more times that of a normal GC level in the blood in the general healthy population (for example, about 5-23 μg/dL of cortisol in adults and children). In some embodiments, the individual is characterized by a high free-GC level determined in a 24-hour GC urine test, wherein the high free-GC level is at least about any of 1.3, 1.5, 1.7, 2, 3, 4, 5, or more times that of a normal free GC level in the general healthy population (for example, less than about 100 μg/dL or cortisol in adults, about 5-55 μg/dL in teens, or 2-27 μg/dL in children). In some embodiments, the individual is characterized by high GC (such as cortisol) activity. In some embodiments, the individual is characterized by a high level of GC (such as cortisol) in the blood. In some embodiments, the individual is characterized by a high GC (such as cortisol) level associated with chronic stress, such as physical and psychological stress associated with the cancer, such as anxiety, depression, headache, pain, fatigue, insomnia, anorexia, nausea, malnutrition, or any combination thereof. In some embodiments, the individual is characterized by a high GC (such as cortisol) level correlated with an advanced stage of cancer, such as any of T2, T3, T4, N1, N2, N3 or M1 stage of cancer based on the TNM staging system. In some embodiments, the individual has a high tumor burden, such as a large tumor size and/or a large number of cancer cells in the tumor bed. In some embodiments, the individual has palpable lymph nodes, or has cancer cells spread to nearby lymph nodes. In some embodiments, the individual has distant tumor metastases. In some embodiments, the individual is characterized by a high level of GR in the tumor and a high level of GC (such as cortisol) in the blood.

### Modes of Administration

The dose of the taxane (such as paclitaxel) compositions and/or the dose of GR down-regulator administered to an individual (such as a human) according to a method described herein may vary with the particular composition, the mode of administration, and the type of cancer described, herein being treated. The dose of the taxane (such as paclitaxel) compositions and/or the dose of GR down-regulator administered to an individual (such as a human) may also be adjusted (such as reduced) based on an individual's symptoms (such as adverse reactions). In some embodiments, the dose or amount is effective to result in a response. In some embodiments, the dose or amount is effective to result in an objective response (such as a partial response or a complete response). In some embodiments, the dose of the taxane (such as paclitaxel) composition (and/or the dose of GR down-regulator) administered is sufficient to produce an overall response rate of more than about any of 20%, 25%, 30%, 35%, 40%, 45%, 50%, 55%, 60%, 64%, 65%, 70%, 75%, 80%, 85 or 90% among a population of individuals treated with the taxane (such as paclitaxel) composition and/or GR down-regulator. Responses of an individual to the treatment of the methods described herein can be determined using methods known in the field.

In some embodiments, the amount of the taxane (such as paclitaxel) composition and/or the amount of GR down-regulator are sufficient to prolong progression-free survival of the individual. In some embodiments, the amount of the composition (and/or the dose of GR down-regulator) is sufficient to prolong survival of the individual. In some embodiments, the amount of the composition (and/or the dose of GR down-regulator) is sufficient to improve quality of life of the individual. In some embodiments, the amount of the composition (and/or the dose of GR down-regulator) is sufficient to produce clinical benefit of more than about any of 50%, 60%, 70%, or 77% among a population of individuals treated with the taxane (such as pactitaxel) composition and/or GR down-regulator.

In some embodiments, the amount of the taxane (such as paclitaxel) composition, or GR down-regulator is an amount sufficient to decrease the size of a pancreatic tumor, decrease the number of pancreatic tumor cells, or decrease the growth rate of a pancreatic tumor by at least about any of 10%, 20%, 30%, 40%, 50%, 60%, 70%, 80%, 90%, 95% or 100% compared to the corresponding tumor size, number of pancreatic tumor cells, or tumor growth rate in the same individual prior to treatment or compared to the corresponding activity in other individuals not receiving the treatment. Methods that can be used to measure the magnitude of this effect are known in the field.

In some embodiments, the amount of the taxane (e.g., paclitaxel) in the composition (and/or GR down-regulator) is below the level that induces a toxicological effect (i.e., an effect above a clinically acceptable level of toxicity) or is at a level where a potential side effect can be controlled or tolerated when the composition (and/or GR down-regulator) is administered to the individual.

In some embodiments, the amount of the composition (and/or GR down-regulator) is close to a maximum tolerated dose (MTD) of the composition (and/or GR down-regulator) following the same dosing regimen. In some embodiments, the amount of the composition (and/or GR down-regulator) is more than about any of 80%, 90%, 95%, or 98% of the MID.

In some embodiments, the amount of a taxane (e.g., paclitaxel) in the composition is included in any of the following ranges: about 0.1 mg to about 500 mg, about 0.1 mg to about 2.5 mg, about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount (dose) of a taxane (e.g., paclitaxel) in the composition (e.g., a unit dosage form) is in the range of about 5 mg to about 500 mg, such as about 30 mg to about 300 mg or about 50 mg to about 200 mg. In some embodiments, the concentration of the taxane (e.g., paclitaxel) in the composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the taxane (e.g., paclitaxel) is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml. In some embodiments, the concentration of the taxane (e.g., paclitaxel) is no more than about any of 100 mg/ml, 90 mg/ml, 80 mg/ml, 70 mg/ml, 60 mg/ml, 50 mg/ml, 40 mg/ml, 30 mg/ml, 20 mg/ml, 10 mg/ml, or 5 mg/ml.

Exemplary amounts (doses) of a taxane (e.g., paclitaxel) in the taxane composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of a taxane (e.g., paclitaxel). In various embodiments, the composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² of a taxane (e.g., paclitaxel). In some embodiments, the amount of the taxane (e.g., paclitaxel) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the amount (dose) of a taxane (e.g., paclitaxel) in the composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 100 to about 200 mg/m², about 125 to about 150 mg/m², about 125 to about 175 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the amount (dose) of a taxane (e.g., paclitaxel) in the composition is included in any of the following ranges: about 10 mg/m² to about 400 mg/m², about 25 mg/m² to about 400 mg/m², about 50 mg/m² to about 400 mg/m², about 75 mg/m² to about 350 mg/m², about 75 mg/m² to about 300 mg/m², about 75 mg/m² to about 250 mg/m², about 75 mg/m² to about 200 mg/m² about 75 mg/m² to about 150 mg/m² about 75 mg/m² to about 125 mg/m², about 100 mg/m² to about 260 mg/m², about 100 mg/m² to about 250 mg/m², about 100 mg/m² to about 200 mg/m², or about 125 mg/m² to about 175 mg/m². In some embodiments, the amount (dose) of a taxane (e.g., paclitaxel) in the composition is about 5 to about 300 mg/m², about 100 to about 200 mg/m², about 100 to about 150 mg/m², about 50 to about 150 mg/m², about 75 to about 150 mg/m², about 75 to about 125 mg/m², or about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 260 mg/m², or about 300 mg/m².

In some embodiments of any of the above aspects, the amount (dose) of a taxane (e.g., paclitaxel) in the composition includes at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In various embodiments, the amount (dose) of a taxane (e.g., paclitaxel) in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of a taxane (e.g., paclitaxel).

Exemplary dosing frequencies for the administration of the taxane compositions include, but are not limited to, daily, every two days, every three days, every four days, every five days, every six days, weekly without break, weekly for three out of four weeks, once every three weeks, once every two weeks, or two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the taxane (e.g., paclitaxel) is administered over a period of at least ten days, wherein the interval between each administration is no more than about two days, and wherein the dose of the taxane (e.g., paclitaxel) at each administration is about 0.25 mg/m² to about 250 mg/m², about 0.25 mg/m² to about 150 mg/m², about 0.25 mg/² to about 75 mg/m², such as about 0.25 mg/m² to about 25 mg/m², about 25 mg/m² to about 50 mg/m², or about 50 mg/m² to about 100 mg/m².

The administration of the composition can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months.

In some embodiments, the dosage of a taxane (e.g., paclitaxel) in a taxane composition can be in the range of 5-400 mg/m² when given on a 3 week schedule, or 5-250 mg/m² (such as 75-200 mg/m², 100-200 mg/m², for example 125-175 mg/m²) when given on a weekly schedule. For example, the amount of a taxane (e.g., paclitaxel) is about 60 to about 300 mg/m² (e.g., about 100 mg/m², 125 mg/m², 150 mg/m², 175 mg/m², 200 mg/m², 225 mg/m², 250 mg/m², or 260 mg/m²) on a three week schedule. In some embodiments, the amount of a taxane (e.g., paclitaxel) is about 60 to about 300 mg/m² (e.g., about 100 mg/m², 125 mg/m², 150 mg/m², 175 mg/m², 200 mg/m², 225 mg/m², 250 mg/m², or 260 mg/m²) administered weekly. In some embodiments, the amount of a taxane (e.g., paclitaxel) is about 60 to about 300 mg/m² (e.g., about 100 mg/m², 125 mg/m², 150 mg/m², 175 mg/m^{t}, 200 mg/m², 225 mg/m², 250 mg/m², or 260 mg/m²) administered weekly for three out of a four week schedule.

Other exemplary dosing schedules for the administration of the taxane composition (e.g., paclitaxel/albumin nanoparticle composition) include, but are not limited to, 100 mg/m², weekly, without break; 75 mg/m² weekly, 3 out of four weeks; 100 mg/m², weekly, 3 out of 4 weeks; 125 mg/m², weekly, 3 out of 4 weeks; 150 mg/m², weekly, 3 out of 4 weeks; 175 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², without break; 20-150 m/m² twice a week; and 150-250 mg/m² twice a week, 50-70 mg/m² twice a week, 50-70 mg/m² three times a week, 30-70 mg/m² daily. The dosing frequency of the composition may be adjusted over the course of the treatment based on the judgment of the administering physician.

In some embodiments, the individual is treated for at least about any of one, two, three, four, five, six, seven, eight, nine, or ten treatment cycles.

The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes.

Other exemplary doses of the taxane (in some embodiments paclitaxel) in the taxane composition include, but are not limited to, out any of 50 mg/m², 60 mg/m², 75 mg/m², 80 mg/m², 90 mg/m², 100 mg/m², 120 mg/m², 140 mg/m², 150 mg/ m², 160 mg/m², 175 mg/m², 200 mg/m², 210 mg/m², 220 mg/m², 260 mg/m², and 300 mg/m². For example, the dosage of paclitaxel in a taxane composition can be in the range of about 100-400 mg/m² when given on a 3 week schedule, or about 50-250 mg/m² when given on a weekly schedule.

In some embodiments, the cancer is breast cancer (for example metastatic breast cancer), and the composition is administered at 260 mg/m2 once every three weeks.

In some embodiments, the cancer is pancreatic cancer (for example advanced pancreatic cancer, or adenocarcinoma of the pancreas), and the composition is administered at 125 mg/m2 weekly, three out of four weeks. In some embodiments, the cancer is pancreatic cancer (for example advanced pancreatic cancer), and the composition is administered at 125 mg/m2 weekly, three out of four weeks in combination with gemcitabine at 1000 mg/m2.

In some embodiments, the cancer is lung cancer (for example non-small cell lung cancer), and the composition is administered at 100 mg/m2 weekly. In some embodiments, the cancer is lung cancer (for example non-small cell lung cancer), and the composition is administered at 100 mg/m2 weekly, such as on Days 1, 8, 15 of each three weeks cycle, in combination with carboplatin at AUC=6 mg·min/mL once every three weeks, such as on Day 1 of each three weeks cycle.

The methods described herein in some embodiments comprise further administering another agent that down-regulates GR (also referred to as a “GR down-regulator.”) The GR down-regulator administered to an individual according to a method described herein may be in the range of about 0.5 mg/m² to about 5 mg/m², about 5 mg/m² to about 10 mg/m², about 10 mg/m² to about 15 mg/m², about 15 mg/m²to about 20 mg/m², about 20 mg/m² to about 25 mg/m², about 20 mg/m² to about 50 mg/m², about 25 mg/m² to about 50 mg/m², about 50 mg/m² to about 75 mg/m², about 50 mg/m² to about 100 mg/m², about 75 mg/m² to about 100 mg/m², about 100 mg/m² to about 125 mg/m², about 125 mg/m² to about 150 mg/m², about 150 mg/m² to about 175 mg/m², about 175 mg/m² to about 200 mg/m², about 200 mg/m² to about 225 mg/m², about 225 mg/m² to about 250 mg/m², about 250 mg/m² to about 300 mg/m², about 300 mg/m² to about 350 mg/m², about 350 mg/m² to about 400 mg/m², about 400 mg/m²to about 450 mg/m², about 450 mg/m² to about 500 mg/m², about 500 mg/m² to about 600 mg/m², about 600 mg/m² to about 700 mg/m², about 700 mg/m² to about 800 mg/m², about 800 mg/m²to about 900 mg/m², about 900 mg/m² to about 1000 mg/m², about 1000 mg/m² to about 1250 mg/m², or about 1250 mg/m² to about 1500 mg/mL. Other exemplary ranges of the GR down-regulator include, but are not limited to: about 5000 mg/m², about 100 mg/m² to about 2000 mg/m², about 200 to about 4000 mg/m², about 300 to about 3000 mg/m², about 400 to about 2000 mg/m², about 500 to about 1500 mg/m², about 500 mg/m² to about 2000 mg/m² about 750 to about 1500 mg/m², about 800 to about 1500 mg/m², about 900 to about 1400 mg/m², about 900 to about 1250 mg/m², about 1000 to about 1500 mg/m², about 800 mg/m², about 850 mg/m², about 900 mg/m², about 950 mg/m², about 1000 mg/m², about 1050 mg/m², about 1100 mg/m², about 1150 mg/m², about 1200 mg/m², about 1250 mg/m², about 1300 mg/m², about 1350 mg/m², about 1400 mg/m², about 1450 mg/m², 1500 mg/m², 1550 mg/m², 1600 mg/m², 1700 mg/m², 1800 mg/m², 1900 mg/m², or 2000 mg/m². GR down-regulator may be administered by intravenous (IV) infusion, e.g., over a period of about 10 to about 300 minutes, about 15 to about 180 minutes, about 20 to about 60 minutes, about 10 minutes, about 20 minutes, or about 30 minutes.

In some embodiments, the amount (dose) of the GR down-regulator includes at least about any of 0.1 mg/kg, 1 mg/kg, 10 mg/kg, 15 mg/kg, 30 mg/kg, 50 mg/kg, 100 mg/kg, 200 mg/kg, 300 mg/kg, 400 mg/kg, 500 mg/kg, or more. In various embodiments, the amount (dose) of the GR down-regulator includes less than about any of 500 mg/kg, 400 mg/kg, 300 mg/kg, 200 mg/kg, 100 mg/kg, 50 mg/kg, 30 mg/kg, 15 mg/kg, 10 mg/kg, 1 mg/kg, 0.1 mg/kg, or less. In some embodiments, the amount (dose) of the GR down-regulator includes at least any of about 0.1 mg/kg to about 1 mg/kg, about 1 mg/kg to about 10 mg/kg, about 10 mg/kg to about 50 mg/kg, about 50 mg/kg to about 100 mg/kg, about 100 mg/kg to about 500 mg/kg, about 10 mg/kg to about 100 mg/kg, or about 0.1 mg/kg to about 100 mg/kg.

Exemplary dosing frequencies for the administration of GR down-regulator include, but are not limited to, daily, every two days, every three days, every four days, every five days, every six days, weekly without break, weekly for three out of four weeks, once every three weeks, once every two weeks, or two out of three weeks. In some embodiments, GR down-regulator is administered about once every 2 weeks, once every 3 weeks, once every 4 weeks, once every 6 weeks, or once every 8 weeks. In some embodiments, the GR down-regulator is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week. In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the GR down-regulator is administered over a period of at least ten days, wherein the interval between each administration is no more than about two days, and wherein the dose of the GR down-regulator at each administration is about 0.25 mg/m² to about 1500 mg/m², about 10 mg/m² to about 1000 mg/m², about 25 mg/m² to about 750 mg/m², such as about 25 mg/m² to about 500 mg/m², about 25 mg/m² to about 250 mg/m², or about 25 mg/m² to about 100 mg/m².

Other exemplary amounts of GR down-regulator include, but are not limited to, any of the following ranges: about 0.5 mg to about 5 mg, about 5 mg to about 10 mg, about 10 mg to about 15 mg, about 15 mg to about 20 mg, about 20 mg to about 25 mg, about 20 mg to about 50 mg, about 25 mg to about 50 mg, about 50 mg to about 75 mg, about 50 mg to about 100 mg, about 75 mg to about 100 mg, about 100 mg to about 125 mg, about 125 mg to about 150 mg, about 150 mg to about 175 mg, about 175 mg to about 200 mg, about 200 mg to about 225 mg, about 225 mg to about 250 mg, about 250 mg to about 300 mg, about 300 mg to about 350 mg, about 350 mg to about 400 mg, about 400 mg to about 450 mg, about 450 mg to about 500 mg, about 500 mg to about 600 mg, about 600 mg to about 700 mg, about 700 mg to about 800 mg, about 800 mg to about 900 mg, about 900 mg to about 1000 mg, about 1000 mg to about 1250 mg, or about 1250 mg to about 1500 mg.

The administration of GR down-regulator can be extended over an extended period of time, such as from about a month up to about seven years. In some embodiments, GR down-regulator is administered over a period of at least about any of 1, 2, 3, 4, 5, 6, 7, 8, 9, 10, 11, 12, 18, 24, 30, 36, 48, 60, 72, or 84 months.

The composition comprising a taxane (such as paclitaxel) (also referred to as “taxane composition”) and GR down-regulator can be administered simultaneously (i.e., simultaneous administration) and/or sequentially (i.e., sequential administration).

In some embodiments, the taxane composition and the GR down-regulator are administered simultaneously. The term “simultaneous administration,” as used herein, means that the taxane composition and the other agent are administered with a time separation of no more than about 15 minute(s), such as no more than about any of 10, 5, or 1 minutes. When the drugs are administered simultaneously, the drug in the nanoparticles and the other agent may be contained in the same composition (e.g., a composition comprising both the nanoparticles and the other agent) or in separate compositions (e.g., the nanoparticles are contained in one composition and the other agent is contained in another composition).

In some embodiments, the taxane composition and the GR down-regulator are administered sequentially. The term “sequential administration” as used herein means that the drug in the taxane composition and the other agent are administered with a time separation of more than about 15 minutes, such as more than about any of 20, 30, 40, 50, 60 or more minutes. In some embodiments, the time separation is at least about any of 1, 2, 3, 4, 5, 6, 7, 8, 9, 10, 11, 12, 16, 20, 24 or more hours. Either the taxane composition or the other agent may be administered first. In some embodiments, the GR down-regulator is administered prior to the administration of the taxane composition. In some embodiments, the GR down-regulator is administered after the administration of the taxane composition. The taxane composition and the other agent are contained in separate compositions, which may be contained in the same or different packages.

In some embodiments, the administration of the taxane composition and the GR down-regulator are concurrent, i.e., the administration period of the taxane composition and that of the GR down-regulator overlap with each other. In some embodiments, the taxane composition is administered for at least one cycle (for example, at least any of 2, 3, or 4 cycles) prior to the administration of GR down-regulator. In some embodiments, the GR down-regulator is administered for at least any of one, two, three, or four weeks. In some embodiments, the administrations of the taxane composition and the GR down-regulator are initiated at about the same time (for example, within any one of 1, 2, 3, 4, 5, 6, or 7 days). In some embodiments, the administrations of the taxane composition and the GR down-regulator are terminated at about the same time (for example, within any one of 1, 2, 3, 4, 5, 6, or 7 days). In some embodiments, the administration of the GR down-regulator continues (for example for about any one of 1, 2, 3, 4, 5, 6, 7, 8, 9, 10, 11, or 12 months) after the termination of the administration of the taxane composition. In some embodiments, the administration of GR down-regulator is initiated after (for example after about any one of 1, 2, 3, 4, 5, 6, 7, 8, 9, 10, 11, or 12 months) the initiation of the administration of the taxane composition. In some embodiments, the administrations of the taxane composition and the GR down-regulator are initiated and terminated at about the same time. In some embodiments, the administrations of the taxane composition and the GR down-regulator are initiated at about the same time and the administration of the GR down-regulator continues (for example for about any one of 1, 2, 3, 4, 5, 6, 7, 8, 9, 10, 11, or 12 months) after the termination of the administration of the taxane composition. In some embodiments, the administration of the taxane composition and GR down-regulator stop at about the same time and the administration of GR down-regulator is initiated after (for example after about any one of 1, 2, 3, 4, 5, 6, 7, 8, 9, 10, 11, or 12 months) the initiation of the administration of the taxane composition.

In some embodiments, the method comprises more than one treatment cycle, wherein at least one of the treatment cycles comprises the administration of (a) an effective amount of a composition comprising nanoparticles comprising a taxane (such as paclitaxel) and albumin; and (b) an effective amount of a GR down-regulator. In some embodiments, the treatment cycle comprises no less than about (such as about) 21 days (e.g., 4 weeks). In some embodiments, the treatment cycle comprises less than about 21 days (for example weekly or daily). In some embodiments, the treatment cycle comprises about 28 days.

In some embodiments, the administration of the taxane composition and GR down-regulator are non-concurrent. For example, in some embodiments, the administration of the taxane composition is terminated before GR down-regulator is administered. In some embodiments, the administration of GR down-regulator is terminated before the taxane composition is administered. The time period between these two non-concurrent administrations can range from about two to eight weeks, such as about four weeks.

The dosing frequency of the drug-containing taxane composition and GR down-regulator may be adjusted over the course of the treatment, based on the judgment of the administering physician. When administered separately, the drug-containing taxane composition and GR down-regulator can be administered at different dosing frequency or intervals. For example, the drug-containing taxane composition can be administered weekly, while GR down-regulator can be administered more or less frequently. In some embodiments, sustained continuous release formulation of the drug-containing nanoparticle and/or GR down-regulator may be used. Various formulations and devices for achieving sustained release are known in the art. Exemplary dosing frequencies are further provided herein.

The taxane composition and GR down-regulator can be administered using the same route of administration or different routes of administration. Exemplary administration routes are further provided herein. In some embodiments (for both simultaneous and sequential administrations), the taxane (such as paclitaxel) in the taxane composition and GR down-regulator are administered at a predetermined ratio. For example, in some embodiments, the ratio by weight of the taxane (such as paclitaxel) in the taxane composition and the GR down-regulator is about 1 to 1. In some embodiments, the weight ratio may be between about 0.001 to about 1 and about 1000 to about 1, or between about 0.01 to about 1 and 100 to about 1. In some embodiments, the ratio by weight of the taxane (such as paclitaxel) in the taxane composition and GR down-regulator is less than about any of 100:1, 50:1, 30:1, 10:1, 9:1, 8:1, 7:1, 6:1, 5:1, 4:1, 3:1, 2:1, and 1:1 In some embodiments, the ratio by weight of the taxane (such as paclitaxel) in the taxane composition and the GR down-regulator is more than about any of 1:1, 2:1, 3:1, 4:1, 5:1, 6:1, 7:1, 8:1, 9:1, 30:1, 50:1, 100:1. Other ratios are contemplated.

The doses required for the taxane (such as paclitaxel) and/or GR down-regulator may be lower than what is normally required when each agent is administered alone. Thus, in some embodiments, a subtherapeutic amount of the drug in the taxane composition and/or GR down-regulator are administered. “Subtherapeutic amount” or “subtherapeutic level” refer to an amount that is less than therapeutic amount, that is, less than the amount normally used when the drug in the taxane composition and/or GR down-regulator are administered alone. The reduction may be reflected in terms of the amount administered at a given administration and/or the amount administered over a given period of time (reduced frequency).

In some embodiments, enough GR down-regulator is administered so as to allow reduction of the normal dose of the drug in the taxane composition required to affect the same degree of treatment by at least about any of 5%, 10%, 20%, 30%, 50%, 60%, 70%, 80%, 90%, or more. In some embodiments, enough taxane (such as paclitaxel) in the taxane composition is administered so as to allow reduction of the normal dose of GR down-regulator required to effect the same degree of treatment by at least about any of 5%, 10%, 20%, 30%, 50%, 60%, 70%, 80%, 90%, or more.

In some embodiments, the dose of both the taxane (such as paclitaxel) in the taxane composition and GR down-regulator are reduced as compared to the corresponding normal dose of each when administered alone. in some embodiments, both the taxane (such as paclitaxel) in the taxane composition and GR down-regulator are administered at a subtherapeutic, i.e., reduced, level. In some embodiments, the dose of the taxane composition and/or GR down-regulator is substantially less than the established maximum toxic dose (MTD). For example, the dose of the taxane composition and/or GR down-regulator is less than about 50%, 40%, 30%, 20%, or 10% of the MTD.

In some embodiments, the dose of taxane (such as paclitaxel) and/or the dose of GR down-regulator is higher than what is normally required when each agent is administered alone. For example, in some embodiments, the dose of the taxane composition and/or GR down-regulator is substantially higher than the established maximum toxic dose (MTD). For example, the dose of the taxane composition and/or GR down-regulator is more than about 50%, 40%, 30%, 20%, or 10% of the MTD of the agent when administered alone.

As will be understood by those of ordinary skill in the art, the appropriate doses of GR down-regulator will be approximately those already employed in clinical therapies wherein the GR down-regulator is administered alone or in combination with other agents. Variation in dosage will likely occur depending on the condition being treated. As described above, in some embodiments, GR down-regulator may be administered at a reduced level.

The taxane compositions and/or GR down-regulator can be administered to an individual (such as human) via various routes, including, for example, parenteral, intravenous, intraventricular, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition and/or GR down-regulator may be used. In some embodiments, the composition (and/or GR down-regulator) is administered intravenously. In some embodiments, the composition (and/or GR down-regulator) is administered intraportally. In some embodiments, the composition (and/or GR down-regulator) is administered intraarterially. In some embodiments, the composition (and/or GR down-regulator) is administered intraperitoneally. In some embodiments, the composition (and/or GR down-regulator) is administered intrathecally. In some embodiments, the composition (and/or GR down-regulator) is administered through a ported catheter to spinal fluid. In some embodiments, the composition (and/or GR down-regulator) is administered intraventricularly. In some embodiments, the composition (and/or GR down-regulator) is administered systemically. In some embodiments, the composition (and/or GR down-regulator) is administered by infusion. In some embodiments, the composition (and/or GR down-regulator) is administered by infusion through implanted pump. In some embodiments, the composition (and/or GR down-regulator) is administered by a ventricular catheter. In some embodiments, the composition (and/or GR down-regulator) is administered through a port or portacath. In some embodiments, the port or portacath is inserted into a vein (such as jugular vein, subclavian vein, or superior vena cava).

In some embodiments, there is provided a method of treating pancreatic cancer (e.g., metastatic pancreatic adenocarcinoma) in an individual comprising administering to the individual (a) an effective amount of a composition comprising nanoparticles comprising a taxane (such as paclitaxel) and a carrier protein; and (b) an effective amount of GR down-regulator, wherein the dose of taxane (such as paclitaxel) in the taxane composition is between about 50 mg/m² to about 400 mg/m² (including for example about 100 mg/m² to about 300 mg/m², about 100 mg/m² to about 200 mg/m², or about 100 mg/m² to about 150 mg/m², or about 100 mg/m², or about 125 mg/m², or about 150 mg/m²) and the dose of GR down-regulator is about 500 mg/m² to about 2000 mg/m² (for example, about 750 mg/m² to about 1500 mg/m², about 800 mg/m² to about 1200 mg/m², about 750 mg/m², about 1000 mg/m², about 1250 mg/m², or about 1500 mg/m²). In some embodiments, the taxane composition is administered weekly for three weeks of four weeks or weekly. In some embodiments, GR down-regulator is administered weekly for three weeks of four weeks or weekly.

A combination of the administration configurations described herein can be used. A method described herein may be performed alone or in conjunction with an additional therapy, such as chemotherapy, radiation therapy, surgery, hormone therapy, gene therapy, immunotherapy, chemoimmunotherapy, cryotherapy, ultrasound therapy, liver transplantation, local ablative therapy, radiofrequency ablation therapy, photodynamic therapy, and the like.

### Taxane Compositions

The compositions described herein comprise taxanes, including for example paclitaxel, docetaxel, and ortataxel. In some embodiments, the taxane composition comprises nanoparticles comprising a taxane. In some embodiments, the taxane composition comprises nanoparticles comprising a taxane and a carrier protein. In some embodiments, the taxane composition can be used without premedication.

In some embodiments, the nanoparticle composition is substantially free as free) of surfactants (such as Cremophor®, Tween 80, or other organic solvents used for the administration of taxanes). In some embodiments, the nanoparticle composition contains less than about any one of 20%, 15%, 10%, 7.5%, 5%, 2.5%, or 1% organic solvent.

In some embodiments, the composition comprises nanoparticles comprising a taxane. In some embodiments, the composition comprises nanoparticle comprising a taxane, and a polymer (such as a block-copolymer). Exemplary nanoparticle compositions are described in WO2001087345A1, incorporated herein by reference.

In some embodiments, the composition comprises a taxane and a carrier protein. The term “proteins” refers to polypeptides or polymers of amino acids of any length (including full length or fragments), which may be linear or branched, comprise modified amino acids, and/or be interrupted by non-amino acids. The term also encompasses an amino acid polymer that has been modified naturally or by intervention; for example, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The proteins described herein may be naturally occurring, i.e., obtained or derived from a natural source (such as blood), or synthesized (such as chemically synthesized or by synthesized by recombinant DNA techniques). Examples of suitable carrier proteins include proteins normally found in blood or plasma, which include, but are not limited to, albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the carrier protein is non-blood protein, such as casein, α-lactalbumin, and β-lactoglobulin. The carrier proteins may either be natural in origin or synthetically prepared.

In some embodiments, the composition is a composition comprising nanoparticles comprising a taxane (such as paclitaxel) and albumin, as described below in more detail.

### Albumin-Based Nanoparticle Compositions

The taxane compositions described herein in some embodiments comprise nanoparticles comprising (in various embodiments consisting essentially of) taxane (e.g., paclitaxel) and an albumin (such as human serum albumin). Nanoparticles of poorly water soluble drugs (such as taxane) have been disclosed in, for example, U.S. Pat. Nos. 5,916,596; 6,506,405; 6,749,868, and 6,537,579 and also in U.S. Pat, Pub. Nos. 2005/0004002, 2006/0263434, and 2007/0082838; PCT Patent Application WO08/137148, each of which is incorporated by reference in their entirety.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprise taxane (e.g., paclitaxel) coated with an albumin (e.g., human serum albumin). In some embodiments, the composition comprises taxane (e.g., paclitaxel) in both nanoparticle and non-nanoparticle forms, wherein at least about any one of 50%, 60%, 70%, 80%, 90%, 95%, or 99% of taxane (e.g., paclitaxel) in the composition are in nanoparticle form. In some embodiments, taxane (e.g., paclitaxel) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of taxane (e.g., paclitaxel) that is substantially free of polymeric materials (such as polymeric matrix).

In some embodiments, the composition comprises albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of albumin (such as human serum albumin) and taxane (e.g., paclitaxel) in the nanoparticle composition is about 18:1 or less, such as about 15:1 or less, for example about 10:1 or less. In some embodiments, the weight ratio of albumin (such as human serum albumin) and taxane (e.g., paclitaxel) in the composition falls within the range of any one of about 1:1 to about 18:1, about 2:1 to about 15:1, about 3:1 to about 13:1, about 4:1 to about 12:1, or about 5:1 to about 10:1. In some embodiments, the weight ratio of albumin and taxane (e.g., paclitaxel) in the nanoparticle portion of the composition is about any one of 1:2, 1:3, 1:4, 1:5, 1:10, 1:15, or less. In some embodiments, the weight ratio of the albumin (such as human serum albumin) and taxane (e.g., paclitaxel) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1 about 1:1 to about 5:1, about 1:1 to about 4:1, about 1:1 to about 3:1, about 1:1 to about 2:1, or about 1:1 to about 1:1.

In some embodiments, the nanoparticle composition comprises one or more of the above characteristics.

In some embodiments of any of the methods described herein, the composition comprising nanoparticles comprising a taxane (e.g. paclitaxel) and an albumin (such as human serum albumin). In some embodiments, the taxane (e.g., paclitaxel) in the nanoparticles is coated with the albumin. In some embodiments, the average particle size of the nanoparticles in the composition is no greater than about 200 nm (such as less than about 200 nm, for example about 130 nm). In some embodiments, the composition comprises Nab-paclitaxel (Abraxane®). In some embodiments, the composition is the Nab-paclitaxel (Abraxane®).

In some embodiments, the taxane is selected from a group consisting of paclitaxel, docetaxel, ortataxel, and protaxel. In some embodiments the taxane is docetaxel. In some embodiments, the taxane is paclitaxel.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises human serum albumin. In some embodiments, the albumin (e.g., HSA) is recombinant albumin. Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977)) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context).

Human serum albumin (HSA) has multiple hydrophobic binding sites (a total of eight for fatty acids, an endogenous ligand of HSA) and binds a diverse set of taxanes, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vonim, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Paclitaxel has been shown to bind HSA (see, e.g., Paal et al., Eur. J. Biochem., 268(7), 2187-91 (200a)).

The albumin (such as human serum albumin) in the composition generally serves as a carrier for taxane (e.g., paclitaxel), i.e., the albumin in the composition makes taxane (e.g., paclitaxel) more readily suspendable in an aqueous medium or helps maintain the suspension as compared to compositions not comprising an albumin. This can avoid the use of toxic solvents (or surfactants) for solubilizing taxane (e.g., paclitaxel), and thereby can reduce one or more side effects of administration of taxane (e.g., paclitaxel) into an individual (such as a human). Thus, in some embodiments, the composition described herein is substantially free (such as free) of surfactants, such as Cremophor (including Cremophor EL® (BASF)). In some embodiments, the nanoparticle composition is substantially free (such as free) of surfactants. A composition is “substantially free of Cremophor” or “substantially free of surfactant” if the amount of Cremophor or surfactant in the composition is not sufficient to cause one or more side effect(s) in an individual when the nanoparticle composition is administered to the individual. In some embodiments, the nanoparticle composition contains less than about any one of 20%, 15%, 10%, 7.5%, 5%, 2.5%, or 1% organic solvent or surfactant.

The amount of albumin in the composition described herein will vary depending on other components in the composition. In some embodiments, the composition comprises an albumin in an amount that is sufficient to stabilize taxane (e.g., paclitaxel) in an aqueous suspension, for example, in the form of a stable colloidal suspension (such as a stable suspension of nanoparticles). In some embodiments, the albumin is in an amount that reduces the sedimentation rate of taxane (e.g., paclitaxel) in an aqueous medium. For particle-containing compositions, the amount of the albumin also depends on the size and density of nanoparticles of taxane (e.g., paclitaxel).

Taxane (e.g., paclitaxel) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin is present in an amount that is sufficient to stabilize taxane (e.g., paclitaxel) in an aqueous suspension at a certain concentration. For example, the concentration of taxane (e.g., paclitaxel) in the composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of taxane (e.g., paclitaxel) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of albumin. In some embodiments, the composition, in liquid form, comprises about 0.5% to about 5% (w/v) of albumin.

In some embodiments, the weight ratio of albumin, e.g., albumin, to taxane (e.g., paclitaxel) the nanoparticle composition is such that a sufficient amount of taxane (e.g., paclitaxel) binds to, or is transported by, the cell. While the weight ratio of albumin to taxane (e.g., paclitaxel) will have to be optimized for different albumin and taxane (e.g., paclitaxel) combinations, generally the weight ratio of albumin, e.g., albumin, to taxane (e.g., paclitaxel) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to taxane (e.g., paclitaxel) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human serum albumin) and taxane (e.g., paclitaxel) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, or about 1:1 to about 1:1.

In some embodiments, the albumin allows the composition to be administered to an individual (such as human) without significant side effects. In some embodiments, the albumin (such as human serum albumin) is in an amount that is effective to reduce one or more side effects of administration of taxane (e.g., paclitaxel) to a human. The term “reducing one or more side effects of administration” refers to reduction, alleviation, elimination, or avoidance of one or more undesirable effects caused by taxane (e.g., paclitaxel), as well as side effects caused by delivery vehicles (such as solvents that render taxane (e.g., paclitaxel) suitable for injection) used to deliver taxane (e.g., paclitaxel). In some embodiments, the one or more side effects are adverse side effects (AEs). In some embodiments, the one or more side effects are serious adverse side effects (SAEs). Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with taxane (e.g., paclitaxel) can be reduced.

In some embodiments, the nanoparticle compositions described herein comprises nanoparticles comprising a taxane (such as paclitaxel) and an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) and an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) and an albumin (such as human albumin or human serum albumin), wherein the nanoparticles have an average diameter of about 130 nm. In some embodiments, the nanoparticle compositions described herein comprises nanoparticles comprising paclitaxel and human albumin (such as human serum albumin), wherein the nanoparticles have an average diameter of about 130 nm.

In some embodiments, the nanoparticle compositions described herein comprises nanoparticles comprising a taxane (such as paclitaxel) and an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) and an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) and an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel and human albumin (such as human serum albumin), wherein the nanoparticles have an average diameter of about 130 nm, wherein the weight ratio of albumin and the taxane in the composition is about 9:1.

In some embodiments, the nanoparticle compositions described herein comprises nanoparticles comprising a taxane (such as paclitaxel) coated with an albumin (such as human albumin or human serum albumin). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) coated with an albumin (such as human albumin or human serum albumin), wherein the nanoparticles have an average diameter of about 130 nm. In some embodiments, the nanoparticle compositions described herein comprises nanoparticles comprising paclitaxel coated with human albumin (such as human serum albumin), wherein the nanoparticles have an average diameter of about 130 nm.

In some embodiments, the nanoparticle compositions described herein comprises nanoparticles comprising a taxane (such as paclitaxel) coated with an albumin (such as human albumin or human serum albumin), wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel coated with human albumin (such as human serum albumin), wherein the nanoparticles have an average diameter of about 130 nm, wherein the weight ratio of albumin and the taxane in the composition is about 9:1.

In some embodiments, the nanoparticle compositions described herein comprises nanoparticles comprising a taxane (such as paclitaxel) stabilized by an albumin (such as human albumin or human serum albumin). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) stabilized by an albumin (such as human albumin or human serum albumin), wherein the nanoparticles have an average diameter of about 130 nm. In some embodiments, the nanoparticle compositions described herein comprises nanoparticles comprising paclitaxel stabilized by human albumin (such as human serum albumin), wherein the nanoparticles have an average diameter of about 130 nm.

In some embodiments, the nanoparticle compositions described herein comprises nanoparticles comprising a taxane (such as paclitaxel) stabilized by an albumin (such as human albumin or human serum albumin), wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel stabilized by human albumin (such as human serum albumin), wherein the nanoparticles have an average diameter of about 130 nm, wherein the weight ratio of albumin and the taxane in the composition is about 9:1.

In some embodiments, the nanoparticle composition comprises Abraxane® (Nab-paclitaxel). In some embodiments, the nanoparticle composition is Abraxane® (Nab-paclitaxel). Abraxane® is a formulation of paclitaxel stabilized by human albumin USP, which can be dispersed in directly injectable physiological solution. The weight ratio of human albumin and paclitaxel is about 9:1. When dispersed in a suitable aqueous medium such as 0.9% sodium chloride injection or 5% dextrose injection, Abraxane® forms a stable colloidal suspension of paclitaxel. The mean particle size of the nanoparticles in the colloidal suspension is about 130 nanometers. Since HSA is freely soluble in water, Abraxane® can be reconstituted in a wide range of concentrations ranging from dilute (0.1 mg/ml paclitaxel) to concentrated (20 mg/ml paclitaxel), including for example about 2 to about 8 mg/ml, about 5 mg/ml.

Methods of making nanoparticle compositions are known in the art. For example, nanoparticles containing taxane (e.g., paclitaxel) and albumin (such as human serum albumin) can be prepared under conditions of high shear forces (e.g., sonication, high pressure homogenization, or the like). These methods are disclosed in, for example, U.S. Pat. Nos. 5,916,596; 6,506,405; 6,749,868, and 6,537,579 and also in U.S. Pat. Pub. No. 2005/0004002, 2007/0082838, 2006/0263434and PCT Application WO08/137148 and WO08/109163.

Briefly, taxane (e.g., paclitaxel) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (e.g., with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1.

### Other Components in the Nanoparticle Compositions

The nanoparticles described, herein can be present in a composition that includes other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the nanoparticle composition (see, e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Kits, Medicines, and Compositions

The invention also provides kits, medicines, compositions, and unit dosage forms for use in any of the methods described herein.

Kits of the invention include one or more containers comprising taxane (e.g., paclitaxel) compositions (or unit dosage forms and/or articles of manufacture) and optionally a GR down-regulator, and in some embodiments, optionally further comprise instructions for use in accordance with any of the methods described herein including methods for treating, assessing responsiveness, monitoring, identifying individuals, and selecting patients for treatment comprising a) a taxane (such as nanoparticles comprising a taxane and albumin) and optionally b) a GR down-regulator based upon levels of a GR and/or GC (such as cortisol) in a sample. The kit may comprise a description on selection of an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a) a composition comprising a taxane (such as a composition comprising nanoparticles comprising taxane (e.g., paclitaxel) and an albumin (such as human serum albumin)), b) an effective amount of a GR down-regulator, and c) instructions for screening a GR receptor in a sample. The taxane composition and the GR down-regulator can be present in separate containers or in a single container. For example, the kit may comprise one distinct composition or two or more compositions wherein one composition comprises taxane and one composition comprises GR down-regulator.

The kits of the invention are in suitable packaging. Suitable packaging include, but is not limited to, vials, bottles, jars, flexible packaging (e.g., Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions may also comprise instructions relating to the use of the taxane (e.g., paclitaxel) nanoparticle compositions (and optionally the GR down-regulator) generally include information as to dosage, dosing schedule, and route of administration for the intended treatment.

In some embodiments, the taxane (e.g., paclitaxel) composition and/or the GR down-regulator is administered intravenously.

The containers may be unit doses, bulk packages (e.g., multi-dose packages) or sub-unit doses. For example, kits may be provided that contain sufficient dosages of taxane (e.g., paclitaxel) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more.

Kits may also include multiple unit doses of taxane (e.g., paclitaxel) and pharmaceutical compositions and instructions for use and packaged in quantities sufficient for storage and use in pharmacies, for example, hospital pharmacies and compounding pharmacies.

Also provided are medicines, compositions, and unit dosage forms useful for the methods described herein. In some embodiments, there is provided a medicine (or composition or a unit dosage form) for use in treating cancer, optionally in conjunction with the GR down-regulator, comprising a taxane (for example nanoparticles comprising taxane (e.g., paclitaxel) and an albumin (such as human serum albumin)). In some embodiments, there is provided a medicine (or composition or a unit dosage form) for use in treating cancer, comprising a taxane (for example nanoparticles comprising taxane (e.g., paclitaxel) and an albumin (such as human serum albumin)) and the GR down-regulator.

The examples below are intended to be purely exemplary of the invention and should therefore not be considered to limit the invention in any way. The following examples and detailed description are offered by way of illustration and not by way of limitation.

### Exemplary Embodiments

Embodiment 1. In some embodiments, there is provided a method of treating an individual having a cancer, wherein the individual is characterized by a high level of glucocorticoid receptor (GR), comprising administering to the individual an effective amount of a composition comprising a taxane.

Embodiment 2. In some embodiments, there is provided a method of treating an individual having a cancer, wherein the individual is characterized by a high level of glucocorticoid (GC), comprising administering to the individual an effective amount of a composition comprising a taxane.

Embodiment 3. In some further embodiments of embodiment 2, the cancer is further characterized by a high level of GR.

Embodiment 4. In some embodiments, there is provided a method of treating an individual having a cancer, comprising administering to the individual: a) an effective amount of a composition comprising a taxane; and b) an effective amount of another agent that down-regulates GR.

Embodiment 5. In some further embodiments of embodiment 4, the individual is characterized by a high level of GR.

Embodiment 6. In some further embodiments of embodiment 4 or embodiment 5, the individual is characterized by a high level of GC.

Embodiment 7. In some further embodiments of any one of embodiments 1, 3, and 5-6, a high level of GR is used as a basis for selecting the individual for treatment.

Embodiment 8. In some further embodiments of embodiment 7, the method further comprises determining the level of GR in the individual.

Embodiment 9. In some further embodiments of any one of embodiments 2-3 and 6-7, a high level of GC is used as a basis for selecting the individual for treatment.

Embodiment 10. In some further embodiments of embodiment 9, the method further comprises determining the level of GC in the individual.

Embodiment 11. In some further embodiments of any one of embodiments 1, 3, and 5-10, the individual is characterized by a high level of GR expression.

Embodiment 12. In some further embodiments of any one of embodiments 1, 3, and 5-11, the individual is characterized by a high level of GR activity.

Embodiment 13. In some further embodiments of embodiment 12, the high level of GR activity is determined by measuring the expression or activity of a GR responsive molecule.

Embodiment 14. In some further embodiments of any one of embodiments 2-3 and 6-13, the individual is characterized by a high level of GC secretion.

Embodiment 15. In some further embodiments of any one of embodiments 2-3 and 6-14, the individual is characterized by high level of GC activity.

Embodiment 16. In some further embodiments of any one of embodiments 4-15, the other agent is an inhibitor of GR expression.

Embodiment 17. In some further embodiments of any one of embodiments 4-15, the other agent is an inhibitor of GR activity.

Embodiment 18. In some further embodiments of embodiment 17, the other agent is a GR antagonist.

Embodiment 19. In some further embodiments of embodiment 17, the other agent is a modulator of a GR responsive molecule.

Embodiment 20. In some further embodiments of embodiment 13 or embodiment 19, the GR responsive molecule is selected from the group consisting of SGK1, MKP1, MCL1, SAP30, DUSP1, SMARCA2, PTGDS, TNFRSF9, SFN, LAPTM5, GPSM2, SORT1, DPT, NRP1, ACSL5, BIRC3, NNMT, IGFBP6, PLXNC1, SLC46A3, C14orf139, PIAS1, SERPINF1, ERBB2, PECAM1, LBH, ST3GAL5, IL1R1, BIN1, WIPF1, TFP1, FN1, FAM134A, NRIP1, RAC2, SPP1, PHF15, BTN3A2, SESN1, MAP3K5, DPYSL2, SEMA4D, STOM, MAOA, SLUG, SERPINE1, RGS2, KRT7, MME, JAK2, CEBPD, IL6, LIF, and TNFRSF11B.

Embodiment 21. In some further embodiments of any one of embodiments 1-20, the cancer is selected from the group consisting of breast cancer, lung cancer, and pancreatic cancer.

Embodiment 22. In some further embodiments of embodiment 21, the cancer is pancreatic cancer.

Embodiment 23. In some further embodiments of any one of embodiments 1-22, the cancer is advanced cancer.

Embodiment 24. In some further embodiments of any one of embodiments 4-23, the composition comprising the taxane and the other agent are administered simultaneously.

Embodiment 25. In some further embodiments of any one of embodiments 4-23, the composition comprising the taxane and the other agent are administered sequentially.

Embodiment 26. In some further embodiments of any one of embodiments 1-25, the composition comprising the taxane is administered intravenously.

Embodiment 27. In some further embodiments of any one of embodiments 1-26, the taxane is paclitaxel.

Embodiment 28. In some further embodiments of any one of embodiments 1-27, the composition comprises nanoparticles comprising the taxane.

Embodiment 29. In some further embodiments of embodiment 28, the composition comprises nanoparticles comprising the taxane and an albumin.

Embodiment 30. In some further embodiments of embodiment 29, the nanoparticles in the composition comprise the taxane coated with the albumin.

Embodiment 31. In some further embodiments of any one of embodiments 28-30, the nanoparticles in the composition have an average diameter of no greater than about 200 nm.

Embodiment 32. In some further embodiments of any one of embodiments 29-31, the albumin is human albumin.

Embodiment 33. In some further embodiments of any one of embodiments 1-32, the individual is human.

### EXAMPLE

The examples below are intended to be purely exemplary of the invention and should therefore not be considered to limit the invention in any way. The following examples and detailed description are offered by way of illustration and not by way of limitation.

### Example 1

### Dexamethasone Inhibits Paclitaxel-Induced Apoptosis in MDA-MB-231 Cells

This example demonstrates apoptosis measurements of MDA-MB-231 cells (a breast cancer cell line) following administration of paclitaxel (PTX) with and without pretreatment of 100 nM dexamethasone (DEA). Measurements were made over a span of about 34.5 hours following administration of PTX or PTX/DEX.

MDA-MB-231 cells were cultured using Eagle's minimum essential medium (EMEM) with 10% fetal bovine serum (FBS). FBS was charcoal-filtered to remove endogenous steroids. Cells were plated at 5000 cell per well and treated 24 hours after plating. PTX doses of 3000, 1000, 333.3, 111.1, 37.0, 12.3, 4.1, 1.4, 0.5, and 0 nM were evaluated. MDA-MB-231 cell samples treated with DEX received 100 nM DEX 30 minutes prior to administration of PTX or a vehicle control. The vehicle control for DEX was 0.1% ethanol, final concentration after addition. The vehicle control for PTX was 0.1% DMSO, final concentration after addition. Additionally, during PTX and/or DEX drug dosing, a caspase-3/7 activation reagent was added to the cell samples, for example, Caspase-3/7 reagent (Essen BioScience, Ann Arbor, Mich.; Catalog No. 4440). Specifically, the CELLPLAYER™ Kinetic Caspase-3/7 activation assay reagents (Essen BioScience, Ann Arbor, Mich.) were used to detect caspase-3/7 activation.

At time points following PTX administration (e.g., roughly every 3 hours) the activation of the caspase-3/7 reagent was measured with, for example, an INCUCYTE™ FLR (Essen BioScience, Ann Arbor, Mich.). Activation of caspase-3/7 is indicative of an apoptotic and/or an apoptosing cell. All concentrations of PTX, both with and without DEX, were evaluated in triplicate.

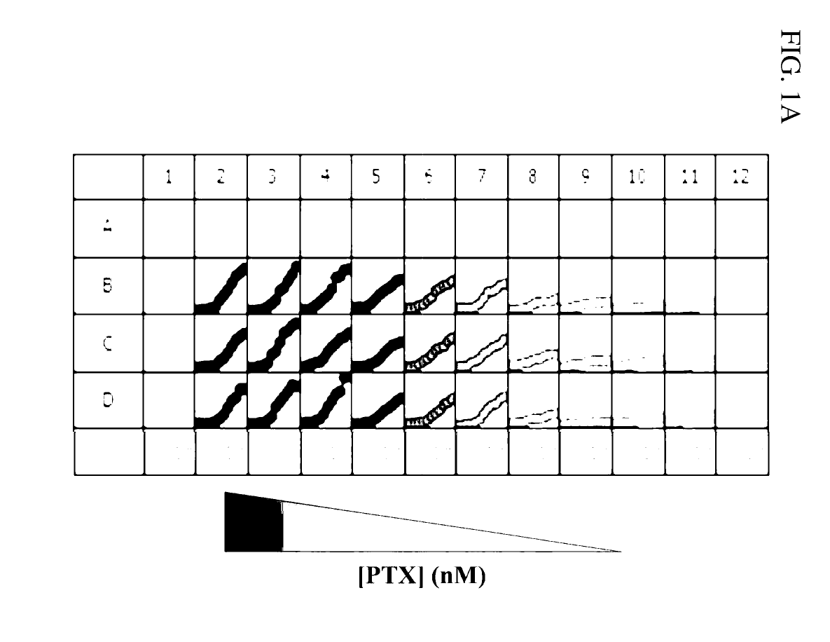

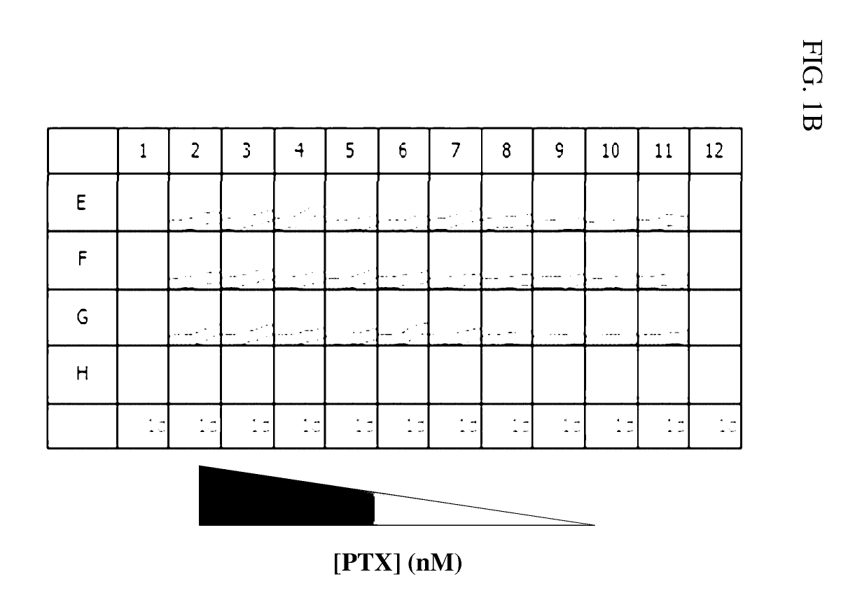

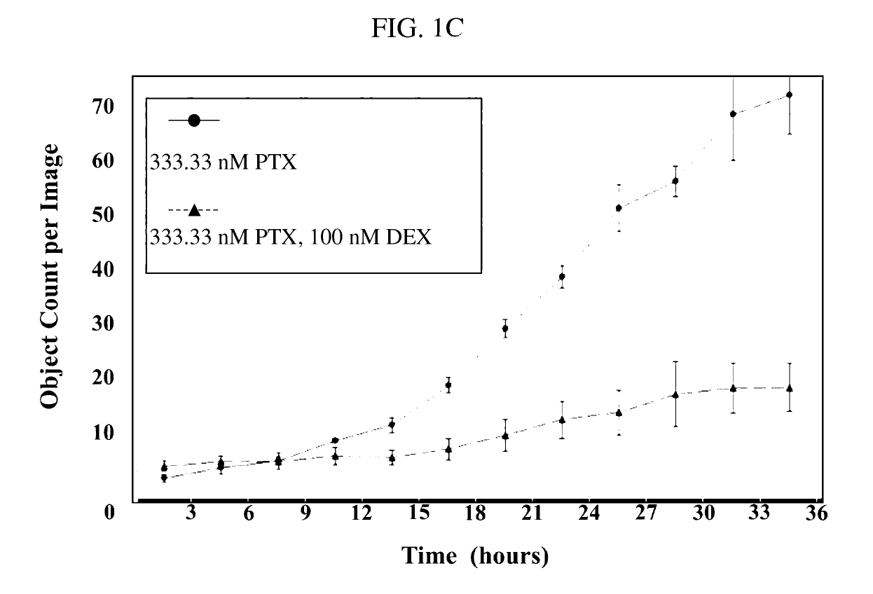

As observed in FIG. 1A, the level of apoptosis (as measured by level of caspase-3/7 activation) in MDA-MB-231 cells following administration of PTX increases with time (each square represents measurements over roughly 34.5 hours following administration of PTX). As observed in FIG. 1A, higher concentrations of PTX induced increased levels of apoptosis during the studied time span. As observed in FIG. 1B, preincubation with 100 nM DEX reduced the level of PTX-induced apoptosis. FIG. 1C illustrates a comparison of the level of apoptosis over time for cell samples administered 333.33 nM PTX and cell samples administered 333.33 nM PTX and 100 nM DEX. As shown, DEX inhibits PTX-induced apoptosis of MDA-MB-231 cells (FIG. 1C).

### Example 2

### Dexamethasone Inhibits Paclitaxel-Induced Apoptosis in H1755 Cells

This example demonstrates apoptosis measurements of H1755 cells (a non-small cell lung cancer (NSCLC) cell line) following treatment with PTX with and without DEX pretreatment. Furthermore, this example details the determination of the EC₅₀ of DEX in H1755 cells.

H1755 cells were cultured using EMEM with 10% FBS. FBS was charcoal-filtered to remove endogenous steroids. Cells were plated at 5000 cell per well and treated 24 hours after plating. H1755 cell samples treated with DEX received DEX 30 minutes prior to administration of PTX or a vehicle control. Vehicle controls are the same as those discussed in Example 1. Additionally, during PTX and/or DEX drug dosing, a caspase-3/7 activation reagent was added to the cell samples, for example, Caspase-3/7 reagent (Essen BioScience, Ann Arbor, Mich; Catalog No. 4440). Specifically, the CELLPLAYER™ Kinetic Caspase-3/7 activation assay reagents (Essen BioScience, Ann Arbor, Mich.) were used to detect caspase-3/7 activation.

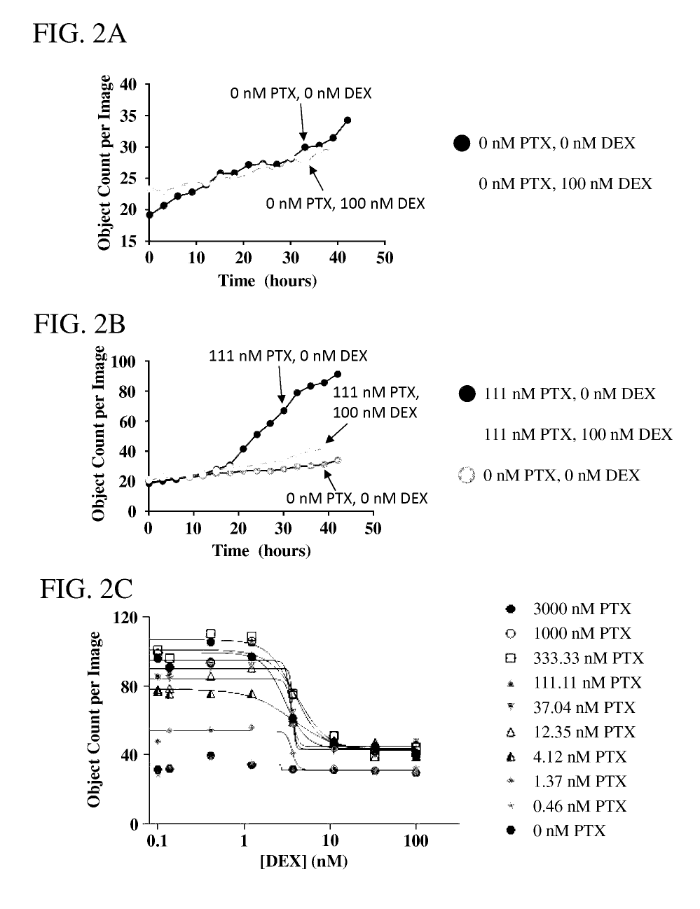

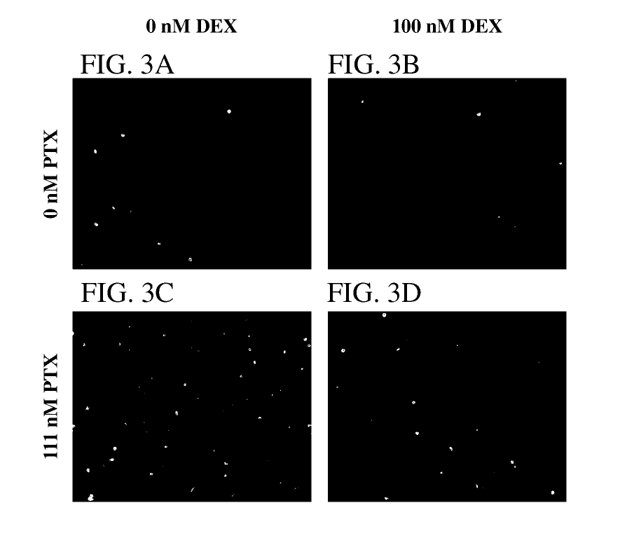

At time points following PTX administration the activation of the caspase-3/7 reagent was measured with, for example, an INCUCYTE™ FLR (Essen BioScience, Ann Arbor, Mich.). Images were acquired every 3 hours. As observed in FIG. 2A, a similar level of apoptosis (as measured by level of caspase-3/7 activation) in H1755 cells was observed following administration of either 0 nM DEX or 100 nM DEX. As observed in FIG. 2B, H1755 cells exhibit a marked increase in apoptosis following administration of 111 nM PTX. The increase in PTX-induced was antagonized by co-administration of DEX (FIG. 2B). As illustrated in FIG. 2B, the level of apoptosis for H1755 cell samples treated with 100 nM PTX and 100 nM DEX were similar to H1755 cell samples that did not received treatment with PTX or DEX. Representative images of caspase-3/7 activation 40 hours after drug administration are shown in FIGS. 3A-3D. Apoptotic or apoptosing H1755 cells (caspase-3/7 positive cells) are indicated in white, as measured by caspase-3/7 activation (FIGS. 3A-3D).

To determine the EC₅₀ of DEX in H1755 cells, a series of PTX concentrations were evaluated for caspase-3/7 activation across a series of DEX concentrations. The EC₅₀ of DEX in H1755 cells was determined to be 4 nM (FIG. 2C).

### Example 3

### Correlation of the Presence of Glucocorticoid Receptor (GR) and the Observation of DEX Antagonism of PTX-Induced Apoptosis

This example demonstrates the correlation of the presence of (glucocorticoid receptor) GR in a cell and the observation of DEX antagonism of PTX-induced apoptosis for NSCLC cell lines (A549, H1755, and H522 cells), triple-negative breast cancer (TNBC) cell lines (MM231, CAL120, BT549), and pancreatic ductal adenocarcinoma (PDAC) cell lines (HS766t, Panc03.27, AsPC1). Furthermore, this example demonstrates that the calculated inhibition of apoptosis (based on an apoptosis index) for NSCLC, TNBC, and PDAC cell lines is predictive of DEX antagonism of PTX-induced apoptosis.

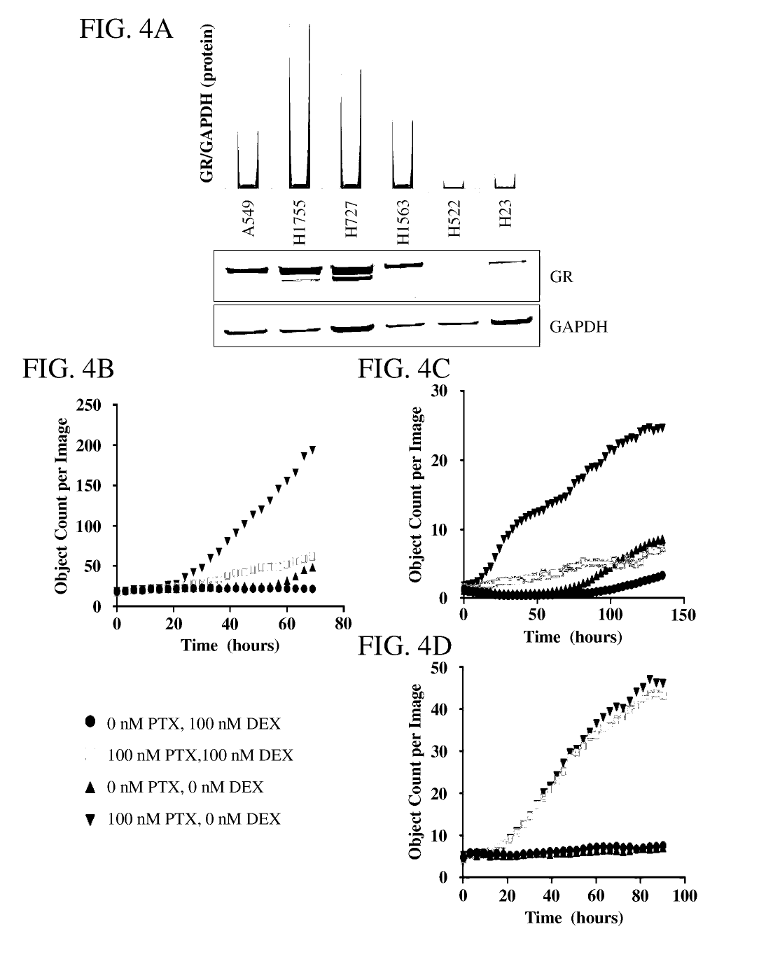

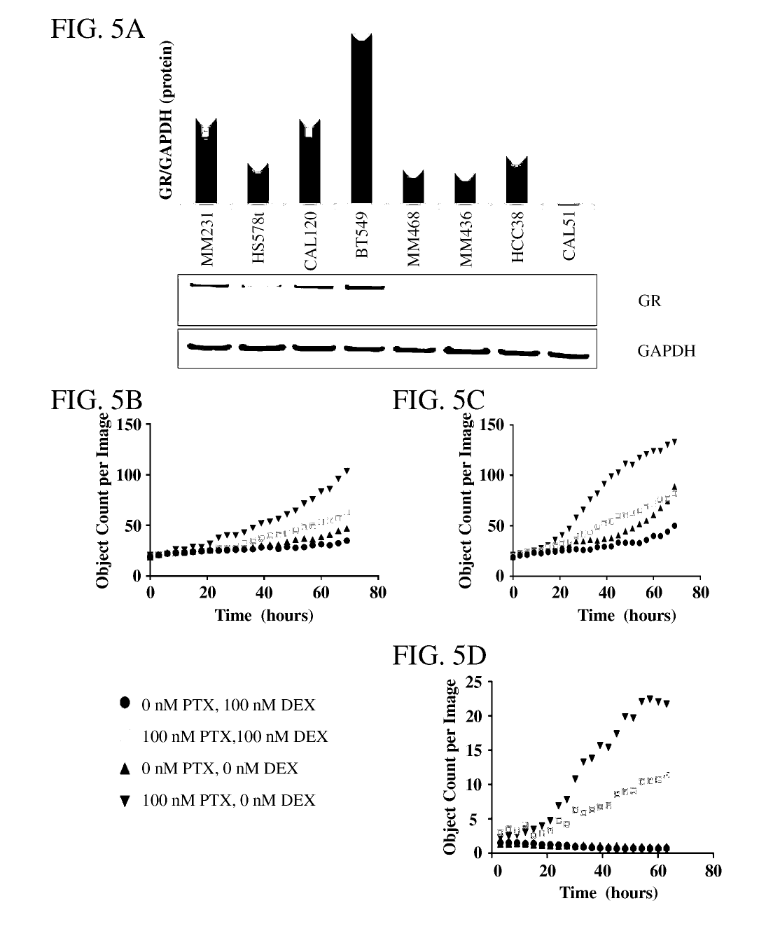

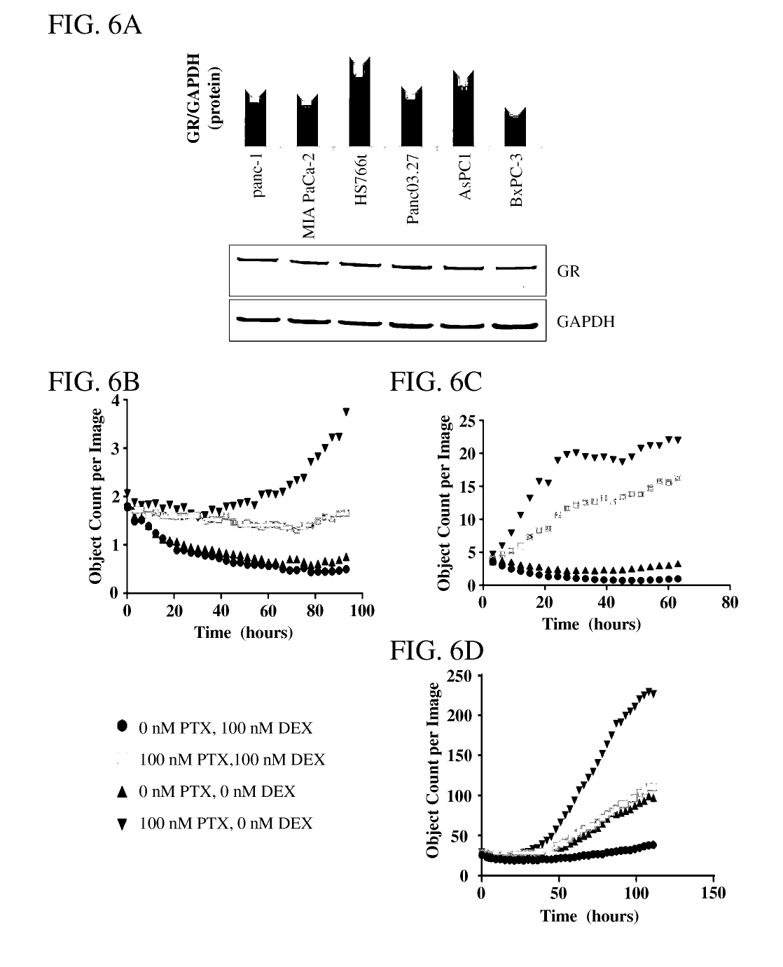

The amount of GR in six NSCLC cell lines (A549, H1755, H727, H1563, H522, and H23), 8 TNBC cell lines (MM231, HS578t, CAL120, BT549, MM468, MM436, HCC38, CAL51), and 6 PDAC cell lines (panc-1, MIA PaCa-2, HS766t, Panc03.27, AsPC1, BxPC-3) was determined by Western blot. The amount of GR in each cell line (as measured by GR/GAPDH) is illustrated for the above stated NSCLC, TNBC, and PDAC cell lines in FIG. 4A, FIG. 5A, and FIG. 6A, respectively.

With regard to NSCLC cell lines, the caspase-3/7 activation assay was performed (as described in Examples 1 and 2) for A549, H1755, and H522 cell lines. DEX antagonism of PTX-induced apoptosis was observed in A549 and H1755 cell lines (FIGS. 4B, 4C). A549 and H1755 cell lines both exhibited high levels of GR (FIG. 4A). DEX antagonism of PTX-induced apoptosis was not observed, in H522 cell line (FIG. 4D). H522 cell line exhibited a minimal level of GR (FIG. 4A).

With regard to TNBC cell lines, the caspase-3/7 activation was performed (as described in Examples 1 and 2) for MM231, CAL120, and BT549 cell lines. DEX antagonism of PTX-induced apoptosis was observed in MM231, CAL120, and BT549 cell lines (FIGS. 5B-5D). MM231, CAL120, and BT549 cell lines exhibited high levels of GR (FIG. 5A). DEX antagonism of PTX-induced apoptosis was not observed in CAL51 cell line (data not shown; CAL51 cell line exhibited a minimal level of GR (FIG. 5A)).

With regard to PDAC cell lines, the caspase-3/7 activation assay was performed (as described in Examples 1 and 2) for HS766t, Pac03.27, and AsPC1 cell lines. DEX antagonism of PTX-induced apoptosis was observed in HS766t, Pac03.27, and AsPC1 cell lines (FIGS. 6B-6D), HS766t, Pac03.27, and AsPC1 cell lines exhibited high levels of GR (FIG. 6A).

As illustrated in FIGS. 4A-6D, NSCLC, TNBC, and PDAC cell lines with high levels of GR exhibit a striking decrease in PTX-induced apoptosis when pretreated with DEX. Furthermore, GR expression was required for DEX sensitivity (e.g., DEX had no effect on H522 and H23 cells (data not shown for H23 cells); both cell lines exhibit a low level of GR expression).

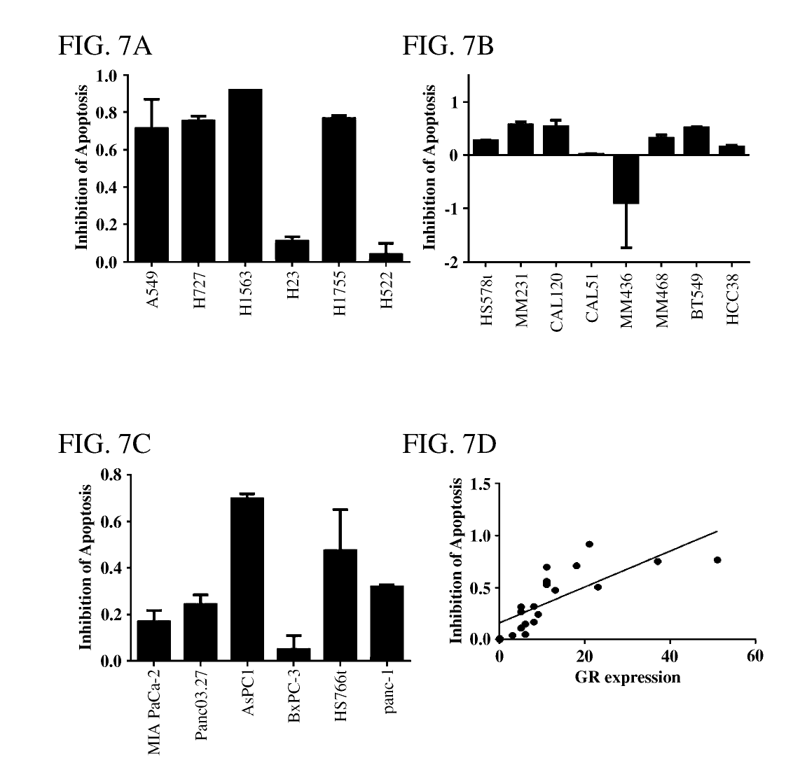

To compare DEX responses across NSCLC, TNBC, and PDAC cell lines, an apoptosis inhibition index was calculated using data measured for each cell line 66 hours after administration of PTX and/or DEX. The apoptosis inhibition index was calculated as a degree of inhibition of PTX-driven apoptosis by DEX: apoptosis inhibition index=1−[({DEX and PTX treatment}−{DEX treatment})/({PTX treatment}−{DEX treatment})]. Alternate methods of calculating an apoptosis inhibition index are conceived, for example, apoptosis inhibition index=(PTX_{AUC}−DEX_{AUC})/PTX_{AUC}. FIGS. 7A-7C show the calculated apoptosis index (as reported by inhibition of apoptosis) for each cell line. Visually, it is observed that most cell lines studied are classified as DEX-sensitive (i.e., DEX mediates inhibition of PTX-induced apoptosis; FIGS. 7A-7C). Apoptosis inhibition index values greater than about 15% are indicative of a DEX-sensitive cell line.

A comparative plot of inhibition of apoptosis versus GR expression of all cell lines studied illustrates a correlation between the level of GR expression and the degree of DEX-induced antagonism of PTX-induced apoptosis (FIG. 7D). The linear model resulted in an equation of: y=0.01.729x+0.1632, with a p-value of 0.0001, and the deviation from zero is significant (FIG. 7D). Thus, there is a quantitative association of the cytoprotective effect of DEX for PTX-induced apoptosis with GR expression. Furthermore, for the studied cell lines, it was observed that a GR expression level above about 5 can be predictive of a DEX-sensitive cell line.

### Example 4

### PTX-Induced Markers of Apoptosis are Down-Regulated by DEX

This example demonstrates DEX-mediated inhibition of key stress response pathways involved with antagonizing PTX-induced apoptosis signals.

H1755 and H522 cells were cultured using RPMI media that was charcoal-filtered media to remove steroids. Cell samples received one of the following treatments: vehicle control (0.1% EtOH for DEX vehicle control or 0.1% DMSO for PTX vehicle control), 100 nM DEX, 100 nM PTX, or 100 nM DEX and 100 nM PTX. At 1 hour, 4 hours, or 24 hours after drug administration, cell samples were harvested and mRNA levels of MKP-1 and SGK1 were measured via quantitative real-time polymerase chain reaction (qRT-PGR). Western blot analysis of GR, phosphorylated JNK1 (P-JNK1; phosphorylation at Thr 183 and Tyr 185), JNK1, phosphorylated c-Jun (P c-Jun; phosphorylation at Ser 73 or Ser 63), c-Jun, phosphorylated MKP1 (pMKP1; phosphorylated at Ser 359), SG1, phosphorylated MCL-1 (pMCL-1; phosphorylated at Ser 64), MCL-1, phosphorylated BCL2 (pBC1,2; phosphorylated at Ser 70), BCL2, BCLXL, cleaved caspase-3, and GAPDH (for purposes of normalization) was performed following treatment with either a vehicle control (0.1% EtOH for DEX vehicle control or 0.1% DMSO for PTX vehicle control), 100 nM DEX, 100 nM PTX, or 100 nM DEX and 100 nM PTX.

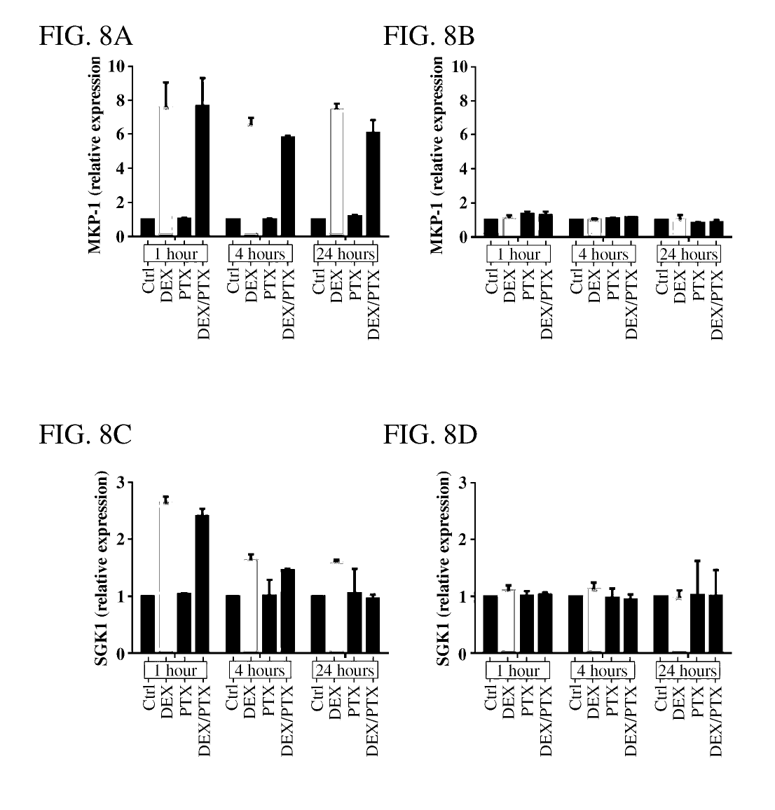

As reported in Example 3, H1755 cells have a high level of GR expression (GR positive) and H522 cells have a low level of GR expression (GR negative; FIG. 4A). mRNA measurements showed that DEX up-regulates expression of MKP-1 and SGK1 in H1755 cells, but not in H522 cells at all time points studied (FIGS. 8A-8D). DEX up-regulated MKP-1 (at a maximum of 8-fold) and SGK1 (at a maximum of 2.5-fold) in a GR-dependent manner. Furthermore, MKP-1 expression was sustained over the 24 hour period (FIG. 8A) and expression of SGK1 decreased over time following an initial increase at the 1 hour time point (FIG. 8C).

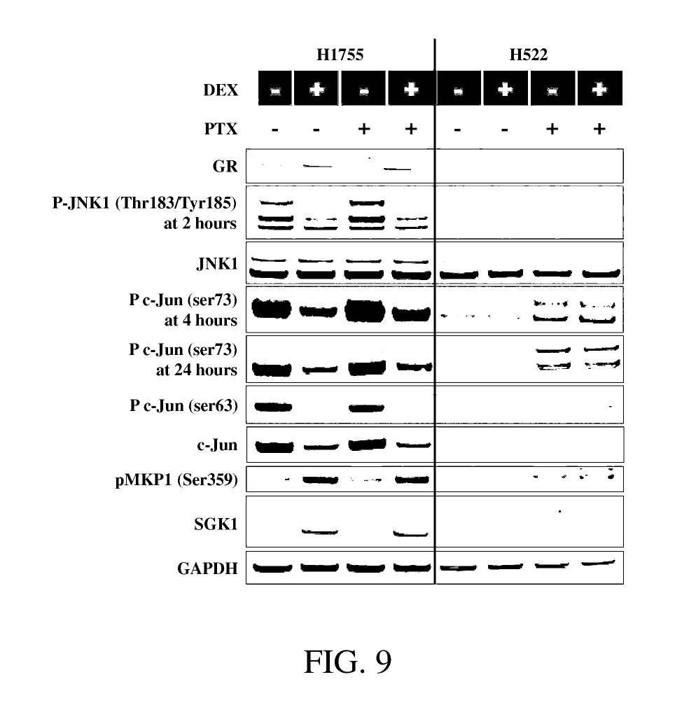

In addition to measuring MKP-1 and SGK1 mRNA expression, a series of proteins (and their phosphorylation status) involved in stress pathway signaling were evaluated via Western blot to measure changes following treatment with DEX, PTX, or DEX and PTX. As shown in FIG. 9. JNK1 was measured after 2 hours of treatment, P c-Jun, c-Jun, pMPK1, and SGK1 were measured after 4 hours of treatment, and other measurements were after 24 hours of treatment. As shown in FIG. 9, there was a high level of P-JNK1 and P c-Jun in H1755 cells. In contrast, in H522 cells, P-JNK1 levels were low, although expression level of JNK1 was comparable in H1755 and H522 cells (FIG. 9). Following PTX treatment, phosphorylation of c-Jun on Ser 73 was increased in both cell lines (FIG. 9). DEX inhibited PTX-induced phosphorylation of c-Jun (Ser 73) in H1755 cells but not H522 cells (FIG. 9). Furthermore, induction of pMKP-1 and SGK1 correlated with dephosphorylation of P-JNK1 and P c-Jun in H1755 cells (FIG. 9).

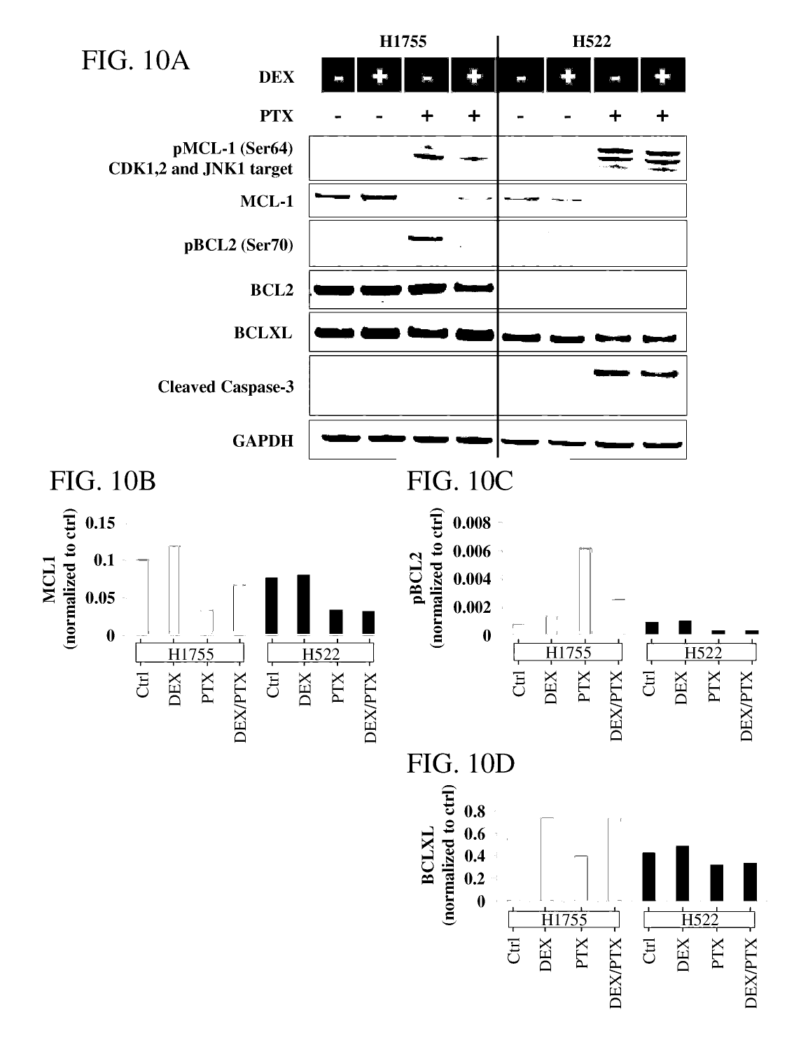

Furthermore, pro-survival markers, MCL1, BCL2, and BCLXL, were measured by Western blot to evaluate changes following treatment with DEX, PTX, or DEX and PTX. MCL1, BCL2, and BCLXL inhibit apoptosis by blocking activation of caspases, such as, caspase-3. As shown in FIGS. 10A-10D, measurements were after 24 hours of treatment. PTX up-regulated phosphorylation of MCL1 while decreasing total expression of MCL1 (FIG. 10A). This trend is partially reversed in H1755 cells, but not H522 cells (FIG. 10A). PTX also up-regulated phosphorylation of BCL2 in H1755 cells (FIG. 10A). Treatment with PTX and DEX decreased the phosphorylation of BLC2 (FIG. 10A). Phosphorylation of BLC2 is known to inhibit the anti-apoptotic function of this protein. PTX down-regulated BCLXL expression in H1755 cells, but not H522 cells (FIG. 10A). Multiple studies have shown that the anti-apoptotic effects of DEX are mediated by pro-survival protein BCLXL. See Herr Apoptosis 2007 and Gruver-Yates Cells 2013. As shown in FIG. 10A, treatment with PTX ultimately increases expression of cleaved caspase-3, a signal of apoptosis, in both H1755 cells and H522 cells. Co-administration of PTA and DEX abolished the presence of cleaved caspase-3 in H1755 cells, but not in H522 cells (FIG. 10A). Additionally, co-administration with DEX and PTA promotes pro-survival signals in H1755 cells, but not in H522 cells (FIGS. 10B-10C).

### Example 5

### MAP Tau is Up-Regulated by DEX in H1755 Cells

This example demonstrates DEX-induced expression of MAP TAU in GR positive cells.

H1755 and H522 cells were analyzed by Western blot at 4 and 24 hours following administration of a vehicle control (0.1% EtOH for DEX vehicle control or 0.1% DMSO for PTX vehicle control), 100 nM DEX, 100 nM PTX, or 100 nM DEX and 100 nM PTX.

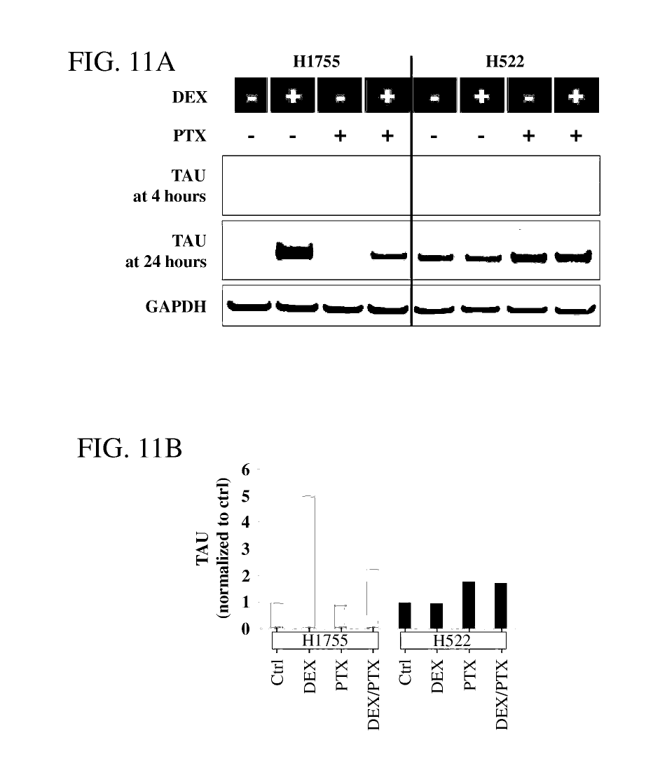

As shown in FIG. 11A, H1755 cells, a GR positive cell line, exhibited DEX-induced upregulation (5-fold increase) of MAP TAU at 24 hours after drug administration. A 2-fold increase was observed in H1755 cells treated with DEX and PTX (FIG. 11B). DEX-induced upregulation of MAP TAU was not observed in H522 cells, a GR negative cell line, at 4 or 24 hours following administration of DEX (FIG. 11A)

### Example 6

### shRNA Knockdown of GR Reduces Anti-Apoptotic Effect of DEX

This example demonstrates the role of GR in DEX-mediated inhibition of PTX-induced apoptosis.

H1755 cells (a GR positive cell line) and H1755 cells following knockdown of GR expression were studied following DEX and/or PTX administration. shRNA was used to knock down GR expression in H1755 cells. Doxorubicin (DOX) was used to induce shRNA knockdown of GR. Cells were treated with 0 nM PTX and 0 nM DEX. 0 nM PTX and 100 nM DEX, 100 nM PTX and 100 nM DEX, or 100 nM PTX and 100 nM DEX. Vehicle controls were 0.1% EtOH for the DEX vehicle control or 0.1% DMSO for the PTX vehicle control.

At time points following drug administration, caspase-3/7 activation was measured as discussed in Example 1. The apoptosis inhibition index was calculated using data collected 66 hours after administration of PTX and/or DEX.

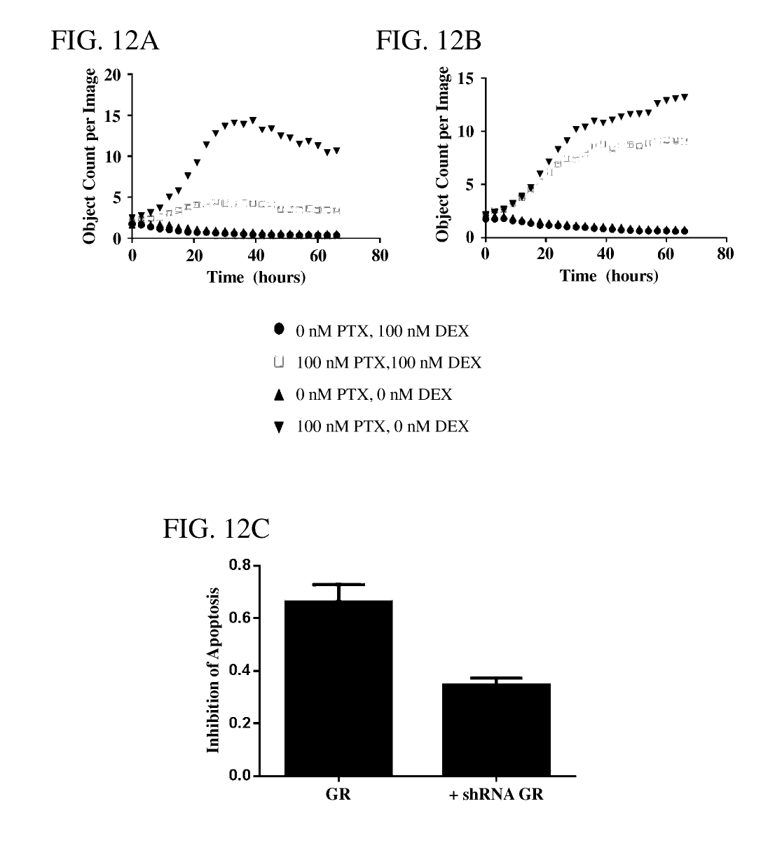

Administration of DEX inhibited PTX-induced apoptosis (FIG. 12A). Following GR knockdown, the level of DEX-mediated inhibition of PTX-induced apoptosis was reduced (FIG. 12B). As shown in FIG. 12C, following knockdown of GR, H1755 cells exhibited reduced DEX-mediated antagonism of PTX-induced apoptosis (as measured by the apoptosis inhibition index).

### Example 7

### Expression of NR3C1 in Solid Tumor Types

This example demonstrates the variation of NR3C1 expression (the gene for GR) across solid tumor types and within patients exhibiting a solid tumor type.

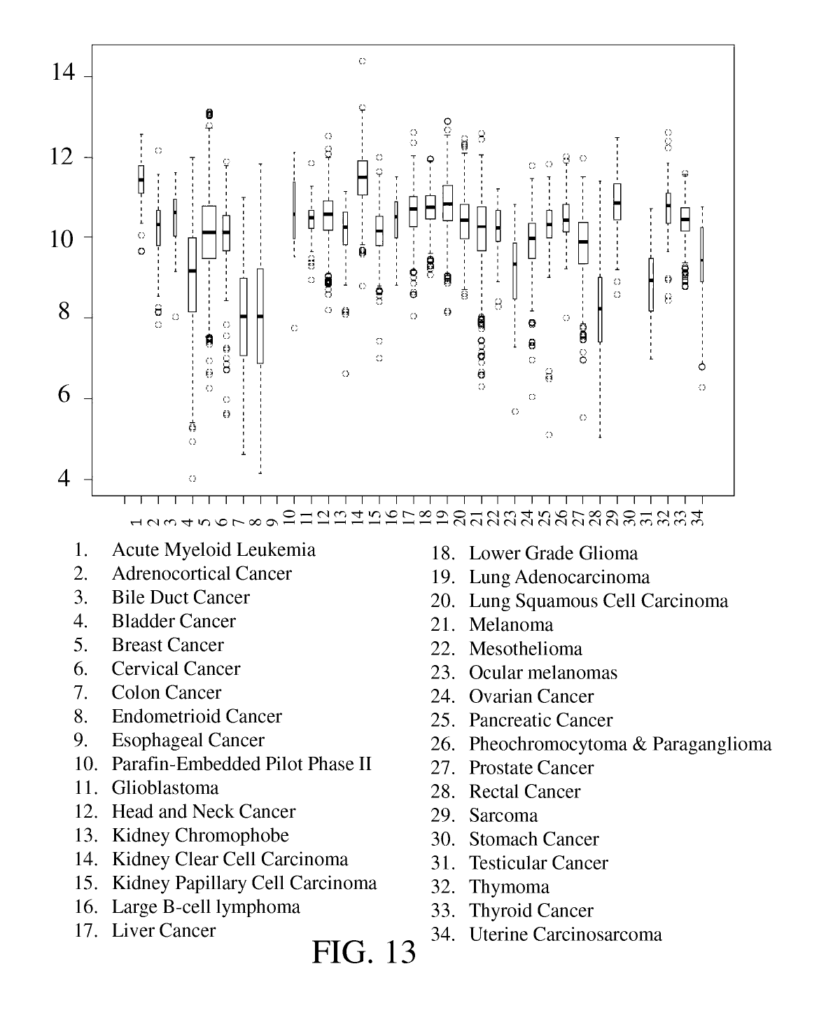

FIG. 13 illustrates the relative expression profile of NR3C1 in solid tumor samples from patients. The mRNA expression data of NR3C1 in cancer patients was obtained from The Cancer Genome Atlas (TCGA). TCGA computes the relative expression of an individual gene and tumor to the gene's expression distribution in a reference population. The reference population is either all tumors that are diploid for the gene in question (presumably within the same cohort), or, when available, normal adjacent tissue. Here, relative expression profiles of NR3C1 are derived from the PANCAN dataset provided by the TGCA.

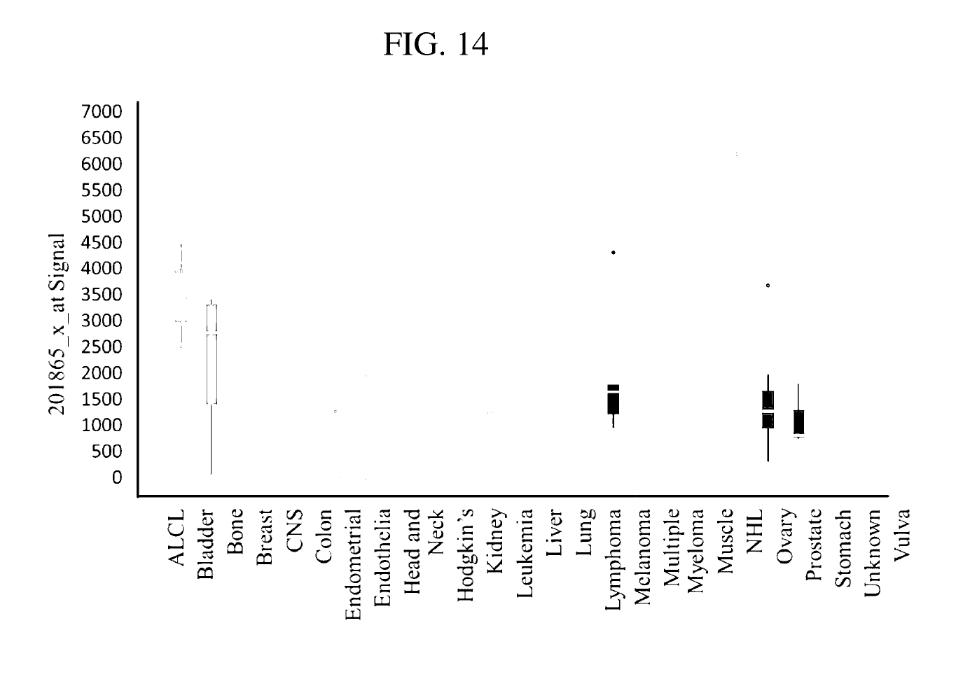

FIG. 14 illustrates the relative expression profile of NR3C1 for solid tumor cell lines. This data was sourced from the Cancer Cell Line Encyclopedia (a publically available dataset) and made available via a web-based querying tool.

### Example 8

### DEX Rescues PTX-Induced Apoptosis in H1755 Cells

This example demonstrates DEX rescue of PTX-induced apoptosis in H1755 cells.

H1755 cell samples were treated with 0, 0.43, 1.3, 4.12, 12.3, 37, 111, 333, 1000, or 3000 nM PTX. Each concentration level of PTX-treated H1755 cells were also treated with the following DEX concentrations: 0, 0.046, 0.137, 0.412, 1.235, 3.704, 11.111, 33.333, 100.000 nM. At time points following drug administration, caspase-3/7 activation was measured as discussed in Example 1.

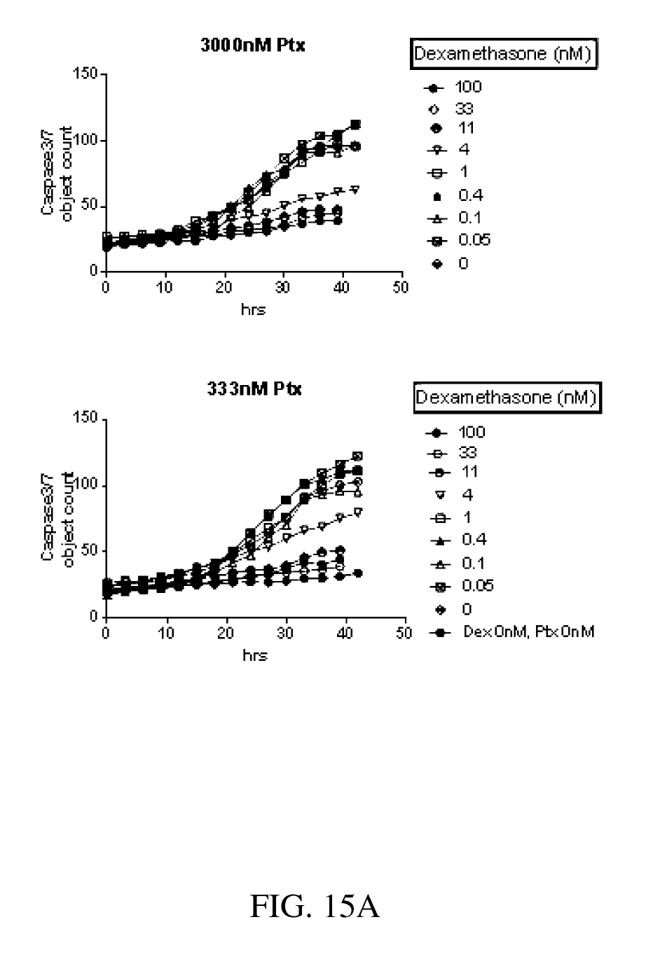

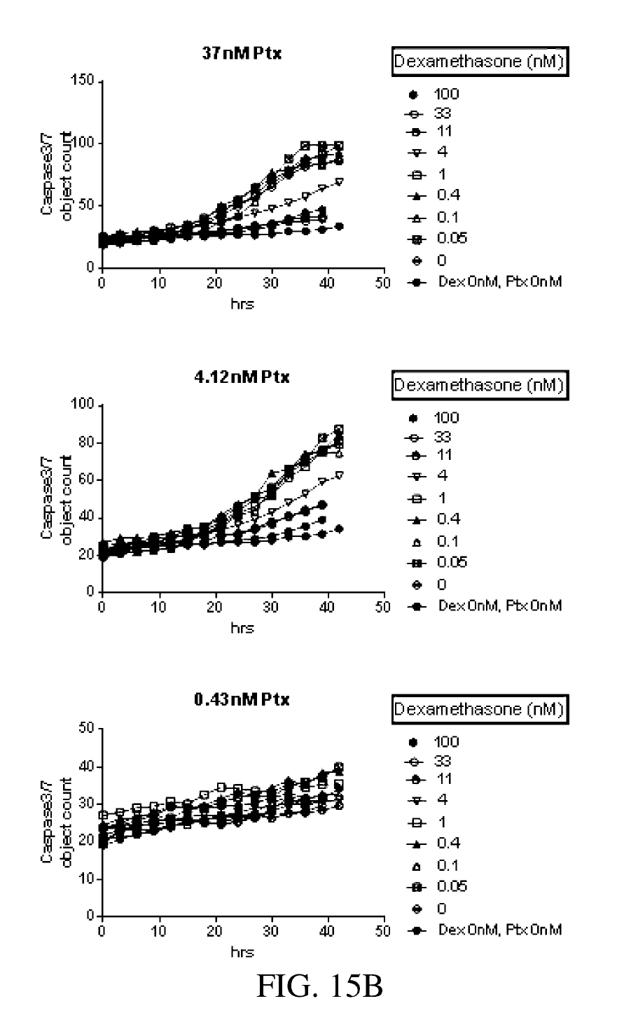

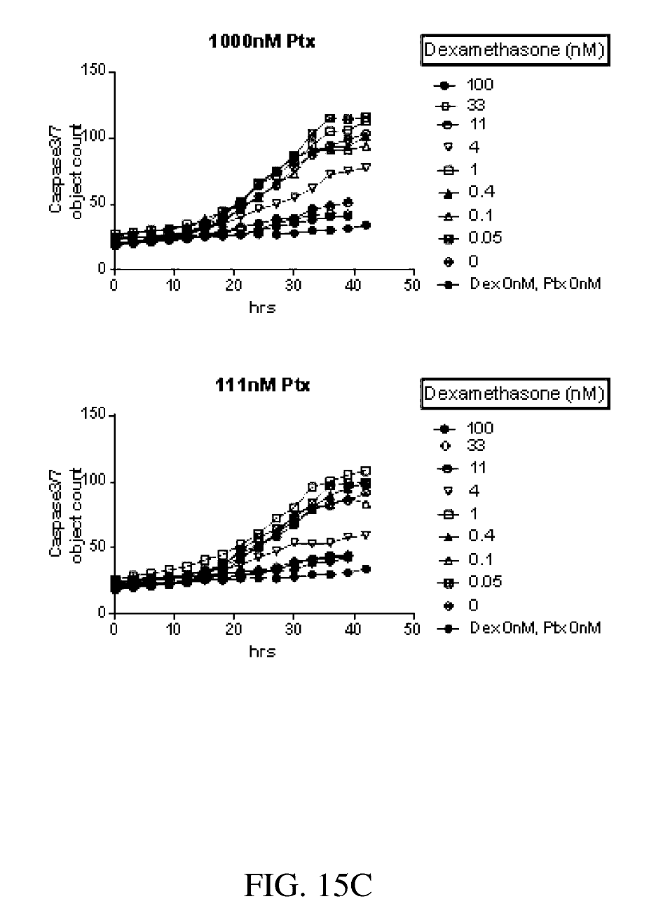

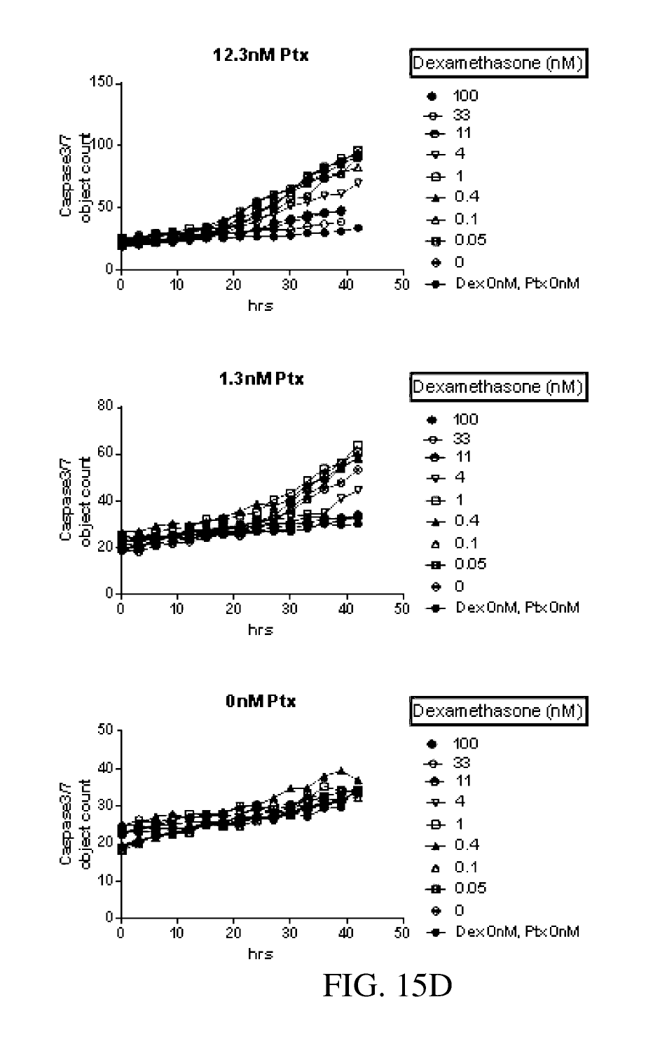

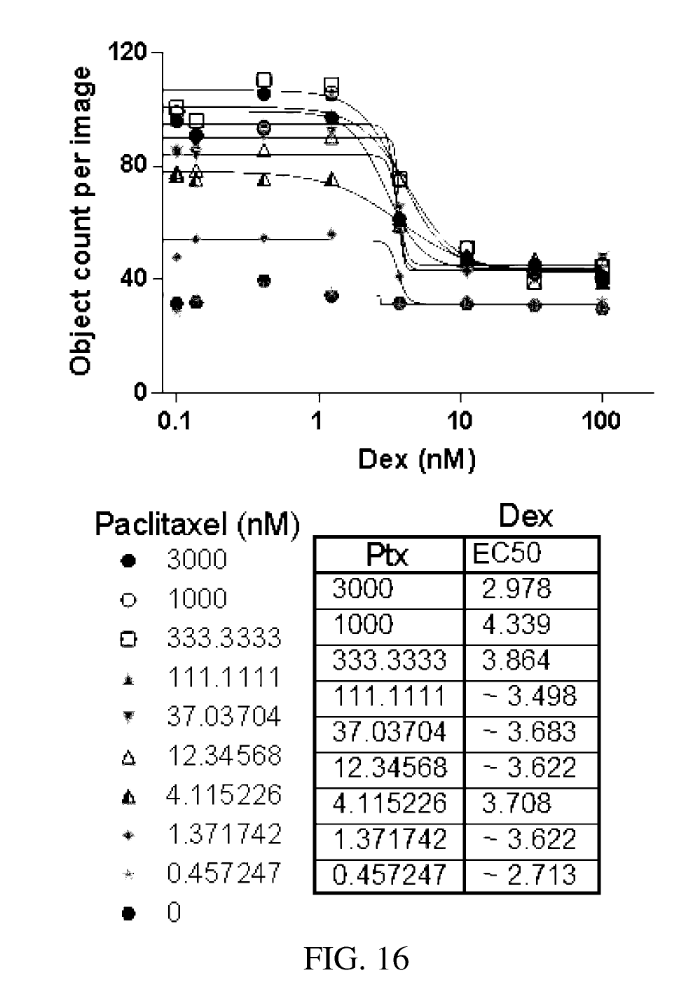

FIG. 15 shows a plot of DEX-mediated inhibition of PTX-induced apoptosis for each PTX concentration studied. FIG. 16 shows a plot of caspase-3/7 activation (i.e., the level of apoptosis) versus DEX concentration. The EC₅₀ for DEX rescue of H1755 was determined for each concentration of PTX treatment (FIG. 16). The EC₅₀ of anti-apoptotic effect of DEX in the H1755 cell line is below the measured blood levels and predicted tissue levels of DEX that occur following standard pre-medication for solvent-based paclitaxel, suggesting the anti-apoptotic effect observed in vitro may be clinically relevant.

### Example 9

### Effect of DEX and PTX on MAPK Stress Response Pathway

This example demonstrates that some MAPK pathway proteins and MAPK signaling mechanisms are minimally changed following DEX and/or PTX treatment.

H1755 and H522 cells were analyzed by Western blot at 4 hours following administration of a vehicle control (0.1% EtOH for DEX vehicle control or 0.1% DMSO for PTX vehicle control), 100 nM DEX, 100 nM PTX, or 100 nM DEX and 100 nM PTX.

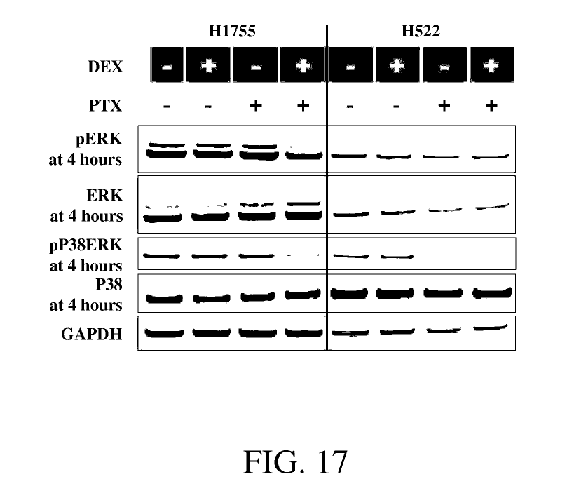

As shown in FIG. 17, H1755 cells, a GR positive cell line, and H522 cells, a GR negative cell line, exhibited minimal expression level changes for ERK, phosphorylated ERK, p38, and phosphorylated p38.

### Example 10

### Transcription Dependent and Independent Response to PTX and DEX

This example demonstrates protein expression level changes at 24 and 48 hours after administration of PTX and/or DEX for a series of proteins involved in stress response pathways to PTX and DEX.

H1755 and H522 cells were analyzed by Western blot at 4 hours following administration of a vehicle control (0.1% EtOH for DEX vehicle control or 0.1% DMSO for PTX vehicle control), 100 nM DEX, 100 nM PTX, or 100 nM DEX and 100 nM PTX.

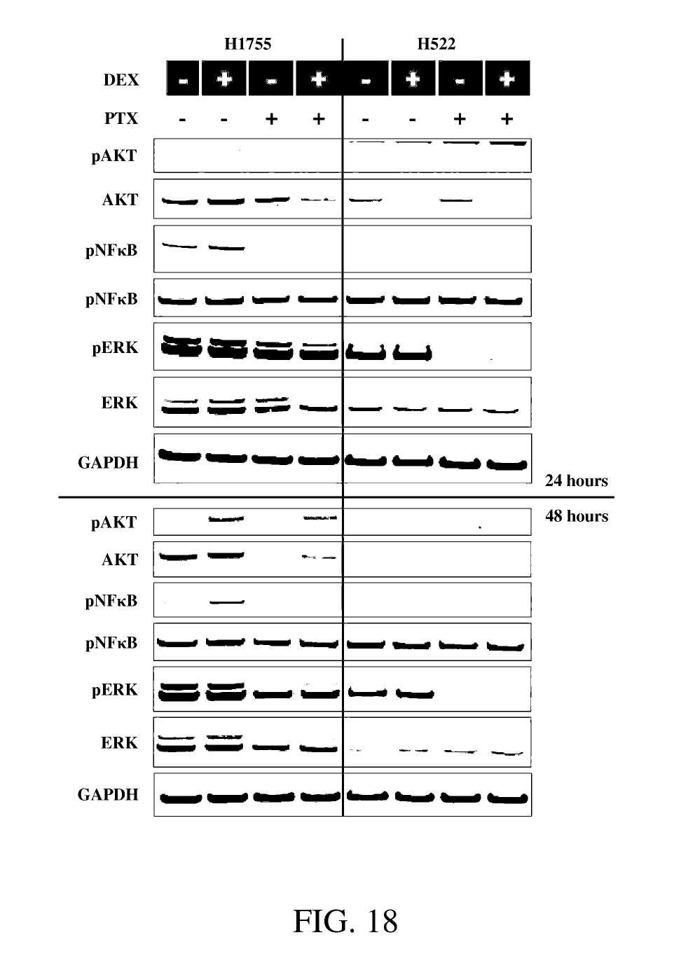

As shown in FIG. 18, H1755 cells, a GR positive cell line, and H522 cells, a GR negative cell line, exhibited variation in protein expression 48 hours after drug administration (as compared to 24 hours after drug administration).

### Example 11

### Efficacy of ABRAXANE® Alone or in Combination with Dexamethasone or Mifepristone in Treating Immunodeficient A549 Xenograft Mouse Model

This example aims to determine the efficacy of ABRAXANE® alone or in combination with dexamethasone or mifepristone (a GR antagonist) in treating A549 human NSCLC xenograft in female nude mice.

Female athymic nude mice (Crl:NU(NCr)-Foxn1^{Nu}, Charles River) between 8-12 weeks old are used on day 1 (D1) of the study. A549 tumor cells are cultured, harvested, and resuspended in cold PBS containing 50% MATRIGEL™ (BD Biosciences). Each mouse is injected subcutaneously in the right flank with 1×10⁷ cells (0.1 mL cell suspension) to implant the xenograft.

When the average volume of the xenograft tumor reaches the desired 150-200 mm³ range, a pair match among the mice is performed, and the mice are sorted into six groups to receive the treatment regimens on the next day (D1) as shown in Table 1. Vehicle, dexamethasone, and mifepristone are administered intraperitoneally (i.p.). ABRAXANE® is administered intravenously (i.v.). Group 1 serves as control, and receives vehicle daily for 10 days. Group 2 receives dexamethasone at 0.1 mg/kg daily for 10 days. Group 3 receives mifepristone at 15 mg/kg daily for 10 days. Group 4 receives vehicle daily for 10 days, and ABRAXANE® at 15 mg/kg every other day for five doses. Group 5 receives dexamethasone at 0.1 mg/kg daily for 10 days, and ABRAXANE® at 15 mg/kg every other day for five doses. Group 6 receives mifepristone at 15 mg/kg daily for 10 days, and ABRAXANE® at 1.5 mg/kg every other day for five doses. Dexamethasone and mifepristone are dosed 12 hours before ABRAXANE® on D1.

TABLE 1
Treatment groups of in vivo study in A549 xenograft mouse model.

| | | Regimen 1 | | | | | Regimen 2 | | | | |
|---|---|---|---|---|---|---|---|---|---|---|---|
| Gr. | N | Agent | Vehicle | mg/kg | Route | Schedule | Agent | Vehicle | mg/kg | Route | Schedule |
| 1 | 10 | vehicle | | — | ip | qd × 10 | — | — | — | — | — |
| 2 | 10 | dexamethasone | | 0.1 | ip | qd × 10 | — | — | — | — | — |
| 3 | 10 | mifepristone | | 15 | ip | qd × 10 | — | — | — | — | — |
| 4 | 10 | vehicle | | — | ip | qd × 10 | ABRAXANE ® | | 15 | iv | qod × 5 |
| 5 | 10 | dexamethasone | | 0.1 | ip | qd × 10 | ABRAXANE ® | | 15 | iv | qod × 5 |
| 6 | 10 | mifepristone | | 15 | ip | qd × 10 | ABRAXANE ® | | 15 | iv | qod × 5 |

ABRAXANE® is stored at −80° C. and protected from light during storage and formulation. Each dose of ABRAXANE® is freshly prepared and reconstituted in saline containing 1% HSA (human serum albumin). Dexamethasone is stored at 4° C., and protected from light. Dexamethasone dosing solution is prepared every week in saline. Mifepristone is prepared in 10% ethanol in sesame seed oil. Vehicle is 10% ethanol in sesame seed oil. Dosing volume is 10 mL/kg (0.200 mL/20 g mouse).

Animals are monitored individually. Tumors are measured with a caliper twice weekly for the duration of the study. Body weights of the animals are also measured five days a week for two weeks, and then biweekly for the duration of the study.

Any adverse reactions or deaths are reported immediately. Any individual animal with a single observation of greater than 30% body weight loss or three consecutive measurements of greater than 25% body weight loss are euthanized. Any group with a mean body weight loss of greater than 20% or greater than 10% mortality stops dosing. The group is not euthanized and recovery is allowed. Within a group with greater than 20% weight loss, individuals hitting the individual body weight loss endpoint are euthanized. If the group treatment related body weight loss is recovered to within 10% of the original weights, dosing may resume at a lower dose or less frequent dosing schedule. Exceptions to non-treatment body weight percentage recovery may be allowed on a case-by-case basis.

The endpoint of the experiment is a tumor volume of 1000 mm³ or 60 days, whichever comes first. Responders can be followed longer. When the endpoint is reached, the animals are euthanized. Data from various groups are compared, including individual Times to Endpoint (TTE), median or mean tumor volume over time, mean body weight changes over time, and survival (Kaplan-Meier analysis).

### Example 12

### Efficacy of ABRAXANE® Alone or in Combination with Dexamethasone or Mifepristone in Treating Immunodeficient H727 Xenograft Mouse Model

This example aims to determine the efficacy of ABRAXANE® alone or in combination with dexamethasone or mifepristone (a GR antagonist) in treating H727 human NSCLC xenograft in female nude mice.

Female athymic nude mice (Crl:NU(NCr)-Foxn1^{Nu}, Charles River) between 8-12 weeks old are used on day 1 (D1) of the study. H727 tumor cells are cultured, harvested, and resuspended in cold PBS containing 50% MATRIGEL™ (BD Biosciences). Each mouse is injected subcutaneously in the right flank with 5×10⁶ cells (0.1 mL cell suspension) to implant the xenograft.

When the average volume of the xenograft tumor reaches the desired 80-120 mm³ range, a pair match among the mice is performed, and the mice are sorted into six groups to receive the treatment regimens on the next day (D1) as shown in Table 1. Vehicle, dexamethasone, and mifepristone are administered intraperitoneally (i.p.). ABRAXANE® is administered intravenously (i.v.). Group 1 serves as control, and receives vehicle daily for 10 days. Group 2 receives dexamethasone at 0.1 mg/kg daily for 10 days. Group 3 receives mifepristone at 15 mg/kg daily for 10 days. Group 4 receives vehicle daily for 10 days, and ABRAXANE® at 30 mg/kg every other day for five doses. Group 5 receives dexamethasone at 0.1 mg/kg daily for 10 days, and ABRAXANE® at 30 mg/kg every other day for five doses. Group 6 receives mifepristone at 15 mg/kg daily for 10 days, and ABRAXANE® at 30 mg/kg every other day for five doses. Dexamethasone and mifepristone are dosed 12 hours before ABRAXANE® on D1.

TABLE 2
Treatment groups of in vivo study in H727 xenograft mouse model.

| | | Regimen 1 | | | | | Regimen 2 | | | | |
|---|---|---|---|---|---|---|---|---|---|---|---|
| Gr. | N | Agent | Vehicle | mg/kg | Route | Schedule | Agent | Vehicle | mg/kg | Route | Schedule |
| 1 | 10 | vehicle | | — | ip | qd × 10 | — | — | — | — | — |
| 2 | 10 | dexamethasone | | 0.1 | ip | qd × 10 | — | — | — | — | — |
| 3 | 10 | mifepristone | | 15 | ip | qd × 10 | — | — | — | — | — |
| 4 | 10 | vehicle | | — | ip | qd × 10 | ABRAXANE ® | | 30 | iv | qod × 5 (start on |
| | | | | | | | | | | | day 2) |
| 5 | 10 | dexamethasone | | 0.1 | ip | qd × 10 | ABRAXANE ® | | 15 | iv | qod × 5 (start on |
| | | | | | | | | | | | day 2) |
| 6 | 10 | mifepristone | | 15 | ip | qd × 10 | ABRAXANE ® | | 15 | iv | qod × 5 (start on |
| | | | | | | | | | | | day 2) |

ABRAXANE® is stored at −80° C. and protected from light during storage and formulation. Each dose of ABRAXANE® is freshly prepared and reconstituted in saline containing 1% HSA (human serum albumin). Dexamethasone is stored at 4° C., and protected from light. Dexamethasone dosing solution is prepared every week in saline. Mifepristone is prepared in 10% ethanol in sesame seed oil. Vehicle is 10% ethanol in sesame seed oil. Dosing volume is 10 mL/kg (0.200 mL/20 g mouse).

Animals are monitored individually. Tumors are measured with a caliper twice weekly for the duration of the study. Body weights of the animals are also measured five days a week for two weeks, and then biweekly for the duration of the study.

Any adverse reactions or deaths are reported immediately. Any individual animal with a single observation of greater than 30% body weight loss or three consecutive measurements of greater than 25% body weight loss are euthanized. Any group with a mean body weight loss of greater than 20% or greater than 10% mortality stops dosing. The group is not euthanized and recovery is allowed. Within a group with greater than 20% weight loss, individuals hitting the individual body weight loss endpoint are euthanized. If the group treatment related body weight loss is recovered to within 10% of the original weights, dosing may resume at a lower dose or less frequent dosing schedule. Exceptions to non-treatment body weight percentage recovery may be allowed on a case-by-case basis.

The endpoint of the experiment is a tumor volume of 2000 mm³ or 60 days, whichever comes first. Responders can be followed longer. When the endpoint is reached, the animals are euthanized. Data from various groups are compared, including individual Times to Endpoint (TTE), median or mean tumor volume over time, mean body weight changes over time, and survival (Kaplan-Meier analysis).

### Example 13

### Effect of DEX on Gene Expression in 8 Cancer Cell Lines

This example demonstrates that genes functioning in epithelial-mesenchymal transition (EMT), apoptosis and inflammation responses are consistently modulated by dexamethasone treatment in various GR-positive cancer cell lines.

H1755, a GR positive cancer cell line, was treated by 100 nM DEX for 24 hours. Expression levels of 357 genes from 5 pathways (EMT, apoptosis, stem cell, cancer inflammation and immunity cross talk, and IL6/STAT3 signaling pathway) in DEX-treated H1755 cells and untreated H1755 cells (control) were determined using RT² Profiler PCR Arrays (Qiagen). 46 genes were found to show at least two fold change in response to the DEX treatment.

The data from the H1755 cell line as well as results described in Examples 2-6, and 8-10 suggest that the effect of DEX in the H1755 cancer cell line may be mediated by inhibiting apoptosis pathway (e.g., via caspase activation, BCL2), inhibiting stress response (e.g., via JNK, cJun, MKP1, NFκb), and activating EMT like mechanisms (e.g., via SLUG, FoxC2, vimentin).

The expression levels of the 46 genes were determined in 7 additional cancer cell lines under DEX treatment (100 nM for 24 hours) or no DEX (control) conditions. The 7 cancer cell lines included CAL120, A549, BT549, H727, Hs766T, AsPC1, and LLC1 (mouse Lewis lung carcinoma).

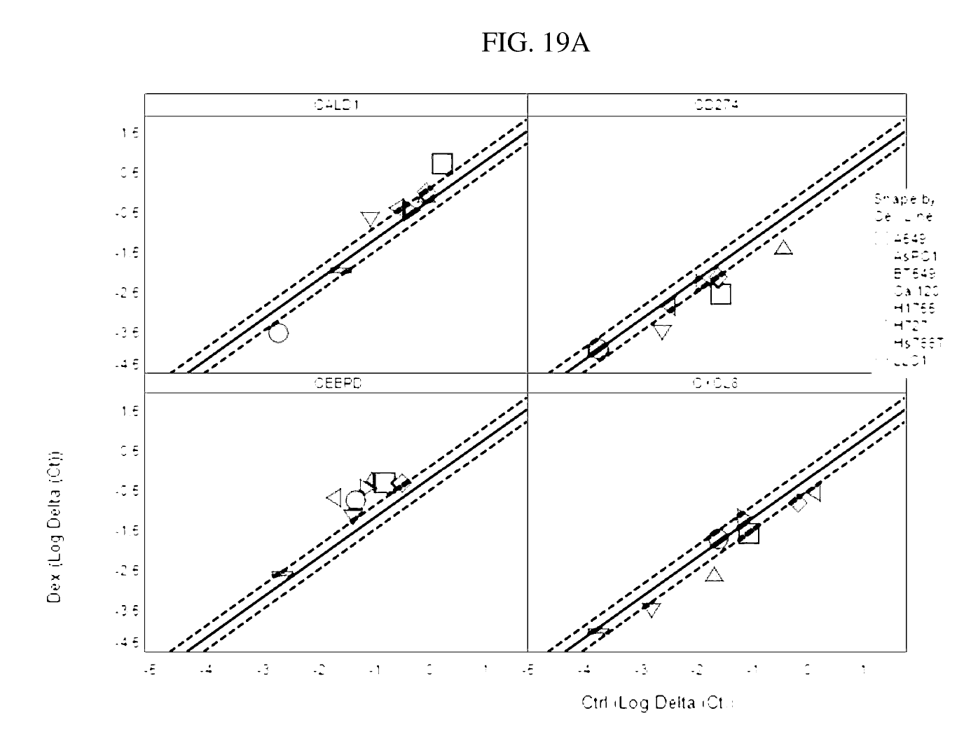

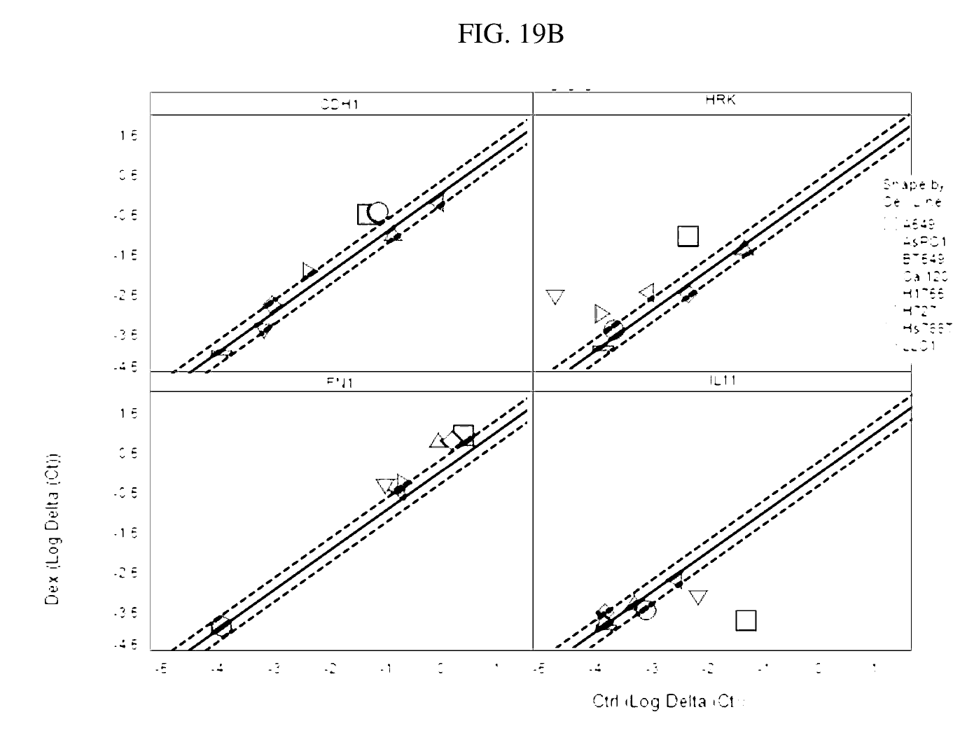

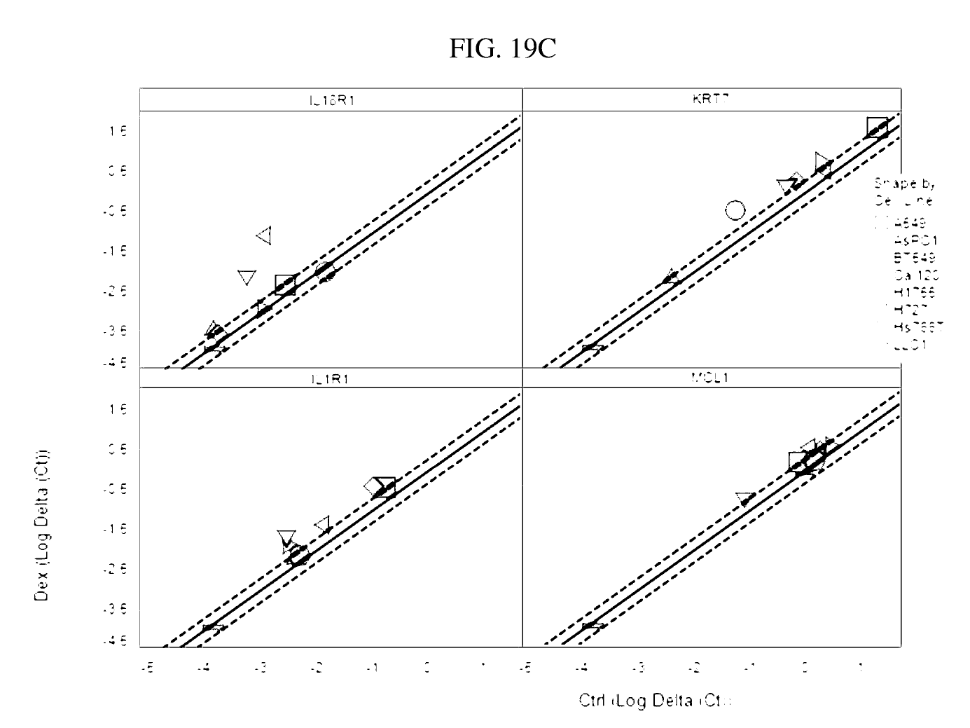

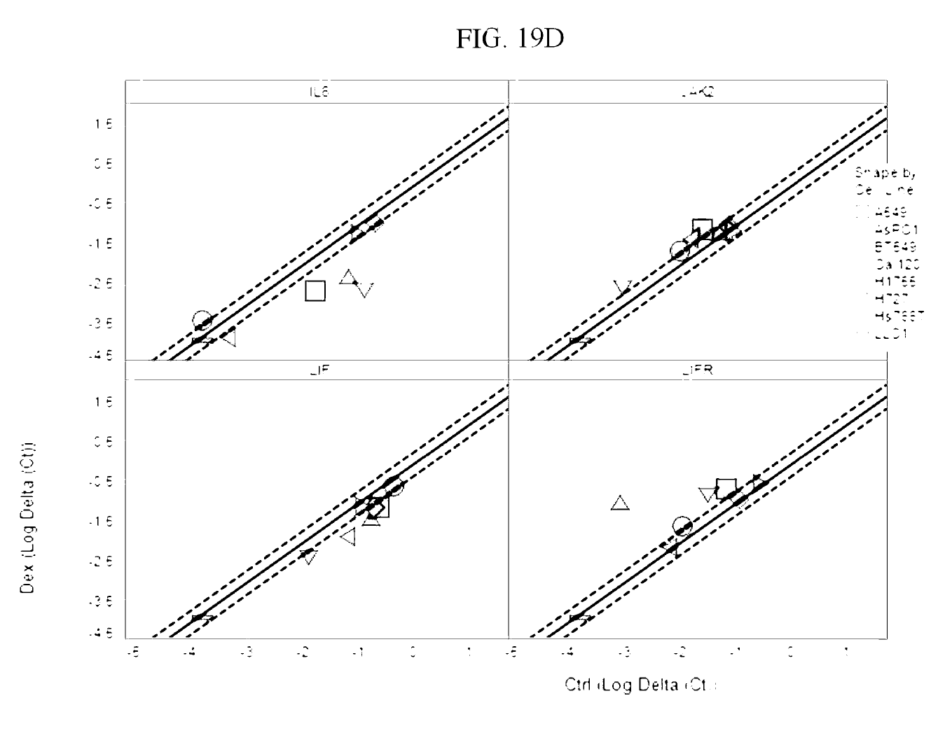

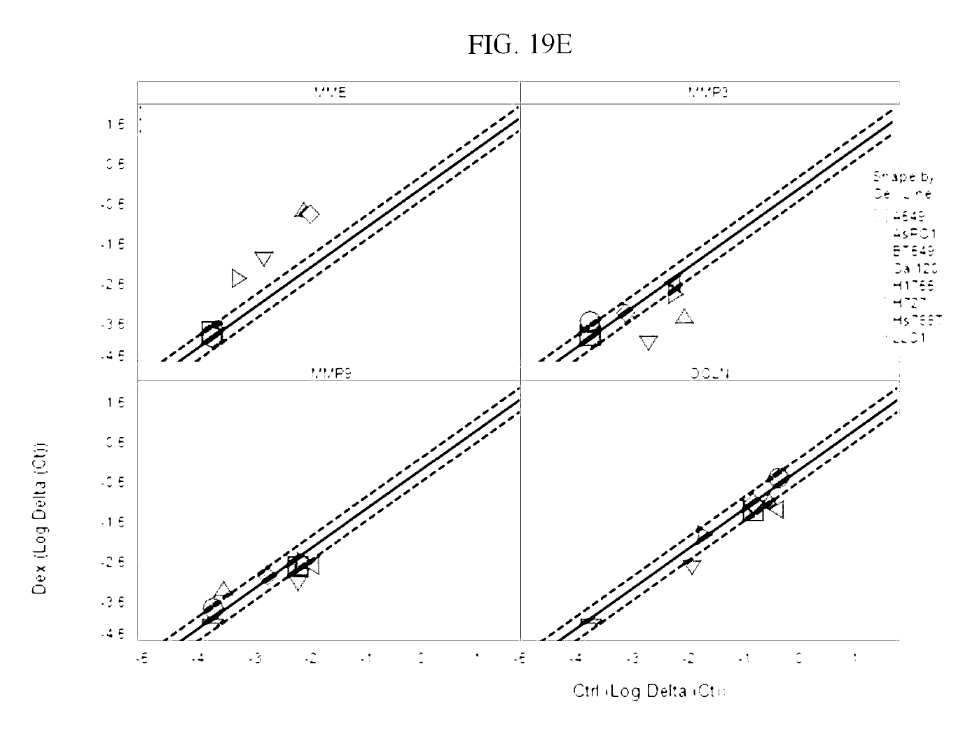

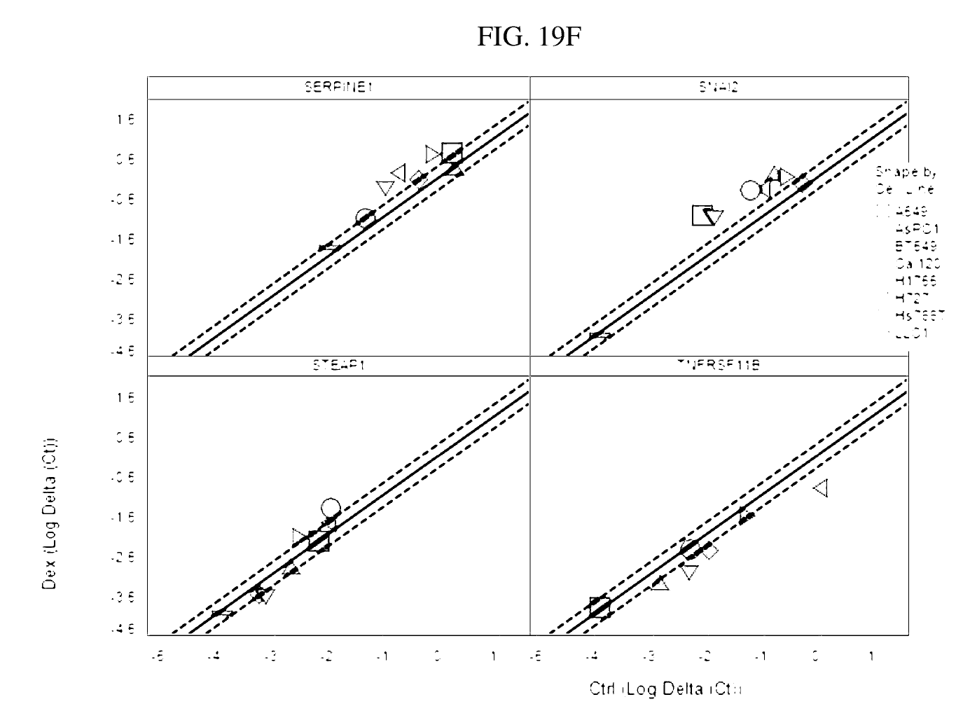

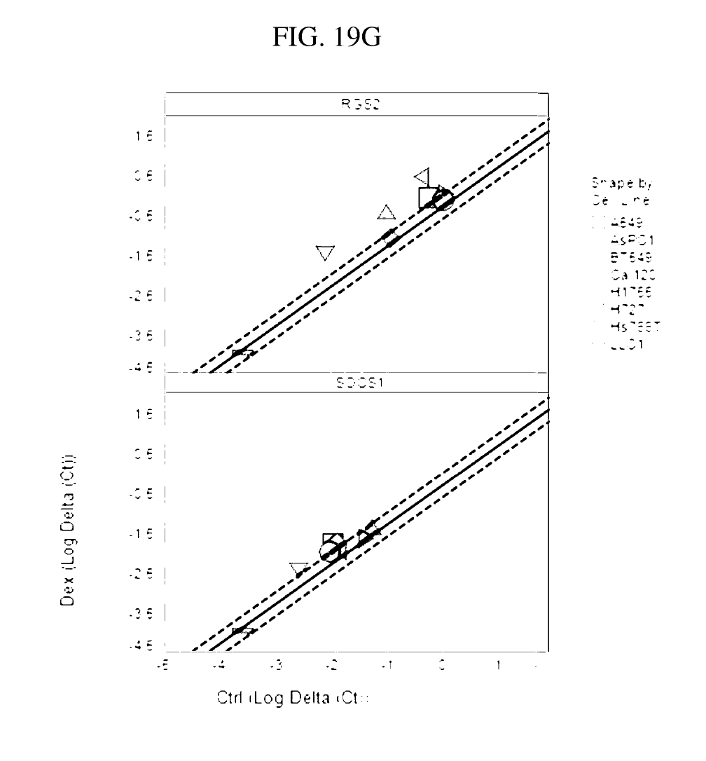

FIGS. 19A-19C show the expression levels of 26 genes in each of the 7 cell lines plus H1755, with the x-axis showing the ΔCt in untreated cells, and the y-axis showing the ΔCt in DEX-treated cells. In each panel, a data paint in the top left region above the dotted line indicates upregulation of the gene by at least two fold in response to the DEX treatment, while a data point in the bottom left region below the dotted line indicates downregulation by at least two fold in response to the DEX treatment. Each of the 26 genes was upregulated or downregulated by at least two fold in response to the DEX treatment in at least one of the 7 cell lines.

13 of the 26 genes are known to function in EMT, apoptosis, and inflammation response. EMT genes (FN1, SERPINE1, SNA1 (also known as SLUG) and RGS2), inflammation response genes (IL1R1, JAK2, CEBPD), KRT7 (a stem cell marker), MME (a differentiation marker), and MCL (an anti-apoptotic gene) are consistently upregulated by DEX in the 7 tested GR-positive human cell lines. Inflammation response and apoptosis genes IL6, LIF, and TNFRSF11B are consistently downregulated by DEX in the 7 tested GR-positive human cell lines. Importantly, previous studies have implicated regulatory roles of SLUG in EMT, tumor growth and metastasis. For example, Guo W et al. Cell (2012) 148(5): 1015-1028 demonstrated that over-expression of SLUG induces EMT, and knock-down of SLUG inhibits mammary cell spheroid formation, tumor growth and metastasis.

The expression data suggests that the effect of DEX on the cancer cell lines may be mediated through inhibiting inflammation and apoptosis pathways, and activating EMT-like mechanisms (for example, via SLUG).

1. A method of treating an individual having a cancer, wherein the individual is characterized by a high level of glucocorticoid receptor (GR), comprising administering to the individual an effective amount of a composition comprising a taxane.

2. A method of treating an individual having a cancer, wherein the individual is characterized by a high level of glucocorticoid (GC), comprising administering to the individual an effective amount of a composition comprising a taxane.

3. (canceled)

4. A method of treating an individual having a cancer, comprising administering to the individual: a) an effective amount of a composition comprising a taxane; and b) an effective amount of another agent that down-regulates glucocorticoid receptor (GR).

5-10. (canceled)

11. The method of claim 4, wherein the individual is characterized by a high level of GR expression.

12. The method of claim 4, wherein the individual is characterized by a high level of GR activity.

13. The method of claim 12, wherein the high level of GR activity is determined by measuring the expression or activity of a GR responsive molecule.

14. The method of claim 4, wherein the individual is characterized by a high level of GC secretion.

15. The method of claim 4, wherein the individual is characterized by high level of GC activity.

16. The method of claim 4, wherein the other agent is an inhibitor of GR expression.

17. The method of claim 4, wherein the other agent is an inhibitor of GR activity.

18. The method of claim 17, wherein the other agent is a GR antagonist.

19. The method of claim 17, wherein the other agent is a modulator of a GR responsive molecule.

20. (canceled)

21. The method of claim 4, wherein the cancer is selected from the group consisting of breast cancer, lung cancer, and pancreatic cancer.

22. The method of claim 21, wherein the cancer is pancreatic cancer.

23. The method claim 4, wherein the cancer is advanced cancer.

24.-26. (canceled)

27. The method of claim 4, wherein the taxane is paclitaxel.

28. The method of claim 4, wherein the composition comprises nanoparticles comprising the taxane.

29. The method of claim 28, wherein the composition comprises nanoparticles comprising the taxane and an albumin.

30. (canceled)

31. The method of claim 28, wherein the nanoparticles in the composition have an average diameter of no greater than about 200 nm.

32. (canceled)

33. The method of claim 4, wherein the individual is human.
